SEQUENCE LISTING (160 sequences)

SEQ ID NO: 1 - DNA, 3 bp, Artificial Sequence
  Synthetic
  misc_feature (1)..(1): n is independently a or c or g or t
ngg

SEQ ID NO: 2 - DNA, 3 bp, Artificial Sequence
  Synthetic
  misc_feature (1)..(1): n is independently a or c or g or t
nga

SEQ ID NO: 3 - DNA, 4 bp, Artificial Sequence
  Synthetic
  misc_feature (1)..(1): n is independently a or c or g or t
  misc_feature (4)..(4): n is independently a or c or g or t
ngan

SEQ ID NO: 4 - DNA, 4 bp, Artificial Sequence
  Synthetic
  misc_feature (1)..(1): n is independently a or c or g or t
  misc_feature (3)..(3): n is independently a or c or g or t
ngng

SEQ ID NO: 5 - DNA, 5 bp, Artificial Sequence
  Synthetic
  misc_feature (1)..(1): n is independently a or c or g or t
  misc_feature (4)..(4): n is independently a or c or g or t
nggng

SEQ ID NO: 6 - DNA, 7 bp, Artificial Sequence
  Synthetic
  misc_feature (1)..(2): n is independently a or c or g or t
  misc_feature (7)..(7): w is a or t
nnagaaw

SEQ ID NO: 7 - DNA, 4 bp, Artificial Sequence
  Synthetic
  misc_feature (1)..(1): n is independently a or c or g or t
naar

SEQ ID NO: 8 - DNA, 5 bp, Artificial Sequence
  Synthetic
  misc_feature (1)..(2): n is independently a or c or g or t
nngrr

SEQ ID NO: 9 - DNA, 6 bp, Artificial Sequence
  Synthetic
  misc_feature (1)..(2): n is independently a or c or g or t
  misc_feature (6)..(6): n is independently a or c or g or t
nngrrn

SEQ ID NO: 10 - DNA, 6 bp, Artificial Sequence
  Synthetic
  misc_feature (1)..(2): n is independently a or c or g or t
nngrrt

SEQ ID NO: 11 - DNA, 6 bp, Artificial Sequence
  Synthetic
  misc_feature (1)..(2): n is independently a or c or g or t
nngrrv

SEQ ID NO: 12 - DNA, 8 bp, Artificial Sequence
  Synthetic
  misc_feature (1)..(4): n is independently a or c or g or t
nnnngatt

SEQ ID NO: 13 - DNA, 8 bp, Artificial Sequence
  Synthetic
  misc_feature (1)..(4): n is independently a or c or g or t
  misc_feature (6)..(8): n is independently a or c or g or t
nnnngnnn

SEQ ID NO: 14 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 15 - protein, 1368 aa, Artificial Sequence [sequence omitted]

SEQ ID NO: 16 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 17 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 18 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 19 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 20 - DNA, 3239 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaa

SEQ ID NO: 21 - protein, 1053 aa, Artificial Sequence
  Synthetic
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 23 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 24 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 25 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 26 - protein, 1052 aa, Artificial Sequence
  Synthetic
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 27 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 28 - protein, 252 aa, Artificial Sequence
  Synthetic
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKPKKKRKVGGPKKKRKV

SEQ ID NO: 29 - DNA, 759 bp, Artificial Sequence
  Synthetic
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagcccaagaagaagaggaaggtgggtggccctaagaaaaagagaaaggtgtga

SEQ ID NO: 30 - DNA, 52 bp, Artificial Sequence
  Synthetic
aatgatacggcgaccaccgagatctacacaatttcttgggtagtttgcagtt

SEQ ID NO: 31 - DNA, 51 bp, Artificial Sequence
  Synthetic
  misc_feature (25)..(30): n is independently a or c or g or t
caagcagaagacggcatacgagatnnnnnngactcggtgccactttttcaa

SEQ ID NO: 32 - DNA, 43 bp, Artificial Sequence
  Synthetic
gatttcttggctttatatatcttgtggaaaggacgaaacaccg

SEQ ID NO: 33 - DNA, 38 bp, Artificial Sequence
  Synthetic
gctagtccgttatcaacttgaaaaagtggcaccgagtc

SEQ ID NO: 34 - DNA, 60 bp, Artificial Sequence
  Synthetic
gttgataacggactagccttatttaaacttgctatgctgtttccagcatagctcttaaac

SEQ ID NO: 35 - DNA, 4 bp, Artificial Sequence
  Synthetic
  misc_feature (4)..(4): n is independently a or c or g or t
tttn

SEQ ID NO: 36 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 37 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 38 - DNA, 19 bp, Artificial Sequence
  Synthetic
tgtcgtgatgcgtagacgg

SEQ ID NO: 39 - DNA, 19 bp, Artificial Sequence
  Synthetic
tcatcaaggagcattccgt

SEQ ID NO: 40 - DNA, 19 bp, Artificial Sequence
  Synthetic
ctcgagagagcaaacagag

SEQ ID NO: 41 - DNA, 19 bp, Artificial Sequence
  Synthetic
atagaagggggaagtcgga

SEQ ID NO: 42 - DNA, 19 bp, Artificial Sequence
  Synthetic
catgccaaacccctccccc

SEQ ID NO: 43 - DNA, 19 bp, Artificial Sequence
  Synthetic
tgaggggagcggttgtcgg

SEQ ID NO: 44 - DNA, 19 bp, Artificial Sequence
  Synthetic
cgccgggcggggcgaccag

SEQ ID NO: 45 - DNA, 19 bp, Artificial Sequence
  Synthetic
agcgcgagcgcaagggaca

SEQ ID NO: 46 - DNA, 19 bp, Artificial Sequence
  Synthetic
ctgggtgacgaggcgggag

SEQ ID NO: 47 - DNA, 19 bp, Artificial Sequence
  Synthetic
caaggctacacctgccccc

SEQ ID NO: 48 - DNA, 19 bp, Artificial Sequence
  Synthetic
tagcccgagccgactcccg

SEQ ID NO: 49 - DNA, 19 bp, Artificial Sequence
  Synthetic
gcgcgcgccgtgaggtcat

SEQ ID NO: 50 - DNA, 19 bp, Artificial Sequence
  Synthetic
ctcccttccatcgttgcta

SEQ ID NO: 51 - DNA, 19 bp, Artificial Sequence
  Synthetic
ccgggccgggaatttggag

SEQ ID NO: 52 - DNA, 19 bp, Artificial Sequence
  Synthetic
cgacaggtaacaaataggt

SEQ ID NO: 53 - DNA, 19 bp, Artificial Sequence
  Synthetic
aatacccctatctatctgg

SEQ ID NO: 54 - DNA, 19 bp, Artificial Sequence
  Synthetic
gcctgcccgcgccctccat

SEQ ID NO: 55 - DNA, 19 bp, Artificial Sequence
  Synthetic
gcagcgaggacgaaggcgg

SEQ ID NO: 56 - DNA, 19 bp, Artificial Sequence
  Synthetic
ggaaaggcggtgaagaaag

SEQ ID NO: 57 - DNA, 19 bp, Artificial Sequence
  Synthetic
gcctcagcggaatcccgcc

SEQ ID NO: 58 - DNA, 19 bp, Artificial Sequence
  Synthetic
gaggaggagggggagttta

SEQ ID NO: 59 - DNA, 20 bp, Artificial Sequence
  Synthetic
aatggagagtttgcaaggag

SEQ ID NO: 60 - DNA, 19 bp, Artificial Sequence
  Synthetic
actaacacaccatctggag

SEQ ID NO: 61 - DNA, 19 bp, Artificial Sequence
  Synthetic
cggggcggttgtgcaggag

SEQ ID NO: 62 - DNA, 19 bp, Artificial Sequence
  Synthetic
ggctgagaagacacgcgac

SEQ ID NO: 63 - DNA, 19 bp, Artificial Sequence
  Synthetic
cactcggagatcacacacc

SEQ ID NO: 64 - DNA, 19 bp, Artificial Sequence
  Synthetic
cacgcgaccggcgcgagga

SEQ ID NO: 65 - DNA, 19 bp, Artificial Sequence
  Synthetic
tgcggagccggctctcggc

SEQ ID NO: 66 - DNA, 19 bp, Artificial Sequence
  Synthetic
agagaaacaccaacaaaga

SEQ ID NO: 67 - DNA, 19 bp, Artificial Sequence
  Synthetic
ggcctgcagagtcacgtgg

SEQ ID NO: 68 - DNA, 19 bp, Artificial Sequence
  Synthetic
tgcccccacgtgactctgc

SEQ ID NO: 69 - DNA, 19 bp, Artificial Sequence
  Synthetic
gggccaccggaggcccaat

SEQ ID NO: 70 - DNA, 19 bp, Artificial Sequence
  Synthetic
aggaggaggactaccaaga

SEQ ID NO: 71 - DNA, 19 bp, Artificial Sequence
  Synthetic
cggggaacaccgggctaaa

SEQ ID NO: 72 - DNA, 19 bp, Artificial Sequence
  Synthetic
gcgccaagactccgagggg

SEQ ID NO: 73 - DNA, 19 bp, Artificial Sequence
  Synthetic
cctgccggaggccgcccaa

SEQ ID NO: 74 - DNA, 19 bp, Artificial Sequence
  Synthetic
ccacccaaaggcaactcag

SEQ ID NO: 75 - DNA, 19 bp, Artificial Sequence
  Synthetic
ggatacaaaggtttctcag

SEQ ID NO: 76 - DNA, 19 bp, Artificial Sequence
  Synthetic
aggccctcggcgcgctctg

SEQ ID NO: 77 - DNA, 19 bp, Artificial Sequence
  Synthetic
ccctctagagcaagatgag

SEQ ID NO: 78 - DNA, 19 bp, Artificial Sequence
  Synthetic
ggcgtccttaaacctcagg

SEQ ID NO: 79 - DNA, 19 bp, Artificial Sequence
  Synthetic
acagtcccaggaacggagg

SEQ ID NO: 80 - DNA, 19 bp, Artificial Sequence
  Synthetic
gtggaccgcgcccccccat

SEQ ID NO: 81 - DNA, 19 bp, Artificial Sequence
  Synthetic
ccctctaggacccggcacg

SEQ ID NO: 82 - DNA, 19 bp, Artificial Sequence
  Synthetic
agaagaggggccccggaga

SEQ ID NO: 83 - DNA, 19 bp, Artificial Sequence
  Synthetic
ggaccctgcagcaaagccc

SEQ ID NO: 84 - DNA, 19 bp, Artificial Sequence
  Synthetic
ccgcccgctcggggatccc

SEQ ID NO: 85 - DNA, 19 bp, Artificial Sequence
  Synthetic
cttccctacccggcgcttc

SEQ ID NO: 86 - DNA, 20 bp, Artificial Sequence
  Synthetic
gagcgtgtgtgtgagtgcgc

SEQ ID NO: 87 - DNA, 20 bp, Artificial Sequence
  Synthetic
cggctgtgctagcaatctgg

SEQ ID NO: 88 - DNA, 19 bp, Artificial Sequence
  Synthetic
gagccctcctatctatcct

SEQ ID NO: 89 - DNA, 20 bp, Artificial Sequence
  Synthetic
gctgtgcgccgtgcccgccc

SEQ ID NO: 90 - DNA, 19 bp, Artificial Sequence
  Synthetic
cggaggacccgtgattgac

SEQ ID NO: 91 - DNA, 20 bp, Artificial Sequence
  Synthetic
gctcggctcattcccgcccg

SEQ ID NO: 92 - DNA, 19 bp, Artificial Sequence
  Synthetic
ccctgagtgttggggatga

SEQ ID NO: 93 - DNA, 20 bp, Artificial Sequence
  Synthetic
tcccgtggctcccggcccgg

SEQ ID NO: 94 - DNA, 20 bp, Artificial Sequence
  Synthetic
gccccggccgctctagcccg

SEQ ID NO: 95 - DNA, 19 bp, Artificial Sequence
  Synthetic
tcatcaaggagcattccgt

SEQ ID NO: 96 - DNA, 20 bp, Artificial Sequence
  Synthetic
atgacaacaagaaccccgga

SEQ ID NO: 97 - DNA, 20 bp, Artificial Sequence
  Synthetic
cccttccccgggaggtgtgg

SEQ ID NO: 98 - DNA, 19 bp, Artificial Sequence
  Synthetic
cggctaggaggcgggtgga

SEQ ID NO: 99 - DNA, 19 bp, Artificial Sequence
  Synthetic
cgggccaaccttctctcct

SEQ ID NO: 100 - DNA, 19 bp, Artificial Sequence
  Synthetic
cgcgcacgccagtgtggag

SEQ ID NO: 101 - DNA, 19 bp, Artificial Sequence
  Synthetic
gggccatgcgggagaaaga

SEQ ID NO: 102 - DNA, 19 bp, Artificial Sequence
  Synthetic
ggaggggatcgcagccaaa

SEQ ID NO: 103 - DNA, 19 bp, Artificial Sequence
  Synthetic
ggaggagccctgagtgttg

SEQ ID NO: 104 - DNA, 19 bp, Artificial Sequence
  Synthetic
gcaagcagctggagagcgg

SEQ ID NO: 105 - DNA, 20 bp, Artificial Sequence
  Synthetic
aacccagtgcacctaagctc

SEQ ID NO: 106 - DNA, 20 bp, Artificial Sequence
  Synthetic
ggacttcagcatgacgtggt

SEQ ID NO: 107 - DNA, 21 bp, Artificial Sequence
  Synthetic
cagcttgtctctaaccgagga

SEQ ID NO: 108 - DNA, 21 bp, Artificial Sequence
  Synthetic
tgtgtcgtgttctcaaagggt

SEQ ID NO: 109 - DNA, 20 bp, Artificial Sequence
  Synthetic
tttggacatctcttcaggcc

SEQ ID NO: 110 - DNA, 19 bp, Artificial Sequence
  Synthetic
tttcacactccttccgcac

SEQ ID NO: 111 - DNA, 20 bp, Artificial Sequence
  Synthetic
ccgagtcccctcacatgttt

SEQ ID NO: 112 - DNA, 20 bp, Artificial Sequence
  Synthetic
ttgagttgtccaaggtcggg

SEQ ID NO: 113 - DNA, 20 bp, Artificial Sequence
  Synthetic
gaaagtctccctggctcgtc

SEQ ID NO: 114 - DNA, 20 bp, Artificial Sequence
  Synthetic
ccaaatagggagcgccttca

SEQ ID NO: 115 - DNA, 20 bp, Artificial Sequence
  Synthetic
ttttctcctttggggctggg

SEQ ID NO: 116 - DNA, 20 bp, Artificial Sequence
  Synthetic
aggcgtcatcctttctaccg

SEQ ID NO: 117 - DNA, 20 bp, Artificial Sequence
  Synthetic
gacgagcggccattcatcag

SEQ ID NO: 118 - DNA, 20 bp, Artificial Sequence
  Synthetic
cactcagtaatccagccggg

SEQ ID NO: 119 - DNA, 20 bp, Artificial Sequence
  Synthetic
aacagggcggctggttaata

SEQ ID NO: 120 - DNA, 20 bp, Artificial Sequence
  Synthetic
acactggtggcaggttaagg

SEQ ID NO: 121 - DNA, 20 bp, Artificial Sequence
  Synthetic
gatgactaaggctcgcctgg

SEQ ID NO: 122 - DNA, 20 bp, Artificial Sequence
  Synthetic
agaatagcaaggcaccacct

SEQ ID NO: 123 - DNA, 20 bp, Artificial Sequence
  Synthetic
tacgcgtttgtctcgtggtt

SEQ ID NO: 124 - DNA, 20 bp, Artificial Sequence
  Synthetic
cagagattggtaggcgaggc

SEQ ID NO: 125 - DNA, 20 bp, Artificial Sequence
  Synthetic
ttgcattaggagcgaacagc

SEQ ID NO: 126 - DNA, 20 bp, Artificial Sequence
  Synthetic
aaaaggggacttctccacgg

SEQ ID NO: 127 - DNA, 20 bp, Artificial Sequence
  Synthetic
taaaacggtgctgctgggaa

SEQ ID NO: 128 - DNA, 20 bp, Artificial Sequence
  Synthetic
agtgtgctcgggcacttatt

SEQ ID NO: 129 - DNA, 20 bp, Artificial Sequence
  Synthetic
gacatgaaggtgaagggcga

SEQ ID NO: 130 - DNA, 20 bp, Artificial Sequence
  Synthetic
cgttgtgcaggtctggattc

SEQ ID NO: 131 - DNA, 20 bp, Artificial Sequence
  Synthetic
aatatctcccgggcgtctga

SEQ ID NO: 132 - DNA, 20 bp, Artificial Sequence
  Synthetic
gttcaagttgtgcatgcggt

SEQ ID NO: 133 - DNA, 21 bp, Artificial Sequence
  Synthetic
cttctaggggaagaaccgagg

SEQ ID NO: 134 - DNA, 20 bp, Artificial Sequence
  Synthetic
aagaggacgaggagcgtttc

SEQ ID NO: 135 - DNA, 20 bp, Artificial Sequence
  Synthetic
caccggacctacttactcgc

SEQ ID NO: 136 - DNA, 20 bp, Artificial Sequence
  Synthetic
aaccccaaattggccgagat

SEQ ID NO: 137 - DNA, 20 bp, Artificial Sequence
  Synthetic
ggagaaaggagaggccgagc

SEQ ID NO: 138 - DNA, 22 bp, Artificial Sequence
  Synthetic
aaaagtaacccagtcagcaccg

SEQ ID NO: 139 - DNA, 20 bp, Artificial Sequence
  Synthetic
gtccggctcgcactttaaga

SEQ ID NO: 140 - DNA, 20 bp, Artificial Sequence
  Synthetic
ctgagaacgaggtcccttgc

SEQ ID NO: 141 - DNA, 20 bp, Artificial Sequence
  Synthetic
gtggtgtgagggggtttctg

SEQ ID NO: 142 - DNA, 20 bp, Artificial Sequence
  Synthetic
tacgaatagtccatgcccgc

SEQ ID NO: 143 - DNA, 19 bp, Artificial Sequence
  Synthetic
aggatgcatgggctgaacc

SEQ ID NO: 144 - DNA, 20 bp, Artificial Sequence
  Synthetic
ttgtagcagctcggacaagg

SEQ ID NO: 145 - DNA, 20 bp, Artificial Sequence
  Synthetic
caggccaaagtcacagcaac

SEQ ID NO: 146 - DNA, 20 bp, Artificial Sequence
  Synthetic
cgatccgagcagcactaaca

SEQ ID NO: 147 - DNA, 18 bp, Artificial Sequence
  Synthetic
gcaaaggacgagcgcaag

SEQ ID NO: 148 - DNA, 20 bp, Artificial Sequence
  Synthetic
cttgtagttggggtggtcgc

SEQ ID NO: 149 - DNA, 20 bp, Artificial Sequence
  Synthetic
agcggaggaggttttcagtg

SEQ ID NO: 150 - DNA, 20 bp, Artificial Sequence
  Synthetic
ttccattcggtctcgccaaa

SEQ ID NO: 151 - DNA, 20 bp, Artificial Sequence
  Synthetic
caggccaaagtcacagcaac

SEQ ID NO: 152 - DNA, 20 bp, Artificial Sequence
  Synthetic
cgatccgagcagcactaaca

SEQ ID NO: 153 - DNA, 52 bp, Artificial Sequence; identical to SEQ ID NO: 30
  Synthetic

SEQ ID NO: 154 - DNA, 51 bp, Artificial Sequence; identical to SEQ ID NO: 31
  Synthetic
  misc_feature (25)..(30): n is independently a or c or g or t

SEQ ID NO: 155 - DNA, 43 bp, Artificial Sequence; identical to SEQ ID NO: 32
  Synthetic

SEQ ID NO: 156 - DNA, 50 bp, Artificial Sequence
  Synthetic
gttgataacggactagccttattttaacttgctatttctagctctaaaac

SEQ ID NO: 157 - DNA, 38 bp, Artificial Sequence; identical to SEQ ID NO: 33
  Synthetic

SEQ ID NO: 158 - DNA, 83 bp, Artificial Sequence
  Synthetic
gttttagagctagaaatagcaagttaaaataaggctagtccgttatcaacttgaaaaagtggcaccgagtcggtgcttttttt

SEQ ID NO: 159 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 160 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

(19) United States
(12) Patent Application Publication
(10) Pub. No.: US 2022/0307015 A1
(43) Pub. Date: Sep. 29, 2022

(54) COMPOSITIONS AND METHODS FOR IDENTIFYING REGULATORS OF CELL TYPE FATE SPECIFICATION

(71) Applicant: Duke University, Durham, NC (US)

(72) Inventors: Charles A. Gersbach, Chapel Hill, NC (US); Joshua B. Black, Durham, NC (US); Jennifer Kwon, Durham, NC (US); Shaunak Adkar, Durham, NC (US)

(21) Appl. No.: 17/636,750

(22) PCT Filed: Aug. 19, 2020
(86) PCT No.: PCT/US20/47083

§ 371 Date: Feb. 18, 2022

Related U.S. Application Data
(60) Provisional application No. 62/888,922, filed on Aug. 19, 2019.
(60) Provisional application No. 62/889,361, filed on Aug. 20, 2019.
(60) Provisional application No. 62/961,084, filed on Jan. 14, 2020.

(51) Int. Cl. C12N 15/10; C07K 14/47; C12N 15/11; C12N 9/22; C12N 5/0793

(52) U.S. Cl. CPC C12N 15/1086; C07K 14/4705; C12N 15/11; C12N 9/22; C12N 5/0619; C12N 2310/20; C12N 2800/80; C12N 2501/60; C12N 2506/45; A61K 38/00

(57) ABSTRACT

Disclosed herein are compositions, methods, and systems for selecting a polynucleotide for activity as a neuronal-specific transcription factor. The system may include a polynucleotide encoding a reporter protein and a pan-neuronal marker, a Gas protein, and a library of guide RNAs (gRNAs) targeting putative transcription factors. Further provided are methods of screening for a neuronal-specific transcription factor.

### CROSS-REFERENCE TO RELATED APPLICATIONS

This application claims priority to U.S. Provisional Patent Application No. 62/888,922, filed Aug. 19, 2019, U.S. Provisional Patent Application No. 62/889,361, filed Aug. 20, 2019, and U.S. Provisional Patent Application No. 62/961,084, filed Jan. 14, 2020, each of which is incorporated herein by reference in its entirety.

### STATEMENT REGARDING FEDERALLY SPONSORED RESEARCH

This invention was made with government support under grants R21NS103007, DP2OD008586, R01DA036865, F31NS105419, and T32GM008555 awarded by the National Institutes of Health, and grant EFMA-1830957 awarded by the National Science Foundation. The government has certain rights in the invention.

### FIELD

This disclosure relates to DNA targeting compositions, such as CRISPRiCas9 compositions, and methods for identifying regulators of cell type fate specification.

### INTRODUCTION

The advent of methods to reprogram cell fate has revolutionized regenerative medicine, disease modeling, and cell therapy. Given the growing evidence defining specific neuronal subtypes as origins for neurological disease, the ability to generate these subtypes in vitro may facilitate the study and treatment of these complex diseases. Some current approaches to cell reprogramming overexpress transcription factors (TFs) to rewire the transcriptional programs of the starting cell. While this approach has succeeded in generating clinically relevant cell types, still relatively few cell types have been reprogrammed in this way. Efforts have been made to catalog the set of all putative human transcription factors and to define their tissue-specific expression, however, relatively few TFs have been empirically validated for a role in cell-fate specification. Further, the selection of fate-determining TFs for cell reprogramming applications often relies on approaches that evaluate a small subset of TFs or that use computational models to predict optimal TF combinations. Current strategies to develop new cell reprogramming protocols using TFs are slow, inefficient, and laborious. Previous studies have predominantly been in mice, yet the progression from mouse to human cell reprogramming is nontrivial. There are inherent differences in the plasticity of mouse cells versus human cells, Mouse cells are commonly more amenable to reprogramming, often obtaining higher efficiencies of conversion and shortened time to maturation, Consequently, human cells often require additional cofactors or entirely distinct protocols in order to achieve comparable conversion outcomes to their mouse counterparts. Given that the diversity of neuronal cell types in the human brain is likely programmed by a diversity of TFs, there remains a need for continued development of high-throughput approaches to systematically profile the causal role of TFs in directing neuronal cell-type identity, in particular, those that correlate well to humans.

### SUMMARY

In an aspect the disclosure relates to a polynucleotide that may encode: (1) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; or (2) a first neuronal-specific transcription factor selected from NGN3 and ASCL1, or a combination thereof; and a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1. FOSL1, PAX5, KLF3; (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7; (iv) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (v) HES2, SREBF1, CIC, WHSC1, UDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791; (vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1. ZNF160, ETV5, MYBL1, NOTO, DPF1, MECOM, GLIS3, KLF3, TBX22, ESX1, ZNF337. ZFP36L2, ELMSAN1, ZNF618, ZNF296, ZNF318, ZNF570, ZNF497, ZFP36L1, HES5, BMP2, CRAMP1 L, ZNF821, KMT2A, HES3, and BSX.

In a further aspect the disclosure relates to a system for increasing expression of a neuronal-specific gene, the system may comprise: (a) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, H1C1, SOX3, FOXJ1, SOX10, KLF6, ASCL1, and PLAGL2; or (b) a first gRNA targeting a first neuronal-specific transcription factor selected from NGN3 and ASCL1, or a combination thereof; and a second gRNA targeting a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3; (iii) RUNX3, PRDM1, KLF6, PAX2, RFX3, SOX10, GATA1, KLF5, KLFI, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, RF4, ASCLI, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7; (iv) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (v) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HEST, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, Z1M2, ZNF579, BMP2, CRAMP1L, TOX3, FEZF2, HES3, ZNF791; (vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOXS, ETS1, SKIL, BARHL2, SOX13, ERG, GRHL3, ZNF281, ELF3, HESX1, KLF15, PITX2, PTFIA, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1 L, ZNF821, KMT2A, HES3, and BSX; and a Cas protein or a fusion protein. In some embodiments, the fusion protein may comprise two heterologous polypeptide domains, wherein the first polypeptide domain comprises a Cas protein, a zinc finger protein, or a TALE protein, and the second polypeptide domain has an activity selected from transcription activation activity, transcription repression activity, transcription release factor activity, histone modification activity, nuclease activity, nucleic acid association activity, methylase activity, and demethylase activity. In some embodiments, the second neuronal-specific transcription factor is selected from LHX8, LHX6, E2F7, RUNX3, FOXH1, SOX2, HMX2, NKX2-2, HES3, and ZFP36L1. In some embodiments, the second neuronal-specific transcription factor may be selected from LHX8, LHX6, E2F7, RUNX3, FOXH1, SOX2, HMX2, and NKX2-2. In some embodiments, the second neuronal-specific transcription factor may be selected from HES3 and ZFP36L1. In some embodiments, the second neuronal-specific transcription factor may be selected from: (i) NEUROG3, SOX4, SOX9, KLF4, NR5A1, NEUROD1, SOX17, SMAD1, ATOH1, INSM1, NEUROG1, SOX18, RFX4, KLF7, SPB, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3, (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEURODI, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7, and wherein the second polypeptide domain has transcription activation activity. In some embodiments, the fusion protein may comprise ^{VP64}dCas9^{VP64} or dCas9-p300. In some embodiments, the second neuronal-specific transcription factor may be selected from: (i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HEST, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HESS, ZIM2, ZNF579, BMP2, CRAMP1L, TOX3, FEZF2, HES3, ZNF791; (iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L, ZNF821, KMT2A, HES3, and BSX, and wherein the second polypeptide domain has transcription repression activity. In some embodiments, the fusion protein may comprise dCas9-KRAB, In some embodiments, the first gRNA and the second gRNA each individually may comprise a 12-22 base pair complementary polynucleotide sequence of the target DNA sequence followed by a protospacer-adjacent motif, and optionally wherein the gRNA binds and targets and/or comprises a polynucleotide comprising a sequence selected from SEQ ID NOs: 38-87, and optionally wherein the first and/or second gRNA comprises a crRNA, a tracrRNA, or a combination thereof.

Another aspect of the disclosure provides an isolated polynucleotide that may encode the system as detailed herein.

Another aspect of the disclosure provides a vector that may comprise the isolated polynucleotide of as detailed herein.

In another aspect, the disclosure relates to a cell that may comprise the isolated polynucleotide as detailed herein or the vector as detailed herein.

In a further aspect the disclosure relates to a method of increasing maturation of a stem cell-derived neuron. The method may comprise: (a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SF8, OVOL1, NEUROG2, ERF, PRDM1, OLIG3, HIC1, SOX3, FOXJ1, SOX10, KLF6, ASCL1, and PLAGL2, or (b) increasing in the stem cell the level of a first neuronal-specific transcription factor selected from NGN3 and ASCL1, or a combination thereof; and increasing in the stem cell the level of a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLlG3, H1C1, SOX3, FOXJ1, SOX10, KLF6, ASCLI, and PLAGL2; (ii) PRDM1, LHX6, NEUROG3, PAX8, SOX3, KLF4, FLI1, FOXH1, FEV, SOX17, FOS, INSM1, SOX2, WT1, SOX18. ZNF670, LHX8, OVOL1, E2F7, AFF1, HMX2, MAZ, RARA, PROP1. FOSL1, PAX5, KLF3; (iii) RUNX3, PRDM1, KLF6, PAX2, RFX3. SOX10, GATA1. KLF5, KLF1, ERF, LHX6, PHOX2B, NANOG, NR5A2, ETV3, NEUROG3, SOX4. SOX9, PAXB, IRF5, CDX4, RARA, BHLHE40, SOX3, KLF4, NR5A1, IRF4, ASCL1, GATA6, SP1B, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

Another aspect of the disclosure provides a method of increasing maturation of a stem cell-derived neuron. The method may comprise: increasing in the stem cell the level of a first neuronal-specific transcription factor selected from NGN3 and ASCL1, or a combination thereof; and decreasing in the stem cell the level of a second neuronal-specific transcription factor selected from: (i) Z102, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VDR, HES1, ID2, TCF21, SNAI1, RREB1, GCM2, 1RF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HESS, Z1M2, ZNF579, BMP2, CRAMP1L, TOX3, FEZF2, HES3, ZNF791; (iii) ETV1, Z1C2. GSC2, CIC, GRHL2, REST, TFAP2C, SALL1. NFKB1, ELF2, HES1, MYB, KLF12. VSX2, NFE2, SNAI1, TRERF1, RREB1, IRF1, IRF3, KLF2, MYOD1, SOX15, BARX1, GRHL1, SOX5. ETS1, SKIL, BARHL2, SOX13, ERG, GRHL3, ZNF281. ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L, ZNF821, KMT2A, HES3, and BSX.

Another aspect of the disclosure provides a method of increasing the conversion of a stem cell to a neuron. The method may comprise: (a) increasing in the stern cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2, or (b) increasing in the stem cell the level of a first neuronal-specific transcription factor selected from NGN3 and ASCLI, or a combination thereof; and increasing in the stem cell the level of a second neuronal-specific transcription factor selected from: (i) NEUROG3, SOX4, SOX9, KLF4, NR5A1, NEUROD1, SOX17, SMAD1, ATOH1, INSM1, NEUROG1, SOX18. RFX4, KLF7, SP8, OVOL1, NEUROG2, ERF, PRDM1, OLIG3, H1C1, SOX3, FOXJ1, SOX10, KLF6, ASCLI, and PLAGL2; (ii) PRDM1, LHX6, NEUROG3, PAXB, SOX3, KLF4, FLI1. FOXH1, FEV, SOX17, FOS. INSM1. SOX2, WT1, SOX18, ZNF670, LHX8, OVOL1, E2F7, AFF1, HMX2, MAZ, RARA, PROP1, FOSL1, PAX5, KLF3, (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8,1RF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

Another aspect of the disclosure provides a method of increasing the conversion of a stem cell to a neuron. The method may comprise: increasing in the stem cell the level of a first neuronal-specific transcription factor selected from NGN3 and ASCL1, or a combination thereof; and decreasing in the stem cell the level of a second neuronal-specific transcription factor selected from: (i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, C1C, VVHSC1, VDR, HES1,1D2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, Z105, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791, (iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX.

Another aspect of the disclosure relates to a method of treating a subject in need thereof. The method may comprise: (a) increasing in a stern cell in the subject the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2, or (b) increasing in a stem cell in the subject the level of a first neuronal-specific transcription factor selected from NGN3 and ASCL1, or a combination thereof; and increasing in a stern cell in the subject the level of a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3: (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCLI, GATA6, SPIB, THRB, FOXH1, NEUROD1, SOX17, CDX2, ZEB2, RARG,INSM1, FOSL1, NEUROG1, SOX1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

Another aspect of the disclosure provides a method of treating a subject in need thereof. The method may comprise: increasing in a stern cell in the subject the level of a first neuronal-specific transcription factor selected from NGN3 and ASCL1, or a combination thereof; and decreasing in a stem cell in the subject the level of a second neuronal-specific transcription factor selected from: (i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, 2105, ZNF710, ZNF697, ZFP36L2, ELMSAN1, ZNF296, ZNF318, ZNF570, ZNF683, ZFP36L1, HES4, ZNF777, HESS, ZlM2, ZNF579, BMP2, CRAMP1L, TOX3, FEZF2, HES3, ZNF791; (iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSANI, ZNF618, ZNF296, ZNF318, ZNF570, ZNF497, ZFP36L1, HESS, BMP2, CRAMP1 L, ZNF821, KMT2A, HES3, and BSX. In some embodiments, increasing the level of the first neuronal-specific transcription factor may comprise at least one of: (a) administering to the stem cell a polynucleotide encoding the first neuronal-specific transcription factor; (b) administering to the stem cell a polypeptide comprising the first neuronal-specific transcription factor; and (c) administering to the stem cell a fusion protein, wherein the fusion protein comprises two heterologous polypeptide domains, wherein the first polypeptide domain comprises a Cas protein, a zinc finger protein targeting the first neuronal-specific transcription factor, or a TALE protein targeting the first neuronal-specific transcription factor, and the second polypeptide domain has transcription activation activity, and wherein a gRNA targeting the first neuronal-specific transcription factor is additionally administered to the stem cell when the first polypeptide domain comprises a Cas protein. In some embodiments, increasing the level of the second neuronal-specific transcription factor may comprise at least one of: (a) administering to the stem cell a polynucleotide encoding the second neuronal-specific transcription factor; (b) administering to the stem cell a polypeptide comprising the second neuronal-specific transcription factor; and (c) administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription activation activity, and wherein a gRNA targeting the second neuronal-specific transcription factor is additionally administered to the stem cell when the first polypeptide domain comprises a Cas protein. In some embodiments, decreasing the level of the second neuronal-specific transcription factor may comprise administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription repression activity, and wherein a gRNA targeting the second neuronal-specific transcription factor is additionally administered to the stem cell when the first polypeptide domain comprises a Cas protein. In some embodiments, the stem cell may be directly converted to a neuron without a pluripotent stage. In some embodiments, the stem cell may be a pluripotent stem cell, an induced pluripotent stem cell, or an embryonic stem cell.

Another aspect of the disclosure provides a system for selecting a polynucleotide for activity as a cell type-specific transcription factor. The system may comprise: a polynucleotide encoding a reporter protein and a cell type marker; a fusion protein, wherein the fusion protein comprises two heterologous polypeptide domains, wherein the first polypeptide domain comprises a Cas protein, and the second polypeptide domain has transcription activation activity; and a library of guide RNAs (gRNAs), each gRNA targeting a different putative cell type-specific transcription factor. In some embodiments, the cell-type specific transcription factor may be a neuronal-specific transcription factor, wherein the cell type marker is a neuronal marker, and wherein the neuronal marker comprises TUBB3. In some embodiments, the cell-type specific transcription factor may be a muscle-specific transcription factor, wherein the cell type marker is a myogenic marker, and wherein the myogenic marker comprises PAX7. In some embodiments, the cell-type specific transcription factor may be a chondrocyte-specific transcription factor, wherein the cell type marker is a collagen marker, and wherein the collagen marker comprises COL2A1. In some embodiments, the reporter protein may comprise mCherry.

Another aspect of the disclosure provides an isolated polynucleotide sequence that may encode the system as detailed herein.

Another aspect of the disclosure provides a vector that may comprise the isolated polynucleotide sequence as detailed herein.

Another aspect of the disclosure provides a cell that may comprise the system as detailed herein, the isolated polynucleotide sequence as detailed herein, or the vector as detailed herein, or a combination thereof.

Another aspect of the disclosure provides a method of screening for a cell type-specific transcription factor. The method may comprise: transducing a population of cells with the system as detailed herein at a multiplicity of infection (MOI) of about 0.2, such that a majority of the cells each independently includes one gRNA and targets one putative transcription factor; determining a level of expression of the reporter protein in each cell; determining a level of the gRNA in each cell having a high expression of the reporter protein. In some embodiments, high expression of the reporter protein may be defined as being in the top 5% among the population of cells; and selecting the putative transcription factor as a cell-type-specific transcription factor when the putative transcription factor corresponds to at least two gRNAs enriched in the cell having a high expression of the reporter protein.

Another aspect of the disclosure provides a method of screening for a pair of cell-type-specific transcription factors. The method may comprise: transducing a population of cells with the system as detailed herein at a multiplicity of infection (MOI) of about 0.2, such that a majority of the cells each independently includes two gRNAs and targets two putative transcription factors; determining a level of expression of the reporter protein in each cell; determining a level of the two gRNAs in each cell having a high expression of the reporter protein. In some embodiments, high expression of the reporter protein may be defined as being in the top 5% among the population of cells; and selecting the two putative transcription factors as a pair of cell type-specific transcription factors when the putative transcription factors correspond to at least two gRNAs enriched in the cell having a high expression of the reporter protein. In some embodiments, the level of expression of the reporter protein in each cell may be determined after about four days from transduction. In some embodiments, the level of expression of the reporter protein in each cell may be determined by flow cytometry. In some embodiments, the level of the gRNA in each cell having a high expression of the reporter protein may be determined by deep sequencing. In some embodiments, the gRNA may increase the expression of the reporter protein in the cell by about 2-50% relative to a non-targeting gRNA.

Another aspect of the disclosure provides a polynucleotide encoding a muscle-specific transcription factor selected from TWIST1, PAX3, MYOD, MYOG, SOX9, SOX10, and DMRT1.

Another aspect of the disclosure provides a system for increasing expression of a muscle-specific gene. The system may comprise: (a) a muscle-specific transcription factor selected from TWIST1, PAX3, MYOD, MYOG, SOX9, SOX10, and DMRT1; or (b) a fusion protein, wherein the fusion protein comprises two heterologous polypeptide domains. In some embodiments, the first polypeptide domain may comprise a Gas protein, a zinc finger protein targeting a muscle-specific transcription factor selected from TWIST1, PAX3, MYOD, MYOG, SOX9, SOX10, and DMRT1, ora TALE protein targeting a muscle-specific transcription factor selected from TWIST1, PAX3, MYOD, MYOG, SOX9, SOX10, and DMRT1, wherein the second polypeptide domain has an activity selected from transcription activation activity, transcription release factor activity, histone modification activity, nucleic acid association activity, methylase activity, and demethylase activity, and wherein the system further includes a gRNA targeting a muscle-specific transcription factor selected from TWIST1, PAX3, MYOD, MYOG, SOX9, SOX10, and DMRT1 when the first polypeptide domain comprises a Cas protein. In some embodiments, the fusion protein may comprise ^{VP64}dCas9^{VP64} or dCas9-p300.

Another aspect of the disclosure provides an isolated polynucleotide that may encode the system as detailed herein.

Another aspect of the disclosure provides a vector that may comprise the isolated polynucleotide as detailed herein.

Another aspect of the disclosure provides a cell that may comprise the isolated polynucleotide as detailed herein or the vector as detailed herein.

Another aspect of the disclosure provides a method of increasing differentiation of a stem cell into a myoblast. The method may comprise: increasing in the stem cell the level of a muscle-specific transcription factor selected from TWIST1, PAX3, MYOD, MYOG, SOX9, SOX10, and DMRT1.

Another aspect of the disclosure provides a method of treating a subject in need thereof. The method may comprise: increasing in a stem cell from the subject the level of a muscle-specific transcription factor selected from TWIST1, PAX3, MYOD, MYOG, SOX9, SOX10, and DMRT1. In some embodiments, increasing the level of the muscle-specific transcription factor may comprise at least one of: (a) administering to the stem cell a polynucleotide encoding the muscle-specific transcription factor; (b) administering to the stem cell a polypeptide comprising the muscle-specific transcription factor; and (c) administering to the stem cell a fusion protein, wherein the fusion protein comprises two heterologous polypeptide domains, wherein the first polypeptide domain comprises a Cas protein, a zinc finger protein targeting the muscle-specific transcription factor, or a TALE protein targeting the muscle-specific transcription factor, wherein the second polypeptide domain has transcription activation activity, and wherein a gRNA targeting the muscle-specific transcription factor is additionally administered when the first polypeptide domain comprises a Cas protein.

The disclosure provides for other aspects and embodiments that will be apparent in light of the following detailed description and accompanying figures.

### BRIEF DESCRIPTION OF THE DRAWINGS

FIG. 1A-FIG. 1G. A high-throughput CRISPRa screen identifies candidate neurogenic transcription factors. (FIG. 1A) Schematic representation of a CRISPRa screen for neuronal-fate determining transcription factors in human pluripotent stem cells. A ^{VP64}dCas9^{VP64} TUBB3-2A-mCherry reporter cell line was transduced with the CAS-TF pooled lentiviral library at an MOI of 0.2 and sorted for mCherry expression via FACS, gRNA abundance in each cell bin was measured by deep sequencing, and depleted or enriched gRNAs were identified by differential expression analysis, (FIG. 1B) The CAS-TF gRNA library was extracted from a previous genome-wide CRISPRa library (Horlbeck, 2016, Compact and highly active next-generation libraries. eLife) and consists of 8,505 gRNAs targeting 1496 putative transcription factors. (FIG. 1C) TUBB3-2A-mCherry cells were sorted for the highest and lowest 5% expressing cells based on mCherry signal. A bulk unsorted population of cells was also sampled to establish the baseline gRNA distribution. (FIG. 1D) Differential expression analysis of normalized gRNA counts between the mCherry-High and Unsorted cell populations. Red data points indicate FDR<0.01 by differential DESeq2 analysis (n=3 biological replicates). Blue data points indicate a set of 100 scrambled non-targeting gRNAs. (FIG. 1E) Analysis of TF family type across the 17 TFs identified in the CAS-TF screen. (FIG. 1F) Comparison of average gene expression across multiple developmental time points and anatomical brain regions for the 17 IFs identified in the CAS-TF screen and three random sets of 17 TFs. (FIG. 1G) The fold change in gRNA abundance from differential expression analysis between mCherry-High and mCherry-Low cell populations for all five gRNAs from three known proneural TFs compared to a random selection of five scrambled gRNAs. See also FIG. 7A-FIG. 7D.

FIG. 2A-FIG. 2F. Many candidate factors generate neuronal cells from pluripotent stem cells. (FIG. 2A) Validations of 17 factors for TUBB3-2A-mCherry expression four days after transduction of gRNAs (*p<0,05 by global one-way ANOVA with Dunnett's post hoc test comparing all groups to Scrambled 1, gating set to 1% positive for Scrambled gRNAs, n=3 biological replicates, error bars represent SEM). (FIG. 2B) The relationship between TUBB3-2A-mCherry expression assessed by individual validations and the fold change in gRNA abundance from differential expression analysis of the library selection for all five gRNAs from ATOH1 and NR5A1. (FIG. 2C) Validations of 17 factors for the induction of the pan-neuronal markers NCAM (top) and MAP2 (bottom) four days after transduction of gRNAs (*p<0.05 by global one-way ANOVA with Dunnett's post hoc test comparing all groups to Scrambled 1, n=3 biological replicates, error bars represent SEM). (FIG. 2D) Immunofluorescence staining of iPSCs assessing TUBB3 expression four days after transduction with tetracycline-inducible lentiviral vectors carrying cDNAs encoding the indicated factors, or with a M2rtTA-only negative control. Scale bar, 50 μm. (FIG. 2E) Immunofluorescence staining of iPSCs assessing MAP2 expression with the indicated factors after extended co-culture with astrocytes. Scale bar, 50 μm. (FIG. 2F) Immunofluorescence staining of H9 hESCs assessing TUBB3 expression four days after transduction of the indicated factors. See also FIG. 8A-FIG. 8C, FIG. 9A-FIG. 9D, and FIG. 10A-FIG. 10E.

FIG. 3A-FIG. 3G. Combinatorial gRNA screens identify cofactors of neuronal differentiation. (FIG. 3A) Schematic representation of combinatorial CRISPRa screens for neuronal-fate determining transcription factors in human pluripotent stem cells. A dual gRNA expression vector was used to co-express a neurogenic factor with the CAS-TF gRNA library. Two independent screens were performed with sgASCL1 and sgNGN3. (FIG. 3B) A volcano plot of significance (P value) versus fold-change in gRNA abundance based on differential DESeq2 analysis between mCherry-High and Unsorted cell populations for the sgNGN3 paired screen. Red data points indicate FDR<0.001 (n=3 biological replicates). Blue data points indicate a set of 100 scrambled non-targeting gRNAs. (FIG. 3C) The fold-change in gRNA abundance for the sgASCL1 versus sgNGN3 paired screens for all positively enriched gRNAs across both screens. (FIG. 3D) Analysis of TF family type and basal expression level in pluripotent stem cells for the positive hits from both paired screens. (FIG. 3E) The fold-change in gRNA abundance for a set of TFs predicted to have no activity individually and synergistic activity in the sgASCL1 and sgNGN3 paired screens. Validations of TF cofactors for sgNGN3 with TUBB3-2A-mCherry (FIG. 3F) and sgASCL1 with NCAM staining (FIG. 3G), (*p<0.05 by global one-way ANOVA with Dunnett's post hoc test comparing all groups to Scrambled 1, n=3 biological replicates, error bars represent SEM), See also FIG. 11A-FIG. 11B and FIG. 12A-FIG. 12D.

FIG. 4A-FIG. 4F. Transcriptional diversity of neurons generated by single transcription factors. (FIG. 4A) Differentially up-regulated genes detected in ATOH1 and NEUROG3-derived neurons (FDR<0.01 & log2(fold-change)>1), (FIG. 4B) Enriched gene ontology (GO) terms for the set of 2846 genes shared and up-regulated between ATOH1 and NEUROG3, (FIG. 4C) Expression level (log2(TPM+1)) of a set of pan-neuronal genes across all replicate samples analyzed. (FIG. 4D) Comparison of all detected genes between ATOH1 and NEUROG3-derived neurons. Red and blue circles represent genes differentially expressed with either NEUROG3 or ATOH1, respectively. (FIG. 4E) GO term analysis for markers up-regulated uniquely with either NEUROG3 or ATOH1. (FIG. 4F) Expression level (log2(TPM+1)) and corresponding z-scores for a set of dopaminergic and glutarnatergic markers.

FIG. 5A-FIG. 5N, Transcriptional and functional maturation of neurons generated with pairs of transcription factors. (FIG. 5A) Differentially up-regulated genes detected in neurons derived from pairs of IFs (FDR<0.01 & log2(fold-change)>1), (FIG. 5B) GO terms enriched in the set of differentially up-regulated genes with pairs of IFs compared to NEUROG3 alone. Up-regulation of (FIG. 5C) NTRK3 and (FIG. 5D) CDKN1A with the addition of RUNX3 or E2F7, respectively. (FIG. 5E) SynGO terms for the set of genes differentially up-regulated with the addition of LHX8. (FIG. 5F) Expression level (bottom; log2(fold-change); top: log2(TPM+1)) fora set of synaptic markers. Average values of membrane properties including (FIG. 5G) resting membrane potential (Vrest), (FIG. 5H) input resistance (Rₘ) and (FIG. 51) membrane capacitance (Cₘ) for day 7 neurons generated with NEUROG3 alone or in combination with LHX8. Average values of action potential properties including (FIG. 5J) action potential threshold (AP_{threshold}), (FIG. 5K) action potential height (AP_{height}) and (FIG. 5L) action potential half-width (AP_{half-width}) for day 7 neurons generated with NEUROG3 alone or in combination with LHX8. (FIG. 5M) Average number of action potentials generated with respect to amplitude of injected current (*p<0.05 two-way ANOVA). (FIG. 5N) Example traces 01 cells with failed (left), single (middle), or multiple (right) action potentials. The corresponding pie chart represents the total fraction of cells analyzed that failed to generate an AP (dark shade), generated a single AP (medium shade), or generated multiple APs (light shade) in response to a single depolarization current injection. For FIG. 5G to FIG. 5L: ns, not significant; *p<0.05 unpaired t-test (if data passes normality; alpha=0.05) or Mann-Whitney test (if data fails normality; alpha=0.05); n=19 cells for NEUROG3 alone; n=22 cells for NEUROG3 LHX8.

FIG. 6A-FIG. 6I. Combinatorial gRNA screens identify negative regulators of neuronal differentiation. (FIG. 6A) The fold change in gRNA abundance for the sgASCL1 versus sgNGN3 paired screens for all negatively enriched gRNAs across both screens. (FIG. 6B) Validations for a subset of TFs assessing percent TUBB3-2A-mCherry positive cells and (FIG. 6C) expression of the pan-neuronal marker NCAM (*p<0.05 by global one-way ANOVA with Dunnett's post hoc test comparing all groups to the sgNGN3+ Scrambled gRNA condition, n=3 biological replicates, error bars represent SEM). (FIG. 6D) Validations of the same negative regulators in H9 hESCs. (FIG. 6E) Comparison of gRNA effects on neuronal differentiation in iPSCs versus ESCs. (FIG. 6F) Schematic representation of orthogonal gene activation and repression. (FIG. 6G) Relative expression of the top 100 variable genes quantified by z-score between all three groups tested. (FIG. 6H) GO terms enriched in the set of differentially expressed genes in sgNGN3-derived neurons with ZFP36L1 knockdown. (FIG. 61) Example set of differentially expressed genes associated with neuronal differentiation and morphological development. See also FIG. 13A-FIG. 13C and FIG. 14A-FIG. 14D.

FIG. 7A-FIG. 7D. Generation and characterization of a TUBB3-2A-mCherry reporter cell line. (FIG. 7A) Schematic representation of the knock-in of a P2A-mCherry cassette into exon four of TUBB3 in a human pluripotent stem cell line using Cas9 nuclease and a donor template. (FIG. 7B) Targeted activation of endogenous NEUROG2 in pluripotent stem cells with ^{VP64}dCas9^{VP64} and a set of four gRNAs targeting the NEUROG2 promoter. Expression of NCAM (middle) and MAP2 (right) with targeted activation of NEUROG2 (n=2 biological replicates). (FIG. 7C) TUBB3-2A-mCherry expression by flow cytometry with targeted activation of NEUROG2 with ^{VP64}dCas9^{VP64} and a set of four gRNAs targeting the promoter. (FIG. 7D) TUBB3 and MAP2 expression in TUBB3-2A-mCherry cells sorted for the highest and lowest mCherry expression after activation of NEUROG2 with ^{VP64}dCas9^{VP64} and gRNAs (n=1 biological replicate).

FIG. 8A-FIG. 8C. Validations of TFs with a single enriched gRNA. (FIG. 8A) A ranked list of fold change in gRNA abundance between mCherry-High versus mCherry-Low expressing cells in the single factor CAS-TF screen. ASCL1, ATOH7, and ATOH8 all have a single gRNA significantly enriched. (FIG. 8B) Individual validations of sgASCL1, sgATOH7, and sgATOH8 for (FIG. 8B) percent TUBB3-2A-mCherry expression and (FIG. 8C) MAP2 (left) and NCAM (right) expression four days after gRNA transduction (*p<0.05 by global one-way ANOVA with Dunnett's post hoc test comparing all groups to a scrambled gRNA, n=3 biological replicates, error bars represent SEM).

FIG. 9A-FIG. 9D. Endogenous induction of TFs with ^{VP64}dCas9^{VP64}. (FIG. 9A) Fold induction of a subset of 17 TFs enriched in the single factor CAS-TF screen with ^{VP64}dCas9^{VP64} and the top enriched gRNA (fold change relative to a scrambled gRNA, n=2 biological replicates). (FIG. 9B) Relation between the fold induction of each TF and the basal expression of that TF relative to GAPDH expression. (FIG. 9C) Comparison of gRNA enrichment from the single factor CAS-TF screen for two NEUROG2 gRNAs. (FIG. 9D) Validation of these two NEUROG2 gRNAs for IF induction and expression of downstream neuronal markers (*p<0.05 by global one-way ANOVA with a Tukey post hoc test comparing the two NEUROG2 gRNAs, n=3 biological replicates, error bars represent SEM).

FIG. 10A-FIG. 10E. CAS-TF sub-library gRNA screen. (FIG. 10A) Schematic representation of the CRISPRa sub-library screen for neuronal-fate determining transcription factors in human pluripotent stem cells. A ^{VP64}dCas9^{VP64} TUBB3-2A-mCherry reporter cell line was transduced with the CAS-TF pooled lentiviral library at an MOI of 0.2 and sorted for mCherry expression via FACS. gRNA abundance in each cell bin was measured by deep sequencing, and depleted or enriched gRNAs were identified by differential expression analysis. (FIG. 10B) The CAS-TF gRNA sub-library was extracted from several previous genome-wide CRISPRa library and consisted of 3,874 gRNAs targeting 109 putative transcription factors (˜33 gRNAs per gene), (FIG. 10C) Differential expression analysis of normalized gRNA counts between the rnCherry-High and mCherry-Low cell populations. Red data points indicate FDR <0.01 by differential DESeq2 analysis (n=3 biological replicates). (FIG. 10D) Ranked list of percent enriched gRNAs per gene. (FIG. 10E) Validations of 10 factors for TUBB3-2A-mCherry expression four days after transduction of gRNAs (n=2 biological replicates).

FIG. 11A-FIG. 11B. Paired gRNA screen with sgASCL1. A volcano plot of significance (P value) versus fold-change in gRNA abundance based on differential DESeq2 analysis between (FIG. 11A) mCherry-High vs. Unsorted and (FIG. 11B) mCherry-High vs. mCherry-Low cell populations for the sgASCL1 paired screen. Red data points indicate FDR<0.001 (n=3 biological replicates).

FIG. 12A-FIG. 12D. Comparisons of the single factor and paired CAS-TF screens. The fold change in gRNA abundance between mCherry-High and mCherry-Low expressing cells for the (FIG. 12A and FIG. 12B) sgNGN3 versus single factor CAS-TF screens for all positively (FIG. 12A) and negatively (FIG. 12B) enriched gRNAs across both screens and (FIG. 12C and FIG. 12D) sgASCL1 versus single factor CAS-TF screens for all positively (FIG. 12C) and negatively (FIG. 12D) enriched gRNAs across both screens.

FIG. 13A-FIG. 13C. Gene activation and repression with orthogonal CRISPR systems. (FIG. 13A) Targeted repression of ZFP36L1 and HES3 in pluripotent stem cells using dSaCas9^{KRAB}targeting the promoter with a single gRNA for seven days (*p<0.05 by two-tailed t-test, n=3 biological replicates, error bars represent SEM). Effects on differentiation with either sgNGN3 (FIG. 13B) or sgASLC1 (FIG. 13C) in ZFP36L1 and HES3 knockdown cell lines (*p<0.05 by global one-way ANOVA with Dunnett's post hoc test comparing all groups with either sgNGN3 or sgASCL1 to the Control cell line that received a scrambled non-targeting S. aureus gRNA, n=3 biological replicates, error bars represent SEM).

FIG. 14A-FIG. 14D, Genome-wide expression analysis with orthogonal CRISPR-based gene regulation. Differential expression analysis for sgNGN3-derived neurons with (FIG. 14A) HESS knockdown and (FIG. 14B) ZFP36L1 knockdown. Red data points indicate FDR<0.01 by differential expression analysis with DESeq2 (n=3 biological replicates). (FIG. 14C) Expression of the S. pyogenes gRNA target gene, NEUROG3, across the three conditions shown. (FIG. 14D) GFP expression on the S. pyogenes gRNA lentiviral vector was used as a proxy for transduction level and gRNA expression across the three conditions shown,

FIG. 15A-FIG. 15E. Generation and validation of a PAX7-2a-GFP reporter cell line in human ESCs. (FIG. 15A) PAX7 gene targeting strategy. A gRNA was designed to target the stop codon of PAX7, and a 2a-GFP donor cassette containing an excisable selection marker was designed for insertion via homologous recombination. (FIG. 158) PCR validation of clones with primers outside of the homology arms shows heterozygous insertion of the reporter cassette. (FIG. 15C) Sequencing of the 2.6 kb product confirms insertion of the 2a-GFP reporter cassette. (FIG. 15D) Targeting the PAX7 promoter of a single clone for activation via CRISPRa demonstrates a shift in GFP. (FIG. 15E) The top 15% and bottom 15% of GFP expressing cells correspond to high and low PAX7 mRNA expression, respectively.

FIG. 16A-FIG. 16E. A CRa-TF screen for upstream regulators of PAX7. (FIG. 16A) Schematic of CRa-TF screen. H9 Pax7-2a-GFP cells stably expressing ^{VP64}dCas9^{VP64} were transduced with the CRa-TF lentiviral library at an MOI of 0.2. Cells were selected and differentiated for 14 days with small molecules CHIRON99021 (CHIR) and bFGF. Top 10% and bottom 10% of GFP expressing cells were sorted and DNA was deep sequenced to recover gRNAs. (FIG. 16B) Histogram at day 14 of differentiation demonstrates a GFP+population emerging in three replicates of the CRa-TF screen compared to a no library control. (FIG. 16C) MA plot demonstrating significant gRNA hits (p<0.05) in the top 10% compared to unsorted cells. (FIG. 16D) Validation of individual gRNA hits demonstrating induction of PAX7. (FIG. 16E) cDNA delivery of hits also demonstrates induction of PAX7 (mean±SEM, n=3).

FIG. 17A-FIG. 17C. Combinatorial CRa-TF screen to identify PAX7 cofactors. (FIG. 17A) In a second version of the initial screen, the lentiviral construct was redesigned to include a PAX7-targeting gRNA. Lentivirus was transduced at an MOI of 0.2 such that each cell receives one copy of the PAX7 gRNA and a gRNA from the CRa-TF library. (FIG. 17B) Histogram at day 7 of differentiation demonstrates a shift in GFP in three replicates of the second CRa-TF screen compared to a no library control. (FIG. 17C) A venn diagram showing unique and overlapping significant (p<0.05) hits from both versions of the screen.

FIG. 18A-FIG. 18D. Validation of myogenic lineage induction by CRa-TF hits. (FIG. 18A) Schematic of validation by inducible expression of hits. H9 PAX7-2a-GFP expressing TetO-^{VP64}dCas^{VP64} was transduced with individual gRNA hits and rtTA3. Cells were differentiated for 28 days in the presence of dox, Terminal differentiation was induced by withdrawing dox for 14 days prior to analysis. (FIG. 18B) RNA analysis after terminal differentiation demonstrates increased PAX7 expression compared to a non-targeting gRNA control. (FIG. 18C) RNA analysis after terminal differentiation demonstrates increased MYOG expression compared to a non-targeting gRNA control (mean±SEM, n=3). (FIG. 18D) Images of the cells.

FIG. 19A-FIG. 19B. Generation and validation of a polyclonal transactivator line. (FIG. 19A) Schematic of ^{VP64}dCas9^{V46}-2A-blasticidin expression cassette. (FIG. 19B) Activation of endogenous NGN2 after transduction of NGN2.

FIG. 20A-FIG. 20C. TF-targeted gRNA screen to identify regulators of chondrogenesis. (FIG. 20A) Experimental schematic demonstrating generation of activator line in the reporter line and lentiviral packaging of gRNA library. Alter transduction of library and chondrogenic differentiation, GFP^{high} and GFP^{low} cells were sorted and gRNAs were recovered from both populations. Differential expression of gRNAs were compared using next-generation sequencing. (FIG. 20B) Histogram of GFP fluorescence after library transduction and chondrogenic differentiation. Gates show GFP^{high} and GFP^{low} sorted populations. (FIG. 20C) Volcano plot illustrating significantly enriched gRNAs in GFP^{high} and GFP^{low} populations (red) as well as gRNAs not meeting significance criteria but with high (>3) log2(fold change). See Appendix B for larger volcano plot.

FIG. 21A-FIG. 21C. Validation of SOX9 in context of directed differentiation. (FIG. 21A) Schematic of experimental design. Differentiation of reporter hiPSCs with SOX9 overexpression to sclerotorne, followed by flow cytometry at day 6. (FIG. 21B) Flow cytometry at day 6 of unmodified line compared to reporter line with (red) and without (black) SOX9 lentivirus. (FIG. 21C) Comparison of day 6 data with GFP fluorescence at day 21 (blue) of differentiation.

### DETAILED DESCRIPTION

Detailed herein are cell type-specific transcription factors and methods of using the same to increase expression of a cell type-specific gene, increase maturation of a stem cell-derived neuron, increase the conversion efficiency of a stem cell to a neuron, and treat a subject in need thereof, Further detailed herein is a high-throughput pooled CRISPR activation (CRISPRa) screen to map human cell-fate regulators and profile the contribution of putative human transcription factors for neuronal cell-fate specification of pluripotent stem cells. CRISPRa screens were used in a high-throughput approach to profile thousands of putative transcription factors in the human genome. CRISPR-based gRNA libraries are more easily designed and scaled, and are more amenable to testing combinatorial gene interactions and interrogating the non-coding genome than conventional methods. Using a reporter of neuronal commitment, the neurogenic activity of all transcription factors in human pluripotent stem cells was profiled. A single-factor screen was performed to identify master regulators of human neuronal fate, and many known and previously uncharacterized TFs were identified, Combinatorial screens were performed, and synergistic and antagonistic TF interactions that enhance or diminish neuronal differentiation were identified, respectively. TFs were uncovered that increase conversion efficiency, influence subtype specification, and improve maturation of in vitro-derived human neurons.

Collectively, this work highlights the utility of DNA targeting systems such as CRISPR-based technologies for regulating endogenous gene expression and provides a framework for identifying the causal role of cell-fate regulators in defining any cell type of interest. The set of candidate proneural transcription factors curated from the study detailed herein can serve as a resource for establishing protocols to generate every cell type in the human brain.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a nuclease target region that is recognized and bound by the nuclease.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e,g., from Analyse-it Software Ltd., Leeds, UK; StataCorp LP, College Station, Tex.; SAS Institute Inc., Cary, N.C.), The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Fusion protein” as used herein refers to a chimeric protein created through the translation of two or more joined genes that originally coded for separate proteins. The translation of the fusion gene results in a single polypeptide with functional properties derived from each of the original separate proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thyrnine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded, or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

“Sample” or “test sample” as used herein can mean any sample in which the presence and/or level of a target is to be detected or determined or any sample comprising a DNA targeting system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Spacers” and “spacer region” as used interchangeably herein refers to the region within a TALE or zinc finger target region that is between, but not a part of, the binding regions for two TALEsor zinc finger proteins,

“Subject” or “patient” as used herein can mean an animal that wants or is in need of the herein described compositions or methods. The subject may be a human or a non-human. The subject may be any vertebrate. The subject may be a mammal. The mammal may be a primate or a non-primate. The mammal can be a non-primate such as, for example, dog, cat, horse, cow, pig, mouse, rat, mouse, camel, llama, goat, rabbit, sheep, hamster, and guinea pig. The mammal can be a primate such as a human. The mammal can be a non-human primate such as, for example, monkey, cynomolgous monkey, rhesus monkey, chimpanzee, gorilla, orangutan, and gibbon, The subject may be of any age or stage of development, such as, for example, an adult, an adolescent, or an infant, The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids Of' nucleotides, respectively,

“Transcription activator-like effector” or “TALE” refers to a protein structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules may be arranged in any order to assemble an array that recognizes a defined sequence, A binding specificity of a TALE DNA-binding domain is determined by the RVD array followed by a single truncated repeat of 20 amino acids. “Repeat variable diresidue” or “RVD” refers to a pair of adjacent amino acid residues within a DNA recognition motif (also known as “RVD module”), which includes 33-35 amino acids, of a TALE DNA-binding domain, The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region A TALE DNA-binding domain may have 12 to 27 RVD modules, each of which contains an RVD and recognizes a single base pair of DNA, Specific RVDs have been identified that recognize each of the four possible DNA nucleotides (A, T, C, and G). Because the TALE DNA-binding domains are modular, repeats that recognize the four different DNA nucleotides may be linked together to recognize any particular' DNA sequence. These targeted DNA-binding domains may then be combined with catalytic domains to create functional enzymes, including artificial transcription factors, methyltransferases, integrases, nucleases, and recombinases.

“Target gene” as used herein refers to any nucleotide sequence encoding a known or putative gene product. The target gene may be a mutated gene involved in a genetic disease. In certain embodiments, the target gene is a gene encoding a transcription factor.

“Target region” as used herein refers to the region of the target gene to which the CRISPR/Cas9-based gene editing system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “treating,” when referring to protection of a subject from a disease, means suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a Cas9 protein and at least one gRNA molecule.

“Zinc finger” as used herein refers to a protein that recognizes and binds to DNA sequences. The zinc finger domain is the most common DNA-binding motif in the human proteome. A single zinc finger contains approximately 30 amino acids, and the domain typically functions by binding 3 consecutive base pairs of DNA via interactions of a single amino acid side chain per base pair.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. TRANSCRIPTION FACTOR

Provided herein are cell type-specific transcription factors. A transcription factor (TF) is a protein that controls the rate of transcription of genetic information from DNA to messenger RNA, by binding to a specific DNA sequence. TFs regulate genes to ensure they are expressed in the right cell at the right time and in the right amount throughout the life of the cell and the organism. TFs transmit complex patterns of intrinsic and extrinsic signals into dynamic gene expression programs that define cell-type identity. Groups of TFs may function in a coordinated fashion to direct, for example, cell division, cell growth, and cell death throughout life; cell migration and organization (body plan) during embryonic development; and intermittently in response to signals from outside the cell, such as a hormone, TFs may work alone or with other proteins in a complex, by, for example, promoting or blocking the recruitment of RNA polymerase. The TF may be specific for a particular cell type. The TF may be neuronal-specific. The TF may be muscle-specific. The TF may be chondrocyte-specific. The TF may be specific for any cell type, such as, for example, cells from a tissue selected from bone marrow, skin, skeletal muscle, fat tissue, and peripheral blood. The cells may be muscle cells (such as smooth muscle cells, skeletal muscle cells, and cardiac muscle cells, for example), epithelial cells, endothelial cells, urothelial cells, fibroblasts, hepatocytes, myoblasts, neurons, osteoblasts, osteoclasts, T-cells, keratinocyte cells, hair follicle cells, human umbilical vein endothelial cells (HUVEC), cord blood cells, neural progenitor cells, chondrocytes, chondroblasts, bile duct cells, pancreatic islet cells, thyroid cells, parathyroid cells, adrenal cells, hypothalamic cells, pituitary cells, ovarian cells, testicular cells, salivary gland cells, adipocytes, precursor cells, hematopoietic stem cells (HSC), mesenchymal stem cells (MSC) of adipose, mesenchymal stem cells (MSC) of bone marrow, oligodendrocytes, oligodendrocyte precursors, neutrophils, basophils, eosinophils, lymphocytes, monocytes, or cardiomyocytes. The TF may be a member of, for example, the C2H2 ZF, bHLH, or HMG/Sox DNA-binding domain families. The TF may be an activating TF (which activates or increases expression of a gene), or the TF may be a repressing TF (which represses or reduced the expression of a gene).

TFs may use a variety of mechanisms to regulate gene expression. For example, TFs may stabilize or block the binding of RNA polymerase to DNA. TFs may recruit coactivator or corepressor proteins to the transcription factor DNA complex. TFs may directly or indirectly catalyze the acetylation or deacetylation of historic proteins. Histone acetyltransferase (HAT) activity acetylates histone proteins, which weakens the association of DNA with histones, which may make the DNA more accessible to transcription, thereby up-regulating transcription. Histone deacetylase (HDAC) activity deacetylates histone proteins, which strengthens the association of DNA with histones, which may make the DNA less accessible to transcription, thereby down-regulating transcription. TFs may influence the three dimensional looping of DNA, which can in turn affect gene expression.

Provided herein are polynucleotides encoding at least one transcription factor, or the transcription factor polypeptides themselves. In some embodiments, the transcription factor is an endogenous transcription factor. “Endogenous” here refers to the copy of the gene that encodes the TF in its natural position in the subject's genome in chromosomal DNA. The transcription factor may direct expression of genes in neurons. The transcription factor may direct differentiation of a cell into a neuron. In some embodiments, a first transcription factor may work with a second transcription factor. The transcription factor may be putative. The transcription factor may be selected or identified as a neuronal-specific transcription factor. A neuronal-specific transcription factor may be referred to as a neurogenic factor.

The cell type-specific transcription factor may be activating or repressing. For example, an activating or positive neuronal-specific transcription factor increases the differentiation of a cell into a neuron or increases expression of genes in neurons. Increased expression of a positive neuronal-specific transcription factor may improve or increase differentiation of a cell into a neuron or increase expression of genes in neurons. A repressing or negative neuronal-specific transcription factor inhibits the differentiation of a cell into a neuron or inhibits expression of genes in neurons. Knockdown or inhibition of expression of a negative neuronal-specific transcription factor may improve or increase differentiation of a cell into a neuron or increase expression of genes in neurons. Modulation of expression or protein levels of the neuronal-specific transcription factor may directly convert a stem cell to a neuron without a pluripotent stage.

Provided herein is a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2. Further provided is a polynucleotide encoding the first neuronal-specific transcription factor. In some embodiments, the first neuronal-specific transcription factor is selected from NGN3 and ASCL1, or a combination thereof.

In some embodiments, also provided herein is a second neuronal-specific transcription factor or a polynucleotide encoding the second neuronal-specific transcription factor. A first neuronal-specific transcription factor may be combined with a second neuronal-specific transcription factor. In such embodiments, the first neuronal-specific transcription factor may be selected from NGN3 and ASCL1, or a combination thereof. The second neuronal-specific transcription factor may be selected from (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, PLAGL2 (selected from “Positive Single Factor CRa-TF” in TABLE 1); (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3 (selected from “Positive sgNGN3 + CRa-TF” in TABLE 1); (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCU GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7 (selected from “Positive sgASCL1 + CRa-TF” in TABLE 1); (iv) ZIC2, SPI1, GRHL2, TFAP2C, KLFS, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3 (selected from “Negative Single Factor CRa-TF” in TABLE 2); (v) HES2, SREBF1, CIC, WHSC1, VDR, HES1, ID2, TCF21, SNAII, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HEST, ZBED4, SALL4, GLIS3, TBX22, ZNF331, EGR4, Z105, ZNF710, ZNF697, ZFP36L2, ELMSAN1, ZNF296, ZNF318, ZNF570, ZNF683, ZFP36L1, HES4, ZNF777, HESS, ZIM2, ZNF579, BMP2, CRAMP1 L, TOX3, FEZF2, HES3, ZNF791 (selected from “Negative sgNGN3+ CRa-TF” in TABLE 2); and (vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMPI L, ZNF821, KMT2A, HES3, BSX (selected from “Negative sgASCL1 CRa-TF” in TABLE 2).

In some embodiments, the second neuronal-specific transcription factor is selected from NEUROG3, SOX4, and SOX9. In some embodiments, the second neuronal-specific transcription factor is selected from LHX8, LHX6, E2F7, RUNX3, FOXH1, SOX2, HMX2, NKX2-2, HES3, and ZFP36L1. In some embodiments, the second neuronal-specific transcription factor is an activating transcription factor selected from LHX8, LHX6, E2F7, RUNX3, FOXH1, SOX2, HMX2, NKX2-2. In some embodiments, the second neuronal-specific transcription factor is a repressing transcription factor selected from HES3 and ZFP36L1.

Further provided herein is a muscle-specific transcription factor. The muscle-specific transcription factor may be selected from TWIST1, PAX3, MYOD, MYOG, SOX9, SOX10, and DMRT1. Further provided is a polynucleotide encoding the muscle-specific transcription factor.

### 3. CRISPR/CAS-BASED GENE EDITING SYSTEM

The system may be a CRISPR/Cas-based gene editing system. The CRISPR/Cas-based gene editing system can include a nuclease-inactive Cas protein (dCas) or a dCas fusion protein to target regions in a TF gene, or a promoter or regulatory element of the TF gene or a portion thereof, causing activation or repression of endogenous expression of the TF. The system may be a CRISPR/Cas9-based gene editing system. “Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. A Cas protein, such as a Cas9 protein, forms a complex with the 3′ end of the sgRNA (also referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, such as Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a shod sequence recognized by the Cas9 nuclease that is required for DNA cleavage. Different Type II systems have differing PAM requirements. The Streptococcus pyogenes CRISPR system may have the PAM sequence for this Cas9 (SpCas9) as 5′-NRG-3′, where R is either A or G, and characterized the specificity of this system in human cells. A unique capability of the CRISPR/Cas9-based gene editing system is the straightforward ability to simultaneously target multiple distinct genomic loci by co-expressing a single Cas9 protein with two or more sgRNAs. For example, the S. pyogenes Type II system naturally prefers to use an “NGG” sequence, where “N” can be any nucleotide, but also accepts other PAM sequences, such as “NAG” in engineered systems (Hsu et al., Nature Biotechnology 2013 doi:10.1038/nbt.2647). Similarly, the Cas9 derived from Neisseria meningitidis (NmCas9) normally has a native PAM of NNNNGATT (SEQ ID NO: 12), but has activity across a variety of PAMs, including a highly degenerate NNNNGNNN PAM (SEQ ID NO: 13) (Esvelt et al. Nature Methods 2013 doi:10.1038/nmeth.2681).

A Cas9 molecule of S. aureus recognizes the sequence motif NNGRR (R=A or G) (SEQ ID NO: 8) and directs cleavage of a target nucleic acid sequence 1 to 10, e.g., 3 to 5, by upstream from that sequence. In certain embodiments, a Cas9 molecule of S. aureus recognizes the sequence motif NNGRRN (R=A or G) (SEQ ID NO: 9) and directs cleavage of a target nucleic acid sequence 1 to 10, e.g., 3 to 5, bp upstream from that sequence. In certain embodiments, a Cas9 molecule of S. aureus recognizes the sequence motif NNGRRT (R=A or G) (SEQ ID NO: 10) and directs cleavage of a target nucleic acid sequence 1 to 10, e.g., 3 to 5, bp upstream from that sequence. In certain embodiments, a Cas9 molecule of S. aureus recognizes the sequence motif NNGRRV (R=A or G) (SEQ ID NO: 11) and directs cleavage of a target nucleic acid sequence 1 to 10, e.g., 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in genome editing and treating genetic diseases, The CRISPR/Cas9-based engineered systems can be designed to target any gene, including genes involved in a genetic disease, aging, tissue regeneration, or wound healing, The CRISPR/Cas9-based gene editing systems can include a Cas9 protein or Cas9 fusion protein and at least one gRNA. In certain embodiments, the system comprises two gRNA molecules. The Cas9 fusion protein may, for example, include a domain that has a different activity that what is endogenous to Cas9, such as a transactivation domain.

The target gene can be involved in differentiation of a cell or any other process in which activation of a gene can be desired, or can have a mutation such as a frameshift mutation or a nonsense mutation. In some embodiments, the target or target gene includes a gene, or portion thereof, for a putative transcription factor. The CRISPR/Cas9-based gene editing system may or may not mediate off-target changes to protein-coding regions of the genome. The CRISPR/Cas9-based gene editing system may bind and recognize a target region.

a. Cas Protein

The CRISPR/Cas9-based gene editing system can include a Cas9 protein or a Cas fusion protein, In some embodiments, the Cas protein is a Cas12 protein (also referred to as Cpf1), such as a Cas12a protein. The Cas12 protein can be from any bacterial or archaea species, including, but not limited to, Francisella novicida, Acidaminococcus sp., Lachnospiraceae sp., and Prevotella sp. In some embodiments, the Cas protein is a Cas9 protein. Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacterjejuni, Campylobacter lari, Candidatus Puniceispirillum, Clostridium cellulolyticum, Clostridium perfringens, Corynebactenurn accolens, Corynebacterium diphtheria, Corynebacterium matruchotii, Dinoroseobacter shibae, Eubactedurn dolichum, gamma proteobacteriurn, Gluconacetobacter diazotrophicus, Haemophilus parainfluenzae, Haemophilus sputorum, Helicobacter canadensis, Helicobacter cinaedi, Helicobacter mustelae, ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatulens, Ralstonia syzygii, Rhodopseudomonas palustris, Rhodovulum sp,, Simonsiella muelleeri, Sphingomonas sp., Spomlactobacillus vineae, Staphylococcus lugdunensis, Streptococcus sp., Subdoligranulum sp., Tistrella mobillis Treponema sp., or Verminephrobacter eiseniae. In certain embodiments, the Cas9 molecule is a Streptococcus pyogenes Cas9 molecule (also referred herein as “SpCas9”). In certain embodiments, the Cas9 molecule is a Staphylococcus aureus Cas9 molecule (also referred herein as “SaCas9”).

A Cas molecule or a Cas fusion protein can interact with one or more gRNA molecule and, in concert with the gRNA molecule(s), can localize to a site which comprises a target domain, and in certain embodiments, a PAM sequence. The ability of a Cas molecule or a Cas fusion protein to recognize a PAM sequence can be determined, e.g., using a transformation assay as known in the art.

In certain embodiments, the ability of a Cas molecule or a Cas fusion protein to interact with and cleave a target nucleic acid is protospacer-adjacent motif (PAM) sequence dependent. A PAM sequence is a sequence in the target nucleic acid. In certain embodiments, cleavage of the target nucleic acid occurs upstream from the PAM sequence. Cas molecules from different bacterial species can recognize different sequence motifs (e.g., PAM sequences). In certain embodiments, a Cas12 molecule of Francisella novicida recognizes the sequence motif TTTN (SEQ ID NO: 35). In certain embodiments, a Cas9 molecule of S. pyogenes recognizes the sequence motif NGG (SEQ ID NO: 1) and directs cleavage of a target nucleic acid sequence 1 to 10, e.g., 3 to 5, bp upstream from that sequence. In certain embodiments, a Cas9 molecule of S. thermophilus recognizes the sequence motif NGGNG (SEQ ID NO: 5) and/or NNAGAAW (W=A or T) (SEQ ID NO: 6) and directs cleavage of a target nucleic acid sequence 1 to 10, e.g., 3 to 5, by upstream from these sequences. In certain embodiments, a Cas9 molecule of S. mutans recognizes the sequence motif NGG (SEQ ID NO: 1) and/or NAAR (R=A or G) (SEQ ID NO: 7) and directs cleavage of a target nucleic acid sequence 1 to 10, e.g., 3 to 5 bp, upstream from this sequence. In certain embodiments, a Cas9 molecule of S. aureus recognizes the sequence motif NNGRR (R=A or G) (SEQ ID NO: 8) and directs cleavage of a target nucleic acid sequence 1 to 10, e.g., 3 to 5, bp upstream from that sequence. In certain embodiments, a Cas9 molecule of S. aureus recognizes the sequence motif NNGRRN (R=A or G) (SEQ ID NO: 9) and directs cleavage of a target nucleic acid sequence 1 to 10, e.g., 3 to 5, by upstream from that sequence, In certain embodiments, a Cas9 molecule of S. aureus recognizes the sequence motif NNGRRT (R=A or G) (SEQ ID NO: 10) and directs cleavage of a target nucleic acid sequence 1 to 10, e.g., 3 to 5, bp upstream from that sequence. In certain embodiments, a Cas9 molecule of S. aureus recognizes the sequence motif NNGRRV (R=A or G; V=A or C or G) (SEQ ID NO: 11) and directs cleavage of a target nucleic acid sequence 1 to 10, e.g., 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

In certain embodiments, the vector encodes at least one Cas9 molecule that recognizes a Protospacer Adjacent Motif (PAM) of either NNGRRT (SEQ ID NO: 10) or NNGRRV (SEQ ID NO: 11). In certain embodiments, the at least one Cas9 molecule is an S. aureus Cas9 molecule. In certain embodiments, the at least one Cas9 molecule is a mutant S. aureus Cas9 molecule.

The Cas protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence include; D10A, E762A, H840A, N854A, N863A, and/or D986A. Exemplary mutations with reference to the S. aureus Cas9 sequence include D10A and N580A. In certain embodiments, the Cas9 molecule is a mutant S. aureus Cas9 molecule. In some embodiments, the dCas9 is a Cas9 molecule that includes at least two mutations selected from D10A, E762A, H840A, N854A, N863A, and/or D986A, with reference to the S. pyogenes Cas9 sequence. In some embodiments, the Cas protein is a dCas9 protein. In some embodiments, the Cas protein is a dCas12 protein.

In certain embodiments, the mutant S. aureus Cas9 molecule comprises a D10A mutation. The nucleotide sequence encoding this mutant S. aureus Cas9 is set forth in SEQ ID NO: 22.

In certain embodiments, the mutant S. aureus Cas9 molecule comprises a N580A mutation. The nucleotide sequence encoding this mutant S. aureus Cas9 molecule is set forth in SEQ ID NO: 23.

A polynucleotide encoding a Cas9 molecule can be a synthetic polynucleotide. For example, the synthetic polynucleotide can be chemically modified. The synthetic polynucleotide can be codon optimized, e.g., at least one non-common codon or less-common codon has been replaced by a common codon. For example, the synthetic polynucleotide can direct the synthesis of an optimized messenger mRNA, e.g., optimized for expression in a mammalian expression system, e.g., described herein.

Additionally or alternatively, a nucleic acid encoding a Cas9 molecule or Cas9 polypeptide may comprise a nuclear localization sequence (NLS). Nuclear localization sequences are known in the art. An exemplary codon optimized nucleic acid sequence encoding a Cas9 molecule of S. pyogenes is set forth in SEQ ID NO: 14. The corresponding amino acid sequence of an S. pyogenes Cas9 molecule is set forth in SEQ ID NO: 15.

Exemplary codon optimized nucleic acid sequences encoding a Cas9 molecule of S. aureus, and optionally containing nuclear localization sequences (NLSs), are set forth in SEQ ID NOs: 16-20 and 24-25. Another exemplary codon optimized nucleic acid sequence encoding a Cas9 molecule of S. aureus comprises the nucleotides 1293-4451 of SEQ ID NO: 27. An amino acid sequence of an S. aureus Cas9 molecule is set forth in SEQ ID NO: 21. An amino acid sequence of an S. aureus Cas9 molecule is set forth in SEQ ID NO: 26.

b. Fusion Protein

Alternatively or additionally, the CRISPR/Cas-based gene editing system can include a fusion protein. The fusion protein can comprise two heterologous polypeptide domains, wherein the first polypeptide domain comprises a DNA binding protein such as a Cas protein, a zinc finger protein, or a TALE protein, and the second polypeptide domain has an activity such as transcription activation activity, transcription repression activity, transcription release factor activity, histone modification activity, nuclease activity, nucleic acid association activity, methylase activity, or demethylase activity. The fusion protein can include a first polypeptide domain such as a Cas9 protein or a mutated Cas9 protein, fused to a second polypeptide domain that has an activity such as transcription activation activity, transcription repression activity, transcription release factor activity, histone modification activity, nuclease activity, nucleic acid association activity, methylase activity, or demethylase activity. In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

i) Transcription Activation Activity

The second polypeptide domain can have transcription activation activity, i.e., a transactivation domain. For example, gene expression of endogenous mammalian genes, such as human genes, can be achieved by targeting a fusion protein of a first polypeptide domain, such as dCas9 or dCas12 and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, or p300. For example, the fusion protein may be dCas9-VP64. In other embodiments, the Cas9 protein may be VP64-dCas9-VP64 (SEQ ID NO: 36, encoded by polynucleotide of SEQ ID NO: 37). In other embodiments, the fusion protein that activates transcription may be dCas9-p300. In some embodiments, p300 may comprise a polypeptide of SEQ ID NO: 159 or SEQ ID NO:160.

ii) Transcription Repression Activity

The second polypeptide domain can have transcription repression activity. The second polypeptide domain can have a Kruppel associated box activity, such as a KRAB domain, ERF repressor domain activity, Mxil repressor domain activity, SID4X repressor domain activity, Mad-SID repressor domain activity, or TATA box binding protein activity. For example, the fusion protein may be dCas9-KRAB.

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 may comprise a polypeptide of SEQ ID NO: 159 or SEQ ID NO: 160.

v) Nuclease Activity

The second polypeptide domain can have nuclease activity that is different from the nuclease activity of the Cas9 protein. A nuclease, ora protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease,

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-tun-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

vii) Methylase Activity

The second polypeptide domain can have rnethylase activity, which involves transferring a methyl group to DNA, RNA, protein, small molecule, cytosine or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1.

c. gRNA

The CRISPR/Cas-based gene editing system includes at least one gRNA molecule. For example, the CRISPR/Cas-based gene editing system may include two gRNA molecules. The gRNA provides the targeting of a CRISPR/Cas-based gene editing system. The gRNA is a fusion of two noncoding RNAs: a crRNA and a tracrRNA. In some embodiments, the polynucleotide includes a crRNA and/or a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The “target region”, “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPRICas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The scaffold may comprise a polynucleotide sequence of SEQ ID NO: 158. The CRISPR/Cas9-based gene editing system may include at least one gRNA, wherein the gRNAs target different DNA sequences. The target DNA sequences may be overlapping. The target sequence or protospacer is followed by a PAM sequence at the 3′ end of the protospacer in the genome. Different Type II systems have differing PAM requirements. For example, the Streptococcus pyogenes Type II system uses an “NGG” sequence (SEQ ID NO: 1), where “N” can be any nucleotide. In some embodiments, the PAM sequence may be “NGG”, where “N” can be any nucleotide. In some embodiments, the PAM sequence may be NNGRRT (SEQ ID NO: 10) or NNGRRV (SEQ ID NO: 11). The at least one gRNA molecule can bind and recognize a target region.

The number of gRNA molecule encoded by a genetic construct (e.g., an AAV vector) can be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNAs encoded by a presently disclosed vector can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (e.g., an AAV vector) encodes one gRNA molecule, i.e., a first gRNA molecule, and optionally a Cas9 molecule. In certain embodiments, a first genetic construct (e.g., a first AAV vector) encodes one gRNA molecule, i.e,, a first gRNA molecule, and optionally a Cas9 molecule, and a second genetic construct (e.g., a second AAV vector) encodes one gRNA molecule, i.e., a second gRNA molecule, and optionally a Cas9 molecule.

The gRNA molecule comprises a targeting domain, which is a polynucleotide sequence complementary to the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region within or near a gene encoding a transcription factor. In certain embodiments, the gRNA can target at least one of exons, introns, the promoter region, the enhancer region, or the transcribed region of the gene.

In some embodiments, the gRNA targets a neuronal-specific transcription factor. The gRNA may include a targeting domain that comprises a polynucleotide sequence corresponding to at least one of SEQ ID NOs: 38-97, as shown in TABLE 3, or a complement thereof or a variant thereof. The gRNA may target a polynucleotide comprising a sequence selected from SEQ ID NOs: 38-97, or a complement, a portion, or a variant thereof. The gRNA may be encoded by a polynucleotide comprising a sequence selected from SEQ ID NOs: 38-97, or a complement, a portion, or a variant thereof. The gRNA may comprise a polynucleotide sequence corresponding to (for example, a RNA version thereof) at least one of SEQ ID NOs: 38-97, or a complement, a portion, or a variant thereof.

TABLE 3
Exemplary gRNAs targeting putative neuronal-
specific transcription factors.

| Gene | sgRNA Sequence | SEQ ID NO |
|---|---|---|
| Scrambled 1 | TGTCGTGATGCGTAGACGG | 38 |
| Scrambled 2 | TCATCAAGGAGCATTCCGT | 39 |
| NEUROG3 | CTCGAGAGAGCAAACAGAG | 40 |
| RFX4 | ATAGAAGGGGGAAGTCGGA | 41 |
| SOX4 | CATGCCAAACCCCTCCCCC | 42 |
| NEUROD1 | TGAGGGGAGCGGTTGTCGG | 43 |
| INSM1 | CGCCGGGCGGGGCGACCAG | 44 |
| KLF7 | AGCGCGAGCGCAAGGGACA | 45 |
| SOX9 | CTGGGTGACGAGGCGGGAG | 46 |
| SOX17 | CAAGGCTACACCTGCCCCC | 47 |
| NEUROG1 | TAGCCCGAGCCGACTCCCG | 48 |
| SP8 | GCGCGCGCCGTGAGGTCAT | 49 |
| KLF4 | CTCCCTTCCATCGTTGCTA | 50 |
| SMAD1 | CCGGGCCGGGAATTTGGAG | 51 |
| OVOL1 | CGACAGGTAACAAATAGGT | 52 |
| NR5A1 | AATACCCCTATCTATCTGG | 53 |
| ATOH1 | GCCTGCCCGCGCCCTCCAT | 54 |
| NEUROG2-1 | GCAGCGAGGACGAAGGCGG | 55 |
| NEUROG2-2 | GGAAAGGCGGTGAAGAAAG | 56 |
| SOX18 | GCCTCAGCGGAATCCCGCC | 57 |
| ASCL1 | GAGGAGGAGGGGGAGTTTA | 58 |
| ASCL1 | AATGGAGAGTTTGCAAGGAG | 59 |
| (sublibrary) | | |
| ATOH7 | ACTAACACACCATCTGGAG | 60 |
| ATOH8 | CGGGGCGGTTGTGCAGGAG | 61 |
| ATOH1-2 | GGCTGAGAAGACACGCGAC | 62 |
| ATOH1-3 | CACTCGGAGATCACACACC | 63 |
| ATOH1-4 | CACGCGACCGGCGCGAGGA | 64 |
| ATOH1-5 | TGCGGAGCCGGCTCTCGGC | 65 |
| NR5A1-2 | AGAGAAACACCAACAAAGA | 66 |
| NR5A1-3 | GGCCTGCAGAGTCACGTGG | 67 |
| NR5A1-4 | TGCCCCCACGTGACTCTGC | 68 |
| NR5A1-5 | GGGCCACCGGAGGCCCAAT | 69 |
| LHX6 | AGGAGGAGGACTACCMGA | 70 |
| LHX8 | CGGGGAACACCGGGCTAAA | 71 |
| E2F7 | GCGCCAAGACTCCGAGGGG | 72 |
| RUNX3 | CCTGCCGGAGGCCGCCCAA | 73 |
| FOXH1 | CCACCCAAAGGCAACTCAG | 74 |
| SOX2 | GGATACAAAGGTTTCTCAG | 75 |
| HMX2 | AGGCCCTCGGCGCGCTCTG | 76 |
| NKX22 | CCCTCTAGAGCAAGATGAG | 77 |
| ELMSAN1 | GGCGTCCTTAAACCTCAGG | 78 |
| GCM2 | ACAGTCCCAGGAACGGAGG | 79 |
| HES1 | GTGGACCGCGCCCCCCCAT | 80 |
| HES7 | CCCTCTAGGACCCGGCACG | 81 |
| TOX3 | AGAAGAGGGGCCCCGGAGA | 82 |
| DMRT | GGACCCTGCAGCAAAGCCC | 83 |
| BMP2 | CCGCCCGCTCGGGGATCCC | 84 |
| ZFP36L1 | CTTCCCTACCCGGCGCTTC | 85 |
| ERF | GAGCGTGTGTGTGAGTGCGC | 86 |
| PRDM1 | CGGCTGTGCTAGCAATCTGG | 87 |
| OL1G3 | GAGCCCTCCTATCTATCCT | 88 |
| HIC1 | GCTGTGCGCCGTGCCCGCCC | 89 |
| SOX3 | CGGAGGACCCGTGATTGAC | 90 |
| FOXJ1 | GCTCGGCTCATTCCCGCCCG | 91 |
| SOX10 | CCCTGAGTGTTGGGGATGA | 92 |
| KLF6 | TCCCGTGGCTCCCGGCCCGG | 93 |
| PLAGL2 | GCCCCGGCCGCTCTAGCCCG | 94 |
| S. aureus | TCATCAAGGAGCATTCCGT | 95 |
| Scrambled | | |
| S. aureus | ATGACAACAAGAACCCCGGA | 96 |
| ZFP36L1 | | |
| S. aureus | CCCTTCCCCGGGAGGTGTGG | 97 |
| HES3 | | |

In some embodiments, the gRNA targets a muscle-specific transcription factor. The muscle-specific transcription factor may be selected from TWIST1, PAX3, MYOD, MYOG, SOX9, SOX10, and DMRT1. The gRNA may include a targeting domain that comprises a polynucleotide sequence corresponding to at least one of SEQ ID NOs: 98-104, as shown in TABLE 5, or a complement thereof or a variant thereof. The gRNA may target a polynucleotide comprising a sequence selected from SEQ ID NOs: 98-104, or a complement, a portion, or a variant thereof. The gRNA may be encoded by a polynucleotide comprising a sequence selected from SEQ ID NOs: 98-104, or a complement, a portion, or a variant thereof. The gRNA may comprise a polynucleotide sequence corresponding to (for example, a RNA version thereof) at least one of SEQ ID NOs: 98-104, or a complement, a portion, or a variant thereof.

TABLE 5
Exemplary gRNAs targeting muscle-specific
transcription factors.

| Gene | gRNA Target Sequence | SEQ ID NO |
|---|---|---|
| TWIST1 | CGGCTAGGAGGCGGGTGGA | 98 |
| PAX3 | CGGGCCAACCTTCTCTCCT | 99 |
| MYOD | CGCGCACGCCAGTGTGGAG | 100 |
| MYOG | GGGCCATGCGGGAGAAAGA | 101 |
| SOX9 | GGAGGGGATCGCAGCCAAA | 102 |
| SOX10 | GGAGGAGCCCTGAGTGTTG | 103 |
| DMRT1 | GCAAGCAGCTGGAGAGCGG | 104 |

A cell transformed or transcribed with the system as detailed herein may express at least one gRNA. The cells may each independently include one gRNA and target one putative transcription factor. The level of the at least one gRNA in a cell may be determined by any suitable means known in the art, such as, for example, deep sequencing. At least one gRNA may be enriched in a cell. For example, at least one gRNA may be enriched in a cell, the cell having high expression of a reporter protein. “Enriched” may refer to a statistically significant (p<0.05) increase in gRNA abundance in cells with high reporter gene expression. This may be calculated using the differential expression analysis package DESeq2 in R. The gRNA, or at least one gRNA in a cell, may increase the expression of the reporter protein in the cell by about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, or about 90% relative to a control. A control may be cell with a non-targeting gRNA. In some embodiments, the gRNA increases the expression of the reporter protein in the cell by about 2-50% relative to a non-targeting gRNA.

d. Genetic Constructs

The system for identifying a cell type-specific transcription factor, or for increasing expression of a cell type-specific gene, or one or more components thereof, may be encoded by or comprised within a genetic construct. Genetic constructs may include polynucleotides such as vectors and plasmids. The construct may be recombinant. In some embodiments, the genetic construct comprises a promoter that is operably linked to the polynucleotide encoding at least one gRNA molecule and/or a Cas molecule or fusion protein. In some embodiments, the genetic construct comprises a promoter that is operably linked to the polynucleotide encoding at least one gRNA molecule and/or a dCas molecule or fusion protein. In some embodiments, the genetic construct comprises a promoter that is operably linked to the polynucleotide encoding at least one gRNA molecule and/or a Cas9 molecule or fusion protein. In some embodiments, the promoter is operably linked to the polynucleotide encoding a gRNA molecule, reporter protein, neuronal marker, and/or a Cas9 molecule. In some embodiments, the promoter is operably linked to the polynucleotide encoding a first gRNA molecule, a second gRNA molecule, reporter protein, neuronal marker, and/or a Cas9 molecule. The genetic construct may be present in the cell as a functioning extrachromosomal molecule. The genetic construct may be a linear minichromosome including centromere, telomeres, or plasmids or cosmids. The genetic construct may be transformed or transduced into a cell. The genetic construct may be formulated into any suitable type of delivery vehicle including, for example, a viral vector, lentiviral expression, mRNA electroporation, and lipid-mediated transfection. Further provided herein is a cell transformed or transduced with a system or component thereof' as detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

Further provided herein is a viral delivery system. Viral delivery systems may include, for example, lentivirus, retrovirus, mRNA electroporation, or nanoparticles. In some embodiments, the vector is an adeno-associated virus (AAV) vector. The AAV vector is a small virus belonging to the genus Dependovirus of the Parvoviridae family that infects humans and some other primate species. AAV vectors may be used to deliver CRISPRICas9-based gene editing systems using various construct configurations. For example, AAV vectors may deliver Cas9 and gRNA expression cassettes on separate vectors or on the same vector. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2i6, AAV2/7, AAV218, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 4. SYSTEM FOR INCREASING NEURONAL-SPECIFIC TRANSCRIPTION OF A GENE

Provided herein is a system for increasing neuronal-specific transcription of a gene, or for increasing expression of a neuronal-specific gene. The system may include a first gRNA targeting a first neuronal-specific transcription factor, regulatory region, promoter region, or portion thereof; and a Cas protein or a fusion protein, as detailed above. The system may include a first gRNA targeting a first neuronal-specific transcription factor, regulatory region, promoter region, or portion thereof; a second gRNA targeting a second neuronal-specific transcription factor, regulatory region, promoter region, or portion thereof; and a Cas protein ora fusion protein, as detailed above. In some embodiments, the second neuronal-specific transcription factor is a positive or activating transcription factor, and the second polypeptide domain of the fusion protein has transcription activation activity. In some embodiments, the second neuronal-specific transcription factor is a negative or repressing transcription factor, and the second polypeptide domain of the fusion protein has transcription repression activity.

### 5. SYSTEM FOR IDENTIFYING A CELL TYPE-SPECIFIC TRANSCRIPTION FACTOR

Provided herein are compositions and methods for selecting or identifying a cell type-specific transcription factor, such as, for example, a neuronal-specific transcription factor or a muscle-specific transcription factor or a chondrocyte-specific transcription factor. The system includes a polynucleotide encoding a reporter protein and a cell type marker; a Cas protein or fusion protein as detailed above; and a library of gRNAs that targets putative transcription factors. Further provided herein is a cell type-specific transcription factor, or a polynucleotide sequence encoding the cell type-specific transcription factor, or a polynucleotide sequence encoding a gRNA targeting the cell type-specific transcription factor, as selected or identified by the compositions and methods detailed herein.

a. Reporter Protein

The polynucleotide may encode a reporter protein. A reporter protein is encoded by a reporter gene and causes some determinable or detectable characteristic in a recombinant system simultaneously with the expression of another gene to indicate the expression of that other gene. The reporter protein is capable of generating a detectable signal. A variety of reporter proteins can be used, differing in the physical nature of signal transduction (e.g., fluorescence, electrochemical, nuclear magnetic resonance (NMR), and electron paramagnetic resonance (EPR)) and in the chemical nature of the reporter protein. In some embodiments, the signal from the reporter protein is a fluorescent signal.

In some embodiments, the reporter protein is a fluorescent protein. Fluorescent proteins include, for example, luciferase, enhanced blue fluorescent protein (EBFP), enhanced blue fluorescent protein-2 (EBFP2), mKATE, iRFP (infrared fluorescent protein), enhanced yellow fluorescent protein (EYFP), yellow fluorescent protein (YFP), Katushka, Ds-Red express, red fluorescent protein, red fluorescent protein turbo, TurboRFP, TagRFP, green fluorescent protein (GFP), blue fluorescent protein (BFP), cyan fluorescent protein(CFP), enhanced green fluorescent protein (EGFP), AcGFP, TurboGFP, Emerald, Azami Green, ZsGreen, Sapphire, T-Sapphire, enhanced cyan fluorescent protein (ECFP), mCFP, Cerulean, CyPet, AmCyanl, Midori-Ishi Cyan, mTFPI (Teal), Topaz, Venus, mCitrine, YPet, PhiYFP, ZsYellowI, mBanana, Kusabira Orange, mOrange, dTomato, dTomato-Tandem, DsRed, DsRed2, DsRed-Express (TI), DsRed-Monomer, mTangerine, mStrawberry, AsRed2, rnRFPI, JRed, rnCherry, HcRedI, mRaspberry, HcRedI, HcRed-Tandem, mPlum, and AQ143, or a combination thereof. In some embodiments, the reporter protein comprises mCherry. mCherry may comprise a polypeptide having an amino acid sequence of SEQ ID NO: 28 and may be encoded by a polynucleotide comprising SEQ ID NO: 29, In some embodiments, the reporter protein is any polypeptide that may be identified by irnmunohistochemistry or antibody staining

A cell transfected or transformed with the polynucleotide may express the reporter protein. The level of expression of the reporter protein, in a cell for example, may be determined. The level of expression of the reporter protein may be determined at various time points after transfection of the cell with the system detailed herein. For example, the level of expression of the reporter protein in a cell maybe determined after about 1, 2, 3, 4, 5, 6, 7, 8, 9, or 10 days from transduction. In some embodiments, the level of expression of the reporter protein in a cell is determined after about 4 days from transduction. Fluorescent proteins can be assayed by any suitable means known in the art, for example, by FACS or flow cytometry or fluorescence microscopy. In some embodiments, a cell transfected or transformed with the polynucleotide has a high expression of the reporter protein relative to a control. The control may be another cell or cells transfected or transformed with a polynucleotide including a different gRNA. “High expression” of the reporter protein may be defined as being in the top 5% expression levels among the population of cells.

b. Cell Type Marker

The polynucleotide may encode a marker indicating expression in a certain cell type or state or stage. For example, the polynucleotide may encode a neuronal marker. A neuronal marker is a gene that is expressed only in or predominantly in neuronal cells. The neuronal marker may be a subtype-specific marker that is only expressed in certain subtypes of neurons. The neuronal marker may be a pan-neuronal marker. A pan-neuronal marker is a gene that is expressed only in or predominantly in neuronal cells and in most of the neuronal cells. The pan-neuronal marker may also be referred to as a neuronal lineage marker. The neuronal marker may be expressed at any point in neurogenesis and in cells that have differentiated into a neuron. Neuronal markers may be selected from, for example, TUBB3, NEUROD1, NEUROG1, NEUROG2, ASCU, SYN1, NCAM, and MAP2. In some embodiments, the pan-neuronal marker is TUBB3. TUBB3 is a gene that encodes the polypeptide beta-3-tubulin (also referred to as beta-tubulin III), which is a microtubule element of the tubulin family found almost exclusively in neurons. In some embodiments, the cell-type specific transcription factor is a neuronal-specific transcription factor, the cell type marker is a neuronal marker, and the neuronal marker comprises TUBB3.

In other embodiments, the cell type marker is a muscle or myogenic marker. A muscle or rnyogenic marker is a gene that is expressed only in or predominantly in muscle cells. The muscle or myogenic marker may be a subtype-specific marker that is only expressed in certain subtypes of muscle cells. The muscle or rnyogenic marker may be a pan-muscle or pan-myogenic marker. A pan-muscle or pan-myogenic marker is a gene that is expressed only in or predominantly in muscle cells and in most of the muscle cells. The myogenic marker may comprise PAX7. In some embodiments, the cell-type specific transcription factor is a muscle-specific transcription factor, the cell type marker is a myogenic marker, and the myogenic marker comprises PAX7.

In other embodiments, the cell type marker is a collagen marker. A collagen marker is a gene that is expressed only in or predominantly in chondrocytes. The collagen marker may be a subtype-specific marker that is only expressed in certain subtypes of chondrocytes. The collagen marker may be a pan-collagen marker. A pan-collagen marker is a gene that is expressed only in or predominantly in chondrocytes and in most of the chondrocytes. The collagen marker may comprise COL2A1. In some embodiments, the cell-type specific transcription factor is a chondrocyte-specific transcription factor, the cell type marker is a collagen marker, and the collagen marker comprises COL2A1.

The polynucleotide encoding the reporter protein may be operably linked to a polynucleotide encoding a cell type marker, as detailed below. The polynucleotide encoding the reporter protein may be in the same reading frame as the polynucleotide encoding the cell type marker. As such, the reporter protein may serve as an expression or translational reporter of the cell type marker.

A cell transfected or transformed with the polynucleotide may express the cell type marker. The level of expression of the cell type marker, in a cell for example, may be determined. The level of expression of the cell type marker may be determined at various time points after transfection of the cell with the system detailed herein. For example, the level of expression of the cell type marker in a cell maybe determined after about 1, 2, 3, 4, 5, 6, 7, 8, 9, or 10 days from transduction. Cell type markers can be assayed by any suitable means known in the art, for example, by immunohistochemistry, qRT-PCR, and RNA sequencing.

c. Library of gRNAs

The system for selecting or identifying a transcription factor may further include a library of gRNAs. The library of gRNAs may target putative transcription factors. For example, a gRNA may target the promoter of a gene encoding a transcription factor. Each gRNA may be different. The library of gRNAs may include a plurality of gRNAs, each gRNA targeting a putative transcription factor. In some embodiments, each gRNA targets a different putative transcription factor. Some gRNAs may target the same putative transcription factor, with each gRNA targeting a different portion of the gene encoding the transcription factor. In some embodiments, the different portions may overlap. In some embodiments, the gRNA library may include 1, 2, 3, 4, 5, 6, 7, 8, 9, or 10 gRNAs for each transcription start site of a transcription factor. The gRNA library may include at least about 1000, at least about 2000, at least about 3000, at least about 4000, at least about 5000, at least about 6000, at least about 7000, at least about 8000, or at least about 9000 gRNAs.

### 6. PHARMACEUTICAL COMPOSITIONS

Further provided herein are pharmaceutical compositions comprising the above-described genetic constructs or systems. The systems, or at least one component thereof, as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizer's include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powder's, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent may be a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/mL. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. W09324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

### 7. ADMINISTRATION

The systems, or at least one component thereof, as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. For veterinary use, the DNA targeting systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound.

The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system.

### 8. METHODS

a. Methods of increasing Neuronal Maturation of a Stem Cell

Provided herein are methods of increasing neuronal maturation of a stem cell, or methods of increasing maturation of a stern cell-derived neuron. The method may include (a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; or (b) increasing in the stem cell the level of a first neuronal-specific transcription factor selected from NGN3 and ASCL1, or a combination thereof, and increasing in the stem cell the level of a second neuronal-specific transcription factor, wherein the second neuronal-specific transcription factor is an activating or positive neuronal-specific transcription factor, In other embodiments, the method may include increasing in the stem cell the level of a first neuronal-specific transcription factor selected from NGN3 and ASCL1, or a combination thereof; and decreasing in the stem cell the level of a second neuronal-specific transcription factor, wherein the second neuronal-specific transcription factor is a repressing or negative neuronal-specific transcription factor.

In some embodiments, increasing the level of the first neuronal-specific transcription factor comprises at least one of: (a) administering to a stern cell a polynucleotide encoding the first neuronal-specific transcription factor; (b) administering to a stem cell a polypeptide comprising the first neuronal-specific transcription factor; and (c) administering to a stem cell a gRNA targeting the first neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments, increasing the level of the second neuronal-specific transcription factor comprises at least one of: (a) administering to a stern cell a polynucleotide encoding the second neuronal-specific transcription factor; (b) administering to a stem cell a polypeptide comprising the second neuronal-specific transcription factor; and (c) administering to a stem cell a gRNA targeting the second neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments,decreasing the level of the second neuronal-specific transcription factor comprises administering to a stem cell a gRNA targeting the second neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription repression activity.

b. Methods of Increasing the Conversion of a Stem Cell to a Neuron

Provided herein are methods of increasing the conversion of a stem cell to a neuron. The method may include (a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SF8, OVOL1, NEUROG2, ERF, PRDM1, OLIG3, HIC1, SOX3, FOXJ1, SOX10, KLF6, ASCL1, and PLAGL2; or (b) increasing in the stem cell the level of a first neuronal-specific transcription factor selected from NGN3 and ASCL1, or a combination thereof, and increasing in the stem cell the level of a second neuronal-specific transcription factor, wherein the second neuronal-specific transcription factor is an activating or positive neuronal-specific transcription factor. In other embodiments, the method may include increasing in the stem cell the level of a first neuronal-specific transcription factor selected from NGN3 and ASCL1, or a combination thereof; and decreasing in the stem cell the level of a second neuronal-specific transcription factor, wherein the second neuronal-specific transcription factor is a repressing or negative neuronal-specific transcription factor.

In some embodiments, increasing the level of the first neuronal-specific transcription factor comprises at least one of; (a) administering to a stem cell a polynucleotide encoding the first neuronal-specific transcription factor; (b) administering to a stem cell a polypeptide comprising the first neuronal-specific transcription factor; and (c) administering to a stem cell a gRNA targeting the first neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments, increasing the level of the second neuronal-specific transcription factor comprises at least one of: (a) administering to a stem cell a polynucleotide encoding the second neuronal-specific transcription factor; (b) administering to a stem cell a polypeptide comprising the second neuronal-specific transcription factor; and (c) administering to a stem cell a gRNA targeting the second neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments,decreasing the level of the second neuronal-specific transcription factor comprises administering to a stem cell a gRNA targeting the second neuronal-specific transcription factor, regulatory region, promoter region, or portion thereof and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription repression activity.

c. Methods of Treating a Subject

Provided herein are methods of treating a subject in need thereof. The method may include (a) increasing in the stern cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SPB, OVOL1, NEUROG2, ERF, PRDM1, OLIG3, HIC1, SOX3, FOXJ1, SOX10, KLF6, ASCL1, and PLAGL2; or (b) increasing in the stem cell in the subject the level of a first neuronal-specific transcription factor selected from NGN3 and ASCL1, or a combination thereof, and increasing in the stem cell in the subject the level of a second neuronal-specific transcription factor, wherein the second neuronal-specific transcription factor is an activating or positive neuronal-specific transcription factor. In other embodiments, the method may include increasing in the stem cell in the subject the level of a first neuronal-specific transcription factor selected from NGN3 and ASCL1, or a combination thereof; and decreasing in the stem cell in the subject the level of a second neuronal-specific transcription factor, wherein the second neuronal-specific transcription factor is a repressing or negative neuronal-specific transcription factor.

In some embodiments, increasing the level of the first neuronal-specific transcription factor comprises at least one of: (a) administering to a stem cell a polynucleotide encoding the first neuronal-specific transcription factor; (b) administering to a stem cell a polypeptide comprising the first neuronal-specific transcription factor; and (c) administering to a stem cell a gRNA targeting the first neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments, increasing the level of the second neuronal-specific transcription factor comprises at least one of: (a) administering to a stem cell a polynucleotide encoding the second neuronal-specific transcription factor; (b) administering to a stern cell a polypeptide comprising the second neuronal-specific transcription factor; and (c) administering to a stern cell a gRNA targeting the second neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription activation activity.

In some embodiments,decreasing the level of the second neuronal-specific transcription factor comprises administering to a stem cell a gRNA targeting the second neuronal-specific transcription factor, regulatory region, promoter region, or portion thereof, and a fusion protein, wherein the fusion protein comprises two heterologous polypeptide domains, wherein the first polypeptide domain comprises a DNA binding protein such as a Cas protein, a zinc finger protein, or a TALE protein, and the second polypeptide domain has transcription repression activity.

d. Methods of Screening for a Neuronal-Specific Transcription Factor

Provided herein are methods of screening for a neuronal-specific transcription factor. The method may include transducing a population of cells with the system of any one of claims 1-3 at a multiplicity of infection (MOI) of about 0.2, such that a majority of the cells each independently includes one gRNA and targets one putative transcription factor; determining a level of expression of the reporter protein in each cell; determining a level of the gRNA in each cell having a high expression of the reporter protein, wherein high expression of the reporter protein is defined as being in the top 5% among the population of cells; and selecting the putative transcription factor as a neuronal-specific transcription factor when the putative transcription factor corresponds to at least two gRNAs enriched in the cell having a high expression of the reporter protein. “Enriched” may be a statistically significant (p<0,05) increase in gRNA abundance in cells with high reporter gene expression.

In some embodiments, the level of expression of the reporter protein in each cell is determined after about four days from transduction. In some embodiments, the level of expression of the reporter protein in each cell is determined by flow cytometry. In some embodiments, the level of the gRNA in each cell having a high expression of the reporter protein is determined by deep sequencing. In some embodiments, the gRNA increases the expression of the reporter protein in the cell by about 2-50% relative to a non-targeting gRNA.

e. Methods of Screening for a Pair of Neuronal-Specific Transcription Factors

Provided herein are methods of screening for a pair of neuronal-specific transcription factors. The methods may include transducing a population of cells with the system of any one of claims 1-3 at a multiplicity of infection (MOI) of about 0.2, such that a majority of the cells each independently includes two gRNAs and targets two putative transcription factors; determining a level of expression of the reporter protein in each cell; determining a level of the two gRNAs in each cell having a high expression of the reporter protein, wherein high expression of the reporter protein is defined as being in the top 5% among the population of cells; and selecting the two putative transcription factors as a pair of neuronal-specific transcription factors when the putative transcription factors correspond to at least two gRNAs enriched in the cell having a high expression of the reporter protein.

In some embodiments, the level of expression of the reporter protein in each cell is determined after about four days from transduction. In some embodiments, the level of expression of the reporter protein in each cell is determined by flow cytometry. In some embodiments, the level of the gRNA in each cell having a high expression of the reporter protein is determined by deep sequencing. In some embodiments, the gRNA increases the expression of the reporter protein in the cell by about 2-50% relative to a non-targeting gRNA.

### 9. EXAMPLES

### Example 1

### Materials and Methods

Construction of a TUBB3-2A-mCherry pluripotent stem cell line. A human iPS cell line (RVR-iPSCs) was used to construct the TUBB3-2A-mCherry reporter line. RVR-iPSCs were retrovirally reprogrammed from BJ fibroblasts and characterized as previously done (Lee et al. Cell 2012, 51, 547-558). To generate the TUBB3-2A-mCherry reporter line, 3×10⁶ cells were dissociated with Accutase (Stemcell Tech, 7920) and electroporated with 6 μg of gRNA-Cas9 expression vector and 3 μg of TUBB3 targeting vector using the P3 Primary Cell 4D-Nucleofector Kit (Lonza, V4XP-3032). Transfected cells were plated into a 10 cm dish coated with Matrigel (Corning, 354230) in compete mTesR (Stemcell Tech, 85850) supplemented with 10 μM Rock Inhibitor (Y-27632, Stemcell Tech, 72304). 24 hours after transfection, positive selection began with 1 μg/mL puromycin for 7 days. Following selection, cells were transfected with a CMV-CRE recombinase expression vector to remove the foxed puromycin selection cassette. Transfected cells were expanded and plated at low density for clonal isolation (180 cells/cm²). Resulting clones were mechanically picked and expanded and gDNA was extracted using QuickExtract DNA Extraction Solution (Lucigen, QE09050) for PCR screening of targeting vector integration. A second round of clonal isolation was performed using the same protocol following lentiviral transduction of ^{VP64}dCas9^{VP54}.

Plasmid construction. The lentiviral ^{VP64}dCas9^{VP64} plasmid was generated by modifying Addgene plasmid #59791 to replace GFP with the BSD blasticidin resistance gene. The lentiviral dSaCas9^{KRAB} plasmid was generated by modifying Addgene plasmid #106249 to insert a S. aureus gRNA cassette with a ZFP36L1. HES3 or scrambled non-targeting gRNA. The gRNA expression plasmid for the single CAS-TF screen was generated by modifying Addgene plasmid #83925 to contain an optimized gRNA scaffold (Chen et al, Cell 2013, 155, 1479-149) and a puromycin resistance gene in place of Bsr. The gRNA expression plasmids for the paired CAS-TF screens were generated by further modification of the single gRNA expression plasmid to contain an additional gRNA cassette expressing either sgNGN3 or sgASCL1 under control of the mU6 Pol III promoter with a modified gRNA scaffold described previously (Adamson et al. Cell 2016, 167, 1867-1882 e1821). Individual gRNAs were ordered as oligonucleotides (Integrated DNA Technologies), phosphorylated, hybridized, and cloned into the gRNA expression plasmids using BsmBI sites. Protospacers used for individual gRNA cloning are listed in TABLE 3, above.

The TUBB3 targeting vector was cloned by inserting ˜700 bp homology arms (surrounding the TUBB3 stop codon), amplified by PCR from genomic DNA of RVR-iPS cells, surrounding a P2A-mCherry sequence with a floxed puromycin resistance cassette.

cDNAs encoding TFs were either PCR amplified from cDNA pools or synthesized as gBlocks (Integrative DNA Technologies) and cloned into Addgene plasmid #52047 using EcoRI and XbaI restriction sites. TetO gene expression was achieved by co-delivery of M2rtTA (Addgene #20342).

Lentiviral production and titration. HEK293T cells were acquired from the American Tissue Collection Center (ATCC) and purchased through the Duke University Cell Culture Facility, The cells were maintained in DMEM High Glucose supplemented with 10% FBS and 1% penicillin-streptomycin and cultured at 37° C. with 5% CO2. For lentiviral production of the gRNA libraries, ^{VP64}dCas9^{VP64} and dSaCas9^{KRAB}, 4.5×10⁶ cells were transfected using the calcium phosphate precipitation method (Salmon and Trono, 2007 Curr. Proloc. Hum. Genet. Chapter 12, Unit 12 10) with 6 μg pMD2.G (Addgene #12259), 15 μg psPAX2 (Addgene #12260), and 20 μg of the transfer vector. The medium was exchanged 12-14 hours after transfection, and the viral supernatant was harvested 24 and 48 hours after this medium change, The viral supernatant was pooled and centrifuged at 600 g for 10 min, passed through a PVDF 0.45 μm filter (Millipore, SLHV033RB) and concentrated to 50× in 1× PBS using Lenti-X Concentrator (Clontech, 631232) in accordance with the manufacturer's protocol.

To produce lentivirus for gRNA and cDNA validations, 0.4×10⁶ cells were transfected using Lipofectamine 3000 (Invitrogen, L3000008) according to the manufacturer's instructions with 200 ng pMD2.G, 600 ng psPAX2, and 200 ng of the transfer vector. The medium was exchanged 12-14 hours after transfection, and the viral supernatant was harvested 24 and 48 hours after this medium change. The viral supernatant was pooled and centrifuged at 600 g for 10 min and concentrated to 50× in 1× PBS using Lenti-X Concentrator (Clontech, 631232) in accordance with the manufacturer's protocol.

The titer of the lentiviral gRNA library pools for the single or paired CAS-TF libraries was determined by transducing 6×10⁴ cells with serial dilutions of lentivirus and measuring the percent GFP expression 4 days after transduction with an Accuri C6 flow cytometer (BD). All lentiviral titrations were performed in the TUBB3-2A-mCherry cell line used in the CAS-TF single and paired gRNA screens.

CAS-TF gRNA library design and cloning. Putative TFs were selected from a previous catalog of human transcription factors (Vaquerizas et al. Nat. Rev. Genet 2009, 10, 252-263). A gRNA library consisting of 5 gRNAs per TSS targeting 1,496 TFs was extracted from a previous genorne-wide CRISPRa library (Horlbeck, 2016 Compact and highly active next-generation libraries. eLife). The library included a set of 100 scrambled non-targeting gRNAs extracted from the same genome-wide library for a total of 8,505 gRNAs. The oligonucleotide pool (Custom Array) was PCR amplified and cloned using Gibson assembly into the single gRNA expression plasmid for the single CAS-TF screen or the dual gRNA expression plasmid for the paired CAS-TF screens with sgASCL1 or sgNGN3.

The sub-library was designed by extracting additional gRNAs from several previously published CRISPRa genome-wide libraries (Gilbert et al. Cell 2014, 159, 647-66; Horlbeck, 2016 Compact and highly active next-generation libraries, eLife; Konermann et al. Nature 2015, 517, 583-588; Sanson et al. Nat. Commun, 2018, 9, 5416) to obtain an average of 33 gRNAs per gene targeting 109 TFs. The library included a set of 300 scrambled non-targeting gRNAs for a total of 3,874 gRNAs. The oligonucleotide pool (Twist Bioscience) was PCR amplified and cloned into the single gRNA expression plasmid as done with the original CAS-TF library.

Single and paired CAS-TF neuronal differentiation screens. Each CAS-TF screen was performed in triplicate with independent transductions. For each replicate, 24×10⁶ TUBB3-2A-mCherry ^{VP64}dCas9^{VP64}iPSCs were dissociated using Accutase (Stemcell Tech, 7920) and transduced in suspension across five matrigel-coated 15-cm dishes in mTesR (Stemcell Tech 85850) supplemented with 10 μM Rock Inhibitor (Y-27632, Stemcell Tech, 72304). Cells were transduced at a MOI of 0.2 to obtain one gRNA per cell and ˜550-fold coverage of the CAS-TF gRNA library. The medium was changed to fresh mTesR without Rock Inhibitor 18-20 hours after transduction. Antibiotic selection was started 30 hours after transduction by adding 1 μg/mL puromycin (Sigma, P8833) directly to the plates without changing the medium. 48 hours after transduction the medium was changed to neurogenic medium (DMEM/F-12 Nutrient Mix (Gibco, 11320), 1× B-27 serum-free supplement (Gibco, 17504), 1× N-2 supplement (Gibco, 17502), and 25 μg/mL gentamicin (Sigma, G1397) supplemented with 1 μg/mL puromycin for the remainder of the experiment with daily medium changes.

Cells were harvested for sorting 5 days after transduction of the gRNA library for the single factor CAS-TF screen and the sgASCL1 paired screen. Cells were harvested 4 days after transduction for the sgNGN3 paired screen. Cells were washed once with 1× PBS, dissociated using Accutase, filtered through a 30 μm CellTrics filter (Sysmex, 04-004-2326) and resuspended in FACS Buffer (0.5% BSA (Sigma, A7906), 2 mM EDTA (Sigma, E7889) in PBS). Before sorting, an aliquot of 4.8×10⁶ cells was taken to represent a bulk unsorted population. The highest and lowest 5% of cells were sorted based on mCherry expression and 4.8×10⁶ cells were sorted into each bin. Sorting was done with a SH800 FACS Cell Sorter (Sony Biotechnology). After sorting, genomic DNA was harvested with the DNeasy Blood and Tissue Kit (Qiagen, 69506).

Sub-library screen. The CAS-TF sub-library screen was performed in triplicate with independent transductions. For each replicate, 9.6×10⁶ TUBB3-2A-mCherry ^{VP64}dCas9^{VP64}iPSCs were dissociated using Accutase (Stemcell Tech, 7920) and transduced in suspension across two matrigel-coated 15-cm dishes in mTesR (Stemcell Tech 85850) supplemented with 10 μM Rock Inhibitor (Y-27632, Stemcell Tech, 72304). Cells were transduced at a MOI of 0.2 to obtain one gRNA per cell and ˜495-fold coverage of the CAS-TF gRNA sub-library. The medium was changed to fresh mTesR without Rock Inhibitor 18-20 hours after transduction. Antibiotic selection was started 30 hours after transduction by adding 1 μg/mL purornycin (Sigma, P8833) directly to the plates without changing the medium. 48 hours after transduction the medium was changed to neurogenic medium (DMEM/F-12 Nutrient Mix (Gibco, 11320), 1× B-27 serum-free supplement (Gibco, 17504), 1× N-2 supplement (Gibco, 17502), and 25 μg/mL gentamicin (Sigma, G1397)) supplemented with 1 μg/mL puromycin for the remainder of the experiment with daily medium changes.

Cells were harvested for sorting 5 days after transduction of the gRNA library. Cells were washed once with 1× PBS, dissociated using Accutase, filtered through a 30 μm CellTrics filter (Sysmex, 04-004-2326) and resuspended in FACS Buffer (0.5% BSA (Sigma, A7906), 2 mM EDTA (Sigma, E7889) in PBS). Before sorting, an aliquot of 2×10⁶ cells was taken to represent a bulk unsorted population. The highest and lowest 5% of cells were sorted based on mCherry expression and 2×10⁶ cells were sorted into each bin. Sorting was done with a SH800 FACS Cell Sorter (Sony Biotechnology). After sorting, genomic DNA was harvested with the DNeasy Blood and Tissue Kit (Qiagen, 69506).

gRNA library sequencing. The gRNA libraries were amplified from each genomic DNA sample across 100 μL PCR reactions using Q5 hot start polymerase (NEB, M0493) with 1 μg of genomic DNA per reaction. The PCR amplification was done according to the manufacturer's instructions, using 25 cycles at an annealing temperature of 60° C. with the following primers:

Fwd:
5′-AATGATACGGCGACCACCGAGATCTACACAATTTCTTGGGTAGTTT
GCAGTT
Rev:
5′-CAAGCAGAAGACGGCATACGAGAT-(6-bp index sequence)-
GACTCGGTGCCACTTTTTCAA

The amplified libraries were purified with Agencourt AMPure XP beads (Beckman Coulter, A63881) using double size selection of 0.65× and then 1× the original volume to purify the 282 by amplicon. Each sample was quantified after purification with the Qubit dsDNA High Sensitivity assay kit (Thermo Fisher, Q32854). Samples were pooled and sequenced on a MiSeq (Illumina) with 20-bp paired-end sequencing using the following custom read and index primers:

Read1:
(SEQ ID NO: 32)
5′-GATTTCTTGGCTTTATATATCTTGTGGAAAGGACGAAACACCG.
Index:
(SEQ ID NO: 33)
5′-GCTAGTCCGTTATCAACTTGAAAAAGTGGCACCGAGTC.
Read2:
(SEQ ID NO: 34)
5′-GTTGATAACGGACTAGCCTTATTTAAACTTGCTATGCTGTTTCCAG
CATAGCTCTTAAAC.

Data processing and enrichment analysis. FASTQ files were aligned to custom indexes of the 8,505 protospacers (generated from the bowtie2-build function) using Bowtie 2 (Langmead and Salzberg Nat. Methods 2012, 9, 357-359). Counts for each gRNA were extracted and used for further analysis. All enrichment analysis was done with R. Individual gRNA enrichment was determined using the DESeq2 (Love et al. Genome Biol. 2014, 15, 550) package to compare gRNA abundance between high and low, unsorted and low, or unsorted and high conditions for each screen. TFs were selected as hits if two or more gRNAs were significantly enriched (FDR<0.01) in the mCherry-high cell bin relative to both the unsorted and the mCherry-low cell bins.

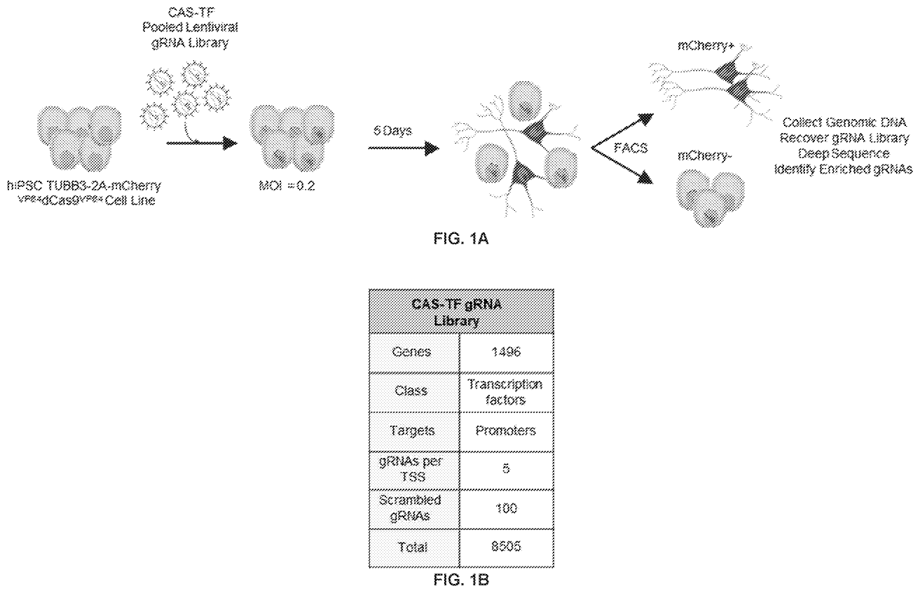

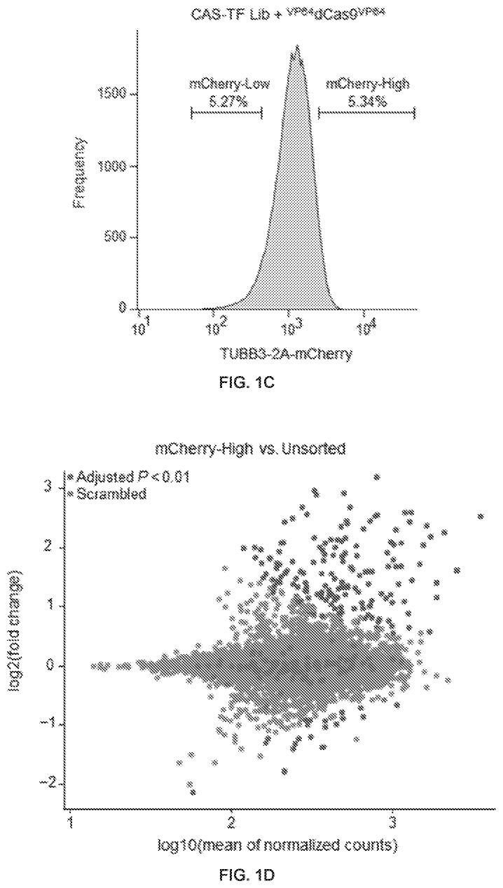

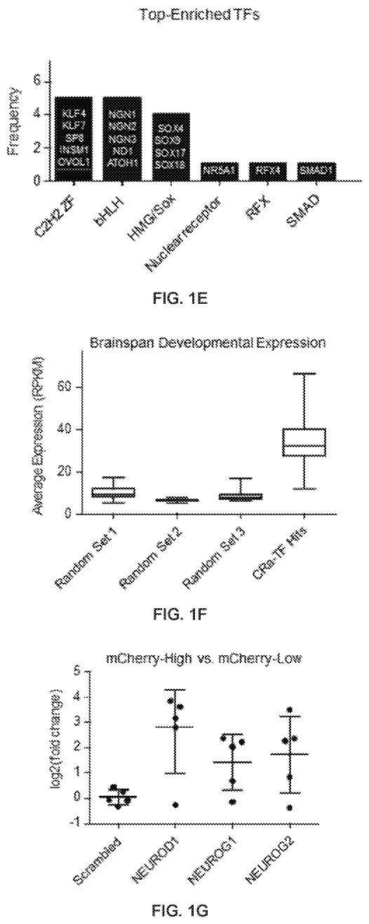

In vivo expression comparison. RNA-sequencing data generated as part of the Brainspan Developmental Transcriptome Atlas was downloaded (Miller et al. Nature 2014, 508, 199-206). The average expression for the 17 TFs identified in the single-factor CAS-TF screen was calculated for each developmental time point and anatomical region listed between 8 and 13 post conception weeks. A random set of 17 TFs was identically analyzed, and a representative comparison is shown in FIG. 1F.

gRNA and cDNA validations. The top enriched gRNAs from the screens were cloned into the appropriate gRNA expression vector as described previously. The gRNA validations were performed similarly as done with the screens, except the transductions were performed in 24-well plates and the virus was delivered at high MOI. Cells were harvested for flow cytometry or qRT-PCR 4 days after gRNA transduction.

For immunofluorescence staining experiments, the cDNAs encoding the top enriched TFs were PCR amplified and cloned into a doxycycline inducible expression vector as described previously. Cells were co-transduced in suspension with the indicated TFs along with a separate lentivirus encoding the M2rtTA (Addgene #20342) in mTesR supplemented with 10 pM Rock Inhibitor. Unmodified iPSCs were used for these experiments to enable staining with red fluorophores without interference from the mCherry reporter. 18-20 hours after transduction, the medium was changed to neurogenic medium supplemented with 0.1 μg/mL doxycycline (Sigma, D9891). Staining was done 4 days after transduction as described previously. For a subset of the TFs, the TUBB3-2A-mCherry cell line was used to sort off the highest mCherry expressing cells 3 days after transduction. The cells were replated onto a pre-established monolayer of human astrocytes (Lonza, CC-2565) and cultured for an additional 8 days in neurogenic medium before staining, gRNA and cDNA validations in H9 human embryonic stem cells were performed similarly to those described for iPSCs. A polyclonal ^{VP64}dCas9^{VP64}H9 ESC line was established via lentiviral transductions, and gRNAs were delivered with a separate lentivirus.

Quantitative RT-PCR. Cells were dissociated with Accutase (StemCell Tech, 7920) and centrifuged at 300 g for 5 min. Total RNA was isolated using RNeasy Plus (Qiagen, 74136) and QIAshredder kits (Qiagen, 79656). Reverse transcription was carried out on 0.1 μg total RNA per sample in a 10 μL reaction using the SuperScript VILO Reverse Transcription Kit (Invitrogen, 11754). 1.0 μL of cDNA was used per PCR reaction with Perfecta SYBR Green Fastmix (Quanta BioSciences, 95072) using the CFX96 Real-Time PCR Detection System (Bio-Rad). The amplification efficiencies over the appropriate dynamic range of all primers were optimized using dilutions of purified amplicon. All amplicon products were verified by gel electrophoresis and melting curve analysis. All qRT-PCR results are presented as fold change in RNA normalized to GAPDH expression. Primers used in this study can be found in TABLE 4.

TABLE 4
All qRT-PCR primers used in this study.

| Gene | Primer Sequence | SEQ ID NO |
|---|---|---|
| NCAM Fwd | AACCCAGTGCACCTAAGCTC | 105 |
| NCAM Rev | GGACTTCAGCATGACGTGGT | 106 |
| MAP2 Fwd | CAGCTTGTCTCTAACCGAGGA | 107 |
| MAP2 Rev | TGTGTCGTGTTCTCAAAGGGT | 108 |
| TUBB3 Fwd | TTTGGACATCTCTTCAGGCC | 109 |
| TUBB3 Rev | TTTCACACTCCTTCCGCAC | 110 |
| ZFP36L1 Fwd | CCGAGTCCCCTCACATGTTT | 111 |
| ZFP36L1 Rev | TTGAGTTGTCCAAGGTCGGG | 112 |
| HES3 Fwd | GAAAGTCTCCCTGGCTCGTC | 113 |
| HES3 Rev | CCAAATAGGGAGCGCCTTCA | 114 |
| NEUROG3 Fwd | TTTTCTCCTTTGGGGCTGGG | 115 |
| NEUROG3 Rev | AGGCGTCATCCTTTCTACCG | 116 |
| RFX4 Fwd | GACGAGCGGCCATTCATCAG | 117 |
| RFX4 Rev | CACTCAGTAATCCAGCCGGG | 118 |
| SOX4 Fwd | AACAGGGCGGCTGGTTAATA | 119 |
| SOX4 Rev | ACACTGGTGGCAGGTTAAGG | 120 |
| NEUROD1 Fwd | GATGACTAAGGCTCGCCTGG | 121 |
| NEUROD1 Rev | AGAATAGCAAGGCACCACCT | 122 |
| INSM1 Fwd | TACGCGTTTGTCTCGTGGTT | 123 |
| INSM1 Rev | CAGAGATTGGTAGGCGAGGC | 124 |
| KLF7 Fwd | TTGCATTAGGAGCGAACAGC | 125 |
| KLF7 Rev | AAAAGGGGACTTCTCCACGG | 126 |
| SOX9 Fwd | TAAAACGGTGCTGCTGGGAA | 127 |
| SOX9 Rev | AGTGTGCTCGGGCACTTATT | 128 |
| SOX17 Fwd | GACATGAAGGTGAAGGGCGA | 129 |
| SOX17 Rev | CGTTGTGCAGGTCTGGATTC | 130 |
| NEUROG1 Fwd | AATATCTCCCGGGCGTCTGA | 131 |
| NEUROG1 Rev | GTTCAAGTTGTGCATGCGGT | 132 |
| SP8 Fwd | CTTCTAGGGGAAGAACCGAGG | 133 |
| SP8 Rev | AAGAGGACGAGGAGCGTTTC | 134 |
| KLF4 Fwd | CACCGGACCTACTTACTCGC | 135 |
| KLF4 Rev | AACCCCAAATTGGCCGAGAT | 136 |
| SMAD1 Fwd | GGAGAAAGGAGAGGCCGAGC | 137 |
| SMAD1 Rev | AAAAGTAACCCAGTCAGCACCG | 138 |
| OVOL1 Fwd | GTCCGGCTCGCACTTTAAGA | 139 |
| OVOL1 Rev | CTGAGAACGAGGTCCCTTGC | 140 |
| NR5A1 Fwd | GTGGTGTGAGGGGGTTTCTG | 141 |
| NR5A1 Rev | TACGAATAGTCCATGCCCGC | 142 |
| ATOH1 Fwd | AGGATGCATGGGCTGAACC | 143 |
| ATOH1 Rev | TTGTAGCAGCTCGGACAAGG | 144 |
| NEUROG2 Fwd | CAGGCCAAAGTCACAGCAAC | 145 |
| NEUROG2 Rev | CGATCCGAGCAGCACTAACA | 146 |
| SOX18 Fwd | GCAAAGGACGAGCGCAAG | 147 |
| SOX18 Rev | CTTGTAGTTGGGGTGGTCGC | 148 |
| SOX11 Fwd | AGCGGAGGAGGTTTTCAGTG | 149 |
| SOX11 Rev | TTCCATTCGGTCTCGCCAAA | 150 |

Immunofluorescence staining. Cells were washed briefly with PBS and then fixed with 4% paraformaldehyde (Santa Cruz, sc-281692) for 20 minutes at room temperature. Cells were washed twice with PBS and then incubated with blocking buffer (10% goat serum (Sigma, G6767), 2% BSA (Sigma, A7906) in PBS) for 30 min at room temperature. Cells were permeabilized with 0.2% Triton-X 100 (Sigma, T8787) for 10 min at room temperature. The following primary antibodies were used with incubations for 2 hours at room temperature: Mouse anti-TUBB3 (1:1000 dilution, BioLegend, 801201); Rabbit anti-MAP2 (1:500 dilution, Sigma, AB5622). Cells were washed three times with PBS and then incubated with secondary antibody and DAPI (Invitrogen, D3571) in blocking solution for 1 hour at room temperature. The following secondary antibodies were used: Alexa Fluor 488 goat anti-mouse (1:500 dilution, Invitrogen, A-11001); Alexa Fluor 594 goat anti-rabbit (1:500 dilution, Invitrogen, A-11012). Cells were washed three times with PBS and imaged with a Zeiss 780 upright confocal microscope.

For NCAM staining of live cells for gRNA validations, cells were dissociated with Accutase (Sterncell Tech, 7920), centrifuged at 300g for 5 min, and resuspended in staining buffer (0.5% BSA (Sigma, A7906) and 2 mM EDTA (Sigma, E7889) in PBS) at 10×10⁶ cells per mL. Mouse anti-CD56 (NCAM, Invitrogen, 12-0567) was added at 0.6 pg per 1×10⁶ cells and incubated for 30 min at 4° C. Cells were washed with 1 mL staining buffer, centrifuged at 300 g for 5 min and resuspended in staining buffer for analysis on the SH800 FACS Cell Sorter (Sony Biotechnology).

RNA-sequencing with tetO cDNA expression. TUBB3-2A-mCherry iPSCs were co-transduced with a lentivirus encoding M2rtTA and the indicated tetO-cDNA. Cells were transduced in mTesR with 10 μM Rock Inhibitor. The following day, the medium was changed to neurogenic medium (DMEM/F-12 Nutrient Mix (Gibco, 11320), 1× B-27 serum-free supplement (Gibco, 17504), 1× N-2 supplement (Gibco, 17502), and 25 μg/mL gentamicin (Sigma, G1397)) supplemented with 0.1 μg/mL doxycycline. Cells were sorted after 2 or 3 days of transgene expression using a SH800 FACS Cell Sorter in semi-purity mode. Sorted cells were replated onto matrigel-coated 24-well plates and cultured in neurogenic medium supplemented with 10 ng/mL each of BDNF, GDNF and NT-3 (PeproTech) until harvest after 6 or 7 days.

Total RNA was extracted using RNeasy Mini Kit (Qiagen) and 100 ng of RNA was used to develop RNA-seq libraries. RNA-sequencing libraries were prepared using the Truseq Stranded mRNA kit (Illumina) according to the manufacturer's protocol. The libraries were sequenced on a NextSeq 500 on High Output Mode with 75 bp paired-end reads. Reads were first trimmed using Trimmomatic v0.32 to remove adapters and then aligned to GRCh38 using STAR aligner (Langmead et al. Nat. Methods 2012, 9, 357-359). Gene counts were obtained with featureCounts from the subread package (version 1.4.6-p4) using the comprehensive gene annotation in Gencode v22. Differential expression analysis was determined with DESeq2 where gene counts are fitted into negative binomial generalized linear models (GLMs) and Wald statistics determine significant hits. Genes were included for analysis if at least three samples across all conditions tested had a TPM>1. Gene Ontology analyses were performed using the Gene Ontology Consortium database (Ashburner at al., 2000, The Gene Ontology Consortium, 2017) and Synaptic Gene Ontology Consortium database (Koopmans et al. Neuron 2019, 103, 217-234 e214).

Electrophysiology. TUBB3-2A-mCherry iPSCs were co-transduced with a lentivirus encoding M2rtTA and either tetO-NEUROG3 alone or in combination with tetO-LHX8. Cells were transduced in mTesR with 10 μM Rock Inhibitor. The following day, the medium was changed to neurogenic medium supplemented with 0.1 μg/mL doxycycline. Cells were sorted after 3 days of transgene expression using a SH800 FACS Cell Sorter in semi-purity mode. Sorted cells were replated onto rnatrigel-coated coverslips and cultured in neurogenic medium supplemented with 10 ng/mL each of BDNF, GDNF and NT-3 (PeproTech) for the remainder of the experiment.

Whole-cell patch-clamp recordings were performed on cultured cells 7 days post-induction of transgene expression under a Zeiss Axio Examiner.D1 microscope. To avoid osmotic shock, culture media was gradually changed to artificial CSF (aCSF) in a step-wise manner over approximately 5 minutes, and then the coverslip was moved to the recording chamber, aCSF contained 124 mM NaCl, 26 mM NaHCO₃, 10 mM D-glucose, 2 mM CaCl₂, 3 mM KCl, 1.3 mM MgSO₄, and 1.25 mM NaH₂PO₄ (310 mOsm/L) and was continuously bubbled at room temperature with 95% O₂ and 5% CO₂. Cells were inspected under a 20× water-immersion objective using infrared illumination and differential interference contrast optics (IR-DIC). The experimenter was blinded to the condition and chose the most morphologically complex neurons for recording. Electrodes (4-7 MΩ) were pulled from borosilicate glass capillaries using a P-97 puller (Sutter Instrument) and filled with an intracellular solution containing 135 mM K-methanesulfonate, 8 mM NaCl, 10 mM HEPES, 0.3 mM EGTA, 4 mM MgATP, and 0.3 mM Na₂GTP (pH 7.3 with KOH, adjusted to 295 mOsm/L with sucrose), Alter gigaohm seals were ruptured, membrane resistance was measured in voltage-clamp mode with a brief hyperpolarizing pulse, and membrane capacitance was estimated from the capacitance compensation circuitry of the amplifier. Then, resting membrane potential was recorded in current-clamp mode. Finally, a small holding current was applied to adjust the membrane potential to around −60 mV, and input-output curves were generated by injecting increasing amounts of current. Data were recorded with a Multiclamp 700B amplifier (Molecular Devices) and digitized at 50 kHz with a Digidata 1550 (Molecular Devices). Action potential properties were calculated based on the first action potential generated using custom MATLAB scripts. Action potentials were counted by visual inspection if they had the characteristic two-component rising phase, regardless of peak amplitude. All experiments were analyzed blinded to the condition, and only recordings which remained stable over the entire period of data collection were used.

Orthogonal CRISPR-based gene regulation. TUBB3-2A-mCherry ^{VP64}dCas9^{VP64}iPSCs were transduced with an all-in-one dSaCas9^{KRAB} lentivirus (Thankore et al. Nat, Commun. 2018, 9, 1674) containing either a ZFP36L1, HES3 or scrambled S. aureus gRNA. After 2 days, antibiotic selection was started with 0.5 μg/mL puromycin, and cells were cultured for an additional 7 days in mTesR. After 9 days following transduction with dSaCas9^{KRAB} and S. aureus gRNAs, cells were transduced with a lentivirus encoding either sgNGN3 or sgASCL1 and switched to neurogenic medium. Cells were harvested 3 days after gRNA transduction for mRNA-sequencing and 4 days after gRNA transduction for flow cytometry.

Total RNA was isolated using RNeasy Plus (Qiagen, 74136) and QIAshredder kits (Qiagen, 79656). Libraries were prepped and sequenced by Genewiz on an Illumina Hiseq with 2×150 bp paired-end reads. The mean quality score for the sequencing run was 39.03 with 94.48% reads 30. The average number of reads per sample was ˜50,000,000 reads. mRNA-sequencing analysis was done as described previously for the tetO cDNA experiments. GFP transgene expression was quantified using bowtie2 to align trimmed reads to a custom GFP index generated with the bowtie2-build function. Raw counts were normalized for sequencing depth and displayed as relative counts across the three conditions analyzed.

Statistical methods. Statistical analysis was done using GraphPad Prism 7. See figure legends for details on specific statistical tests run for each experiment. Statistical significance is represented by a star (*) and indicates a computed p value<0.05.

### Example 2

### Generation of a Human Pluripotent Stem Cell Line for CRISPRa Screening of Neuronal Cell Fate

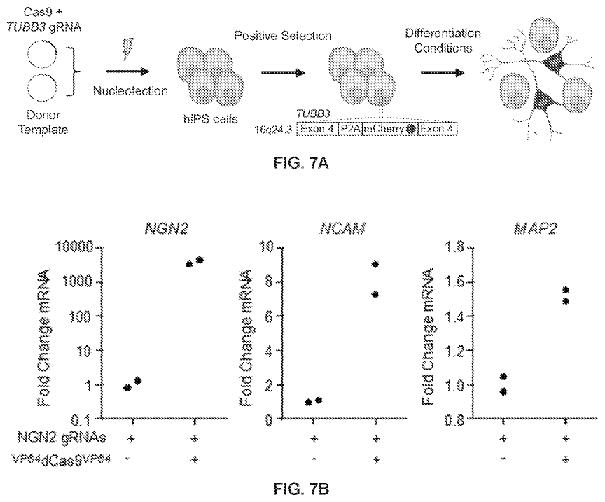

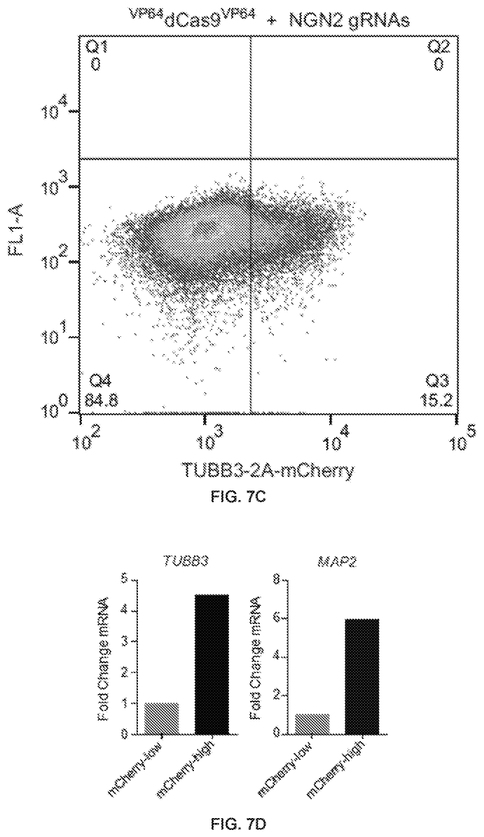

To enable the enrichment of neuronal cells within a CRISPRa screening framework, we inserted a 2A-mCherry sequence into exon 4 of the pan-neuronal marker TUBB3 in a human pluripotent stem cell line (FIG. 7A). TUBB3 is expressed almost exclusively in neurons and is induced early upon the in vitro differentiation and reprogramming of cells to neurons. The 2A-mediated ribosomal skipping ensures that mCherry serves as a translational reporter of TUBB3, while also mitigating any interference with endogenous TUBB3 function that might arise from a direct protein fusion.

To enable efficient and robust targeted gene activation in our TUBB3-P2A-mCherry cell line, we used a lentiviral vector to establish a clonal cell line expressing dCas9 fused to a VP64 transactivation domain at both its N- and C-termini (^{VP64}dCas9^{VP64}) under the control of the human ubiquitin C promoter (Kabadi et al. Nucleic Acids Res. 2014, 42, e147). ^{VP64}dCas9^{VP64} has been used previously to achieve robust endogenous gene activation sufficient for cell fate reprogramming.

To evaluate a CRISPRa approach for neuronal differentiation in our ^{VP64}dCas9^{VP64} TUBB3-2A-mCherry cell line, we delivered a pool of four lentiviral gRNAs targeting the proximal promoter of NEUROG2, a master regulator of neurogenesis sufficient to generate neurons from pluripotent stem cells when overexpressed ectopically or when activated endogenously with CRISPRa (Chavez et al. Nat. Methods 2015, 12, 326-328; Zhang et al. Neuron 2013, 78, 785-798). After five days of gRNA expression, we detected upregulation of the target gene NEUROG2, as well as of the early pan-neuronal markers NCAM and MAP2 (FIG. 7B), Targeted gene activation was only achieved if both ^{VP64}dCas9^{VP64} and NEUROG2 gRNAs were co-expressed (FIG. 7B).

Following delivery of NEUROG2 gRNAs, we detected 15% mCherry-positive cells relative to untreated control cells six days after transduction (FIG. 7C). To assess the applicability of our TUBB3-2A-mCherry reporter cell line as a proxy for a neuronal phenotype, we used fluorescent activated cell sorting (FACS) to isolate the highest and lowest 10% mCherry-expressing cells. The mCherry-high cells also had higher mRNA expression levels of the mCherry-tagged gene TUBB3, as well as MAP2 (FIG. 7D). The TUBB3-2A-mCherry cells and CRISPRa approach were used in all screens described in this study.

### Example 3

### CRISPRa Screen for Master Regulators of Neuronal Cell Fate

To identify a set of neuronal cell fate regulators in an unbiased manner, we performed a CRISPRa pooled gRNA screen in the TUBB3-2A-mChetry cell line (FIG. 1A), The gRNA library consisted of gRNAs targeting a set of putative human TFs (Vaquerizas et al, Nat. Rev. Genet. 2009, 10, 252-263). TFs are essential for cell-fate specification and have been applied extensively for cell reprogramming and directed differentiation applications. We selected a set of 1,496 TFs and constructed a targeted gRNA library of 5 gRNAs for each transcription start site, extracted from a genome-wide library of optimized CRISPRa gRNAs (Horlbeck, 2016, Compact and highly active next-generation libraries. eLife) (FIG. 1B).

The CRISPRa-TF gRNA lentiviral library (named CRISPR-Activation Screen TF, or CAS-TF) was transduced at a multiplicity of infection (MOI) of 0.2 and at 550-fold coverage of the library to ensure that most cells activated a single TF and to account for the stochastic and often inefficient nature of in vitro cell differentiations (FIG. 1A). After five days of gRNA expression, we used FACS to isolate the top and bottom 5% of mCherry-expressing cells (FIG. 1C) and quantified gRNA abundance with differential expression analysis following deep sequencing of the protospacers within each sorted bin. We collected the 5% tails of the mCherry distribution to enable the identification of subtle changes to TUBB3 expression. Cells were sorted on day five post-transduction to permit sufficient time for TF expression and induction of the reporter gene, while limiting the loss of post-mitotic neurons with extended time in culture or through passaging.

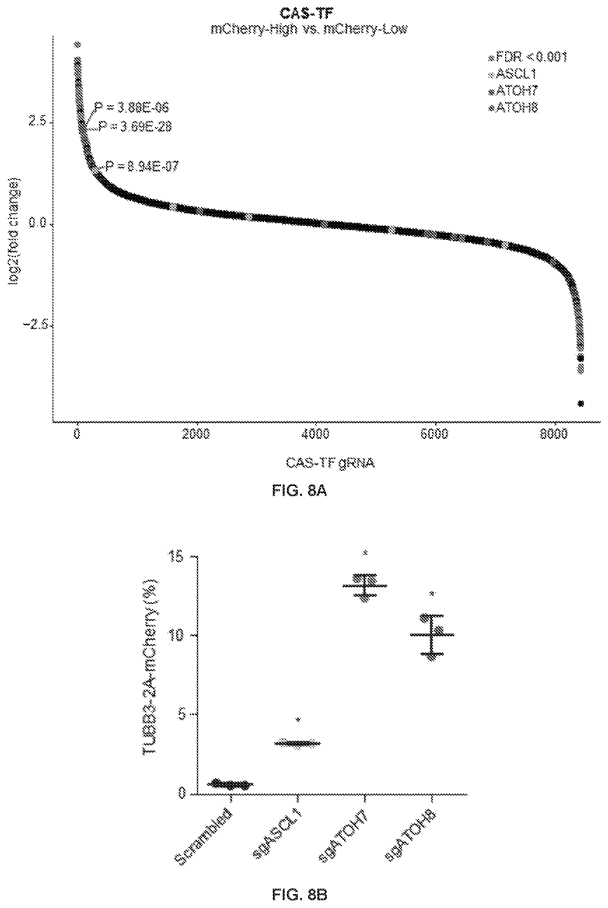

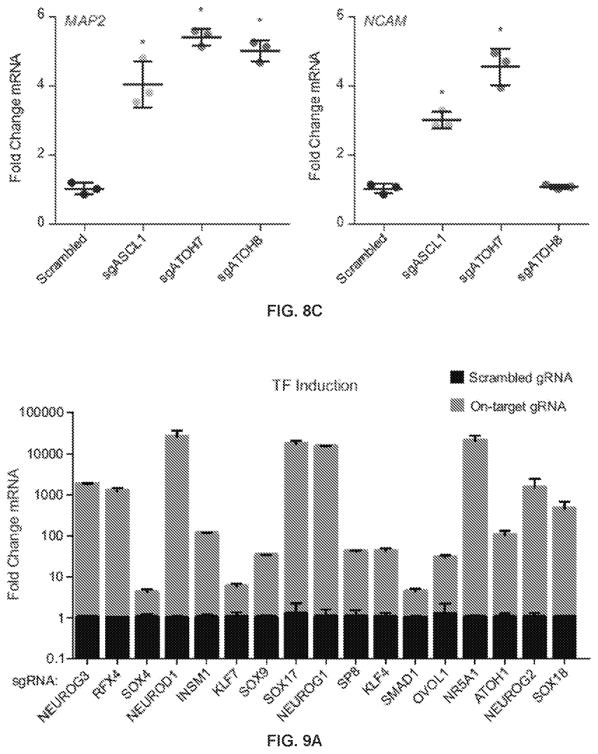

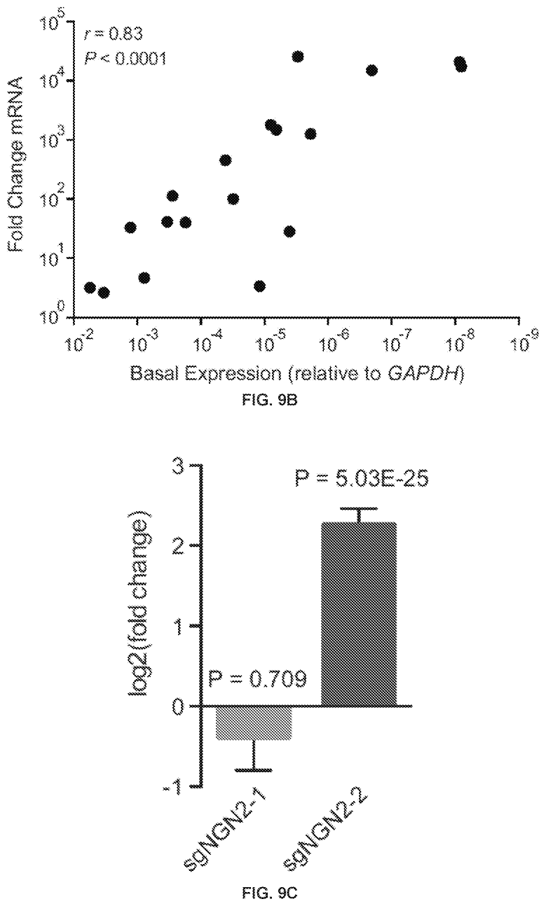

Compared to a bulk unsorted population of cells, there were gRNAs significantly enriched in the mCherry-high expressing cell bin (FDR<0.01; FIG. 1D). We observed similar results when comparing mCherry-high to mCherry-low expressing cells (FIG. 8A). A set of 100 scrambled non-targeting gRNAs were unchanged between the different cell bins (FIG. 1D).

The degree of transcriptional activation achieved with dCas9-based activators can vary across a set of gRNAs for a given target gene. As a consequence, we expected to observe a mixture of active and inactive gRNAs for most target genes. Additionally, off-target gRNA activity could promote false positives by modulating reporter gene expression independent of the predicted TF target. To ensure we did not over-interpret the results of a single gRNA, TFs were selected as high-confidence hits if they had at least two gRNAs significantly enriched in the mCherry-high expressing cell bin relative to both the unsorted and the mCherry-low cell bins (FDR<0.01). This approach yielded a list of 17 TFs as candidate neurogenic factors (FIG. 1E). The majority of these TFs belonged to either C2H2 ZF, bHLH, or HMG/Sox DNA-binding domain families, three of the most abundant families across all human transcription factors (FIG. 1E).

We analyzed the expression of the 17 candidate neurogenic factors with publicly available gene expression data in the developing human brain curated as part of BrainSpan (Miller et al. Nature 2014, 508, 199-206)(http://brainspan.org). We observed that the mean expression of the 17 factors, calculated across several anatomical regions and developmental time points of the human brain (see Example 1), was higher than that of a randomly generated set of 17 TFs (FIG. 1F).

As a further demonstration of the fidelity of the CAS-TF screen, we observed that three well-characterized proneural factors, NEUROD1, NEUROG1, and NEUROG2, each had several gRNAs enriched in mCherry-high expressing cells, while a random set of five scrambled non-targeting gRNAs was unchanged (FIG. 1G). A fourth gene with expected pro-neural activity, ASCL1, was not selected as a high-confidence hit based on our stringent selection criteria. However, a single ASCL1 gRNA was enriched in the mCherry-high expressing cells (FIG. 8A), and this gRNA was sufficient to generate mCherry-positive cells expressing NCAM and MAP2 (FIG. 8B and FIG. 8C).

### Example 4

### Validations of Candidate Neurogenic Transcription Factors

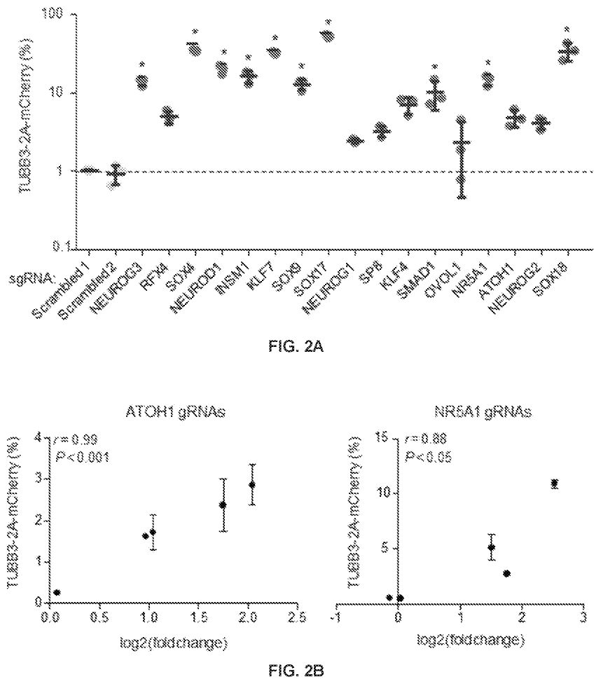

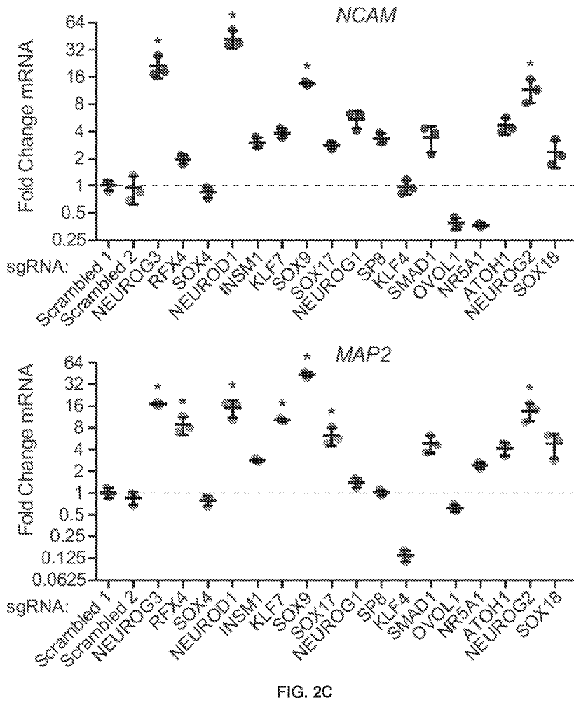

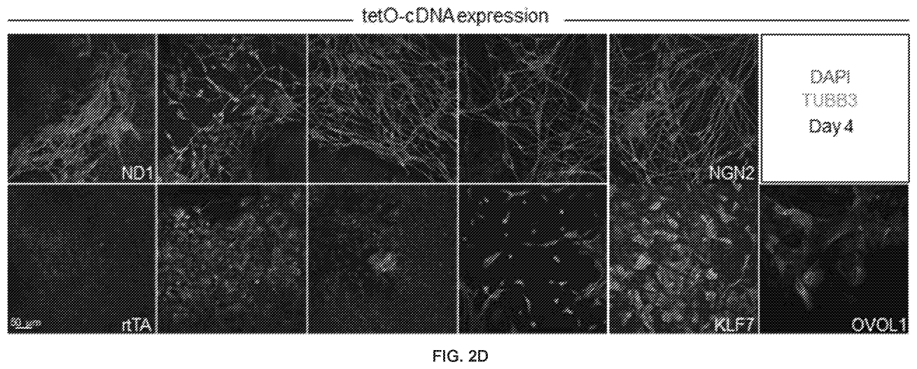

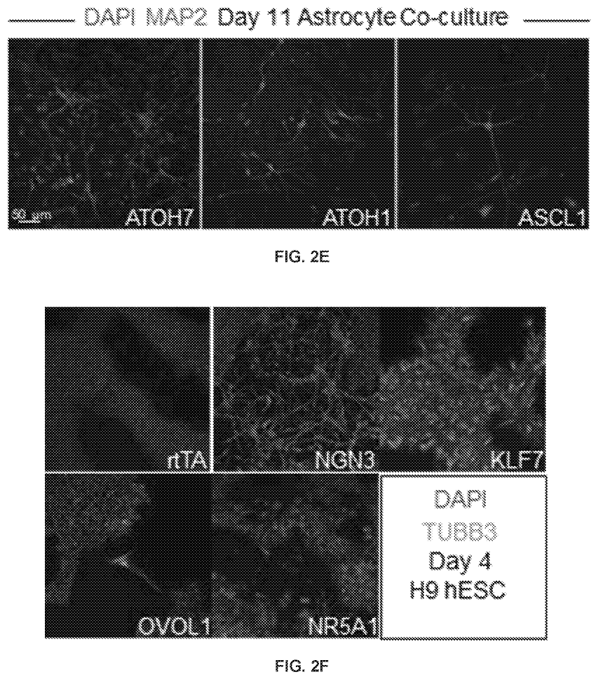

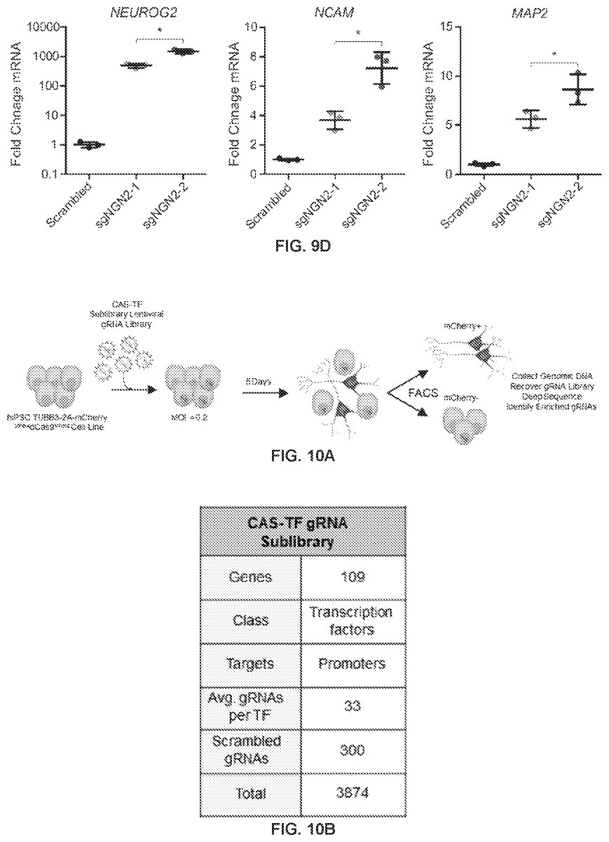

To validate the activity of the candidate neurogenic IFs, we individually tested the most enriched gRNA for the 17 IFs identified in the CAS-TF screen. We transduced these gRNAs at high MOI into the TUBB3-2A-mCherry cell line and evaluated reporter expression after four days (FIG. 2A). All of the gRNAs tested increased the number of mCherry-positive cells to varying degrees (from ˜2% to ˜50%) relative to the delivery of a scrambled non-targeting gRNA, although only a subset of 10 factors did so with statistical significance (FIG. 2A; α=0.05). To verify CRISPRa activity, we confirmed that all of the TFs were upregulated in response to expression of the appropriate gRNA (FIG. 9A). The degree of TF induction directly correlated with the basal expression level of the target gene, consistent with previous reports (Konerman Nature 2015, 517, 583-588) (FIG. 9B).

Further validations of all five gRNAs represented in the CAS-TF library for ATOH1 and NR5A1 revealed a direct correlation between the calculated enrichment from the pooled screen and the degree of differentiation assessed with reporter gene expression when the gRNAs were tested individually (FIG. 2B). In some cases, gRNAs that were not significantly enriched in the screen were still capable of modest gene activation and neuronal induction (FIG. 9C and FIG. 9D). For instance, a NEUROG2 gRNA was sufficient to upregulate NEUROG2, which was paralleled by NCAM and MAP2 induction, but was not enriched in the CAS-TF screen (FIG. 9C and FIG. 9D).

Given that we relied on a single reporter gene as a proxy for a neuronal phenotype, we expected that the TFs enriched in the CAS-TF screen would include both master regulators of neuronal fate sufficient to initiate differentiation, as well as cofactors or downstream effectors that only regulate one or a subset of neuronal genes. To clarify these differences within our set of candidate factors, we first evaluated the expression of two other neuronal markers, NCAM and MAP2, four days after gRNA delivery. Several TFs upregulated one or both of these markers, while other TFs generated no change or even downregulation (FIG. 2C), For instance, SOX4, which induced one of the largest increases in percent mCherry expression at an average of 34%, had no detectable effect on NCAM and MAP2 expression (FIG. 2A and FIG. 2C).

We used immunofluorescence staining to evaluate the presence of neuronal morphologies with expression of a subset of the TFs identified in our CAS-TF screen (FIG. 2D). To ensure robust TF expression and to control for differential gRNA activity, we overexpressed cDNAs encoding each TF. Several of the factors, including NEUROG3 and NEUROD1, generated cells with complex dendritic arborization that stained positively for TUBB3 within four days of expression (FIG. 2D), In contrast, many TFs upregulated TUBB3 as expected, but failed to generate cells with neuronal morphologies. We reasoned that the lack of morphological development in these cells could be attributable to slower differentiation kinetics. Other neuronal reprogramming paradigms often require extended culture to achieve morphological maturation. To account for this, we further cultured the cells for 11 days with primary astrocytes and found that with extended culture time, ATOH1, ATOH7, and ASCL1 were sufficient to generate cells with complex neuronal morphologies that stained positively for MAP2 (FIG. 2E). We did not observe similar morphological maturation with prolonged culture for KLF7_{;} NR5A1, and OVOL1.

To account for variation in response to expression of these TFs across different pluripotent stem cell lines, and to see if the lack of complete neuronal differentiation for several factors was a cell-line specific phenomenon, we also tested KLF7, NR5A1, and OVOL1 in H9 embryonic stem cells. We similarly observed a clear up-regulation of TUBB3 without the development of neuronal morphologies (FIG. 2F). As expected, NEUROG3 was able to induce rapid differentiation with the development of clear neuronal morphologies.

While the 17 high-confidence TF hits had a high validation rate, we suspected that many pro-neural TFs, similar to ASCL1, did not meet our stringent cutoff criteria, In fact, there were 109 other TFs that contained at least a single gRNA significantly enriched in the mCherry-high expressing cells but were not called as a hit. To further investigate these TFs, we first focused on TFs who shared a subfamily with one of the 17 high-confidence hits. For instance, ATOH1 was a high-confidence hit with several enriched gRNAs, however ATOH7 and ATOH8 both had only a single enriched gRNA (FIG. 8A). When these gRNAs were tested individually, ATOH7 and ATOH8 were both sufficient to generate mCherry-positive cells expressing NCAM and/or MAP2 (FIG. 8B and FIG. 8C), indicating that many hits with only single enriched gRNAs by this cutoff represent true positives.

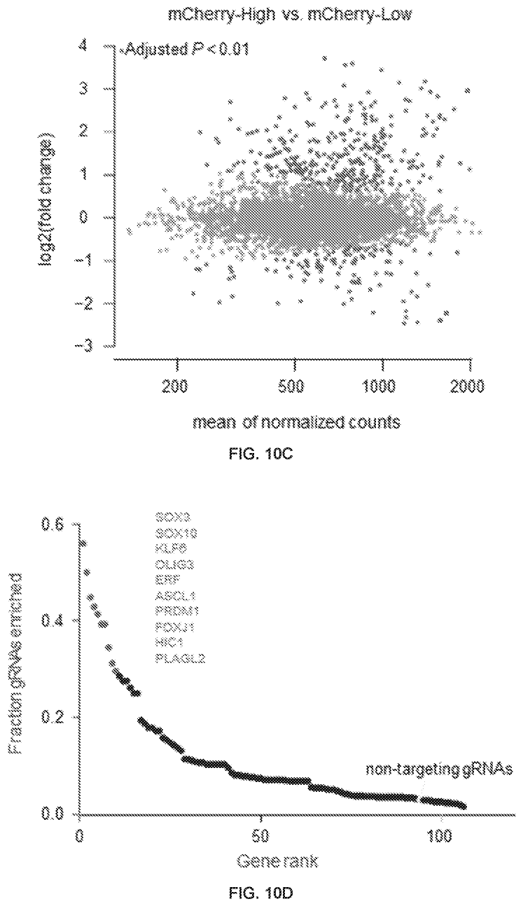

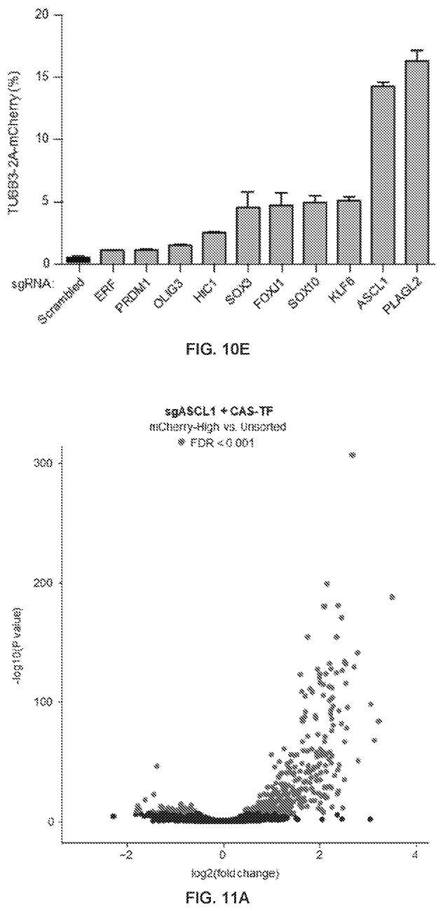

In order to more comprehensively validate the activity of these 109, we performed a secondary sub-library screen targeting only these TFs (FIG. 10A-FIG. 10E). This screen was performed in an identical fashion to the first CAS-TF screen (FIG. 10A), but the new sub-library consisted of an average of 33 gRNAs per TF (FIG. 10B), This screen revealed additional gRNAs enriched in mCherry-high cells (FIG. 10C). However, the majority of genes in the sub-library had relatively few enriched gRNAs, similar to a pool of scrambled non-targeting gRNAs (FIG. 10D). A few genes had over 40% of gRNAs enriched in the mCherry-high bin. However, individual validations of these gRNAs revealed mostly subtle effects on the mCherry reporter (FIG. 10E). This analysis both informs the design of robust CRISPRa screens and confirms that our screen design was successful in identifying the most robust neurogenic factors.

### Example 5

### Combinatorial gRNA Screens Identify Neuronal Cofactors

TFs often function cooperatively to orchestrate gene expression programs. Similarly, TF-mediated cell reprogramming often benefits from the co-expression of combinations of TFs to improve conversion efficiencies, maturation, and subtype specification. Because the mechanisms underlying the improvements observed with co-expressed IFs are often unknown, and because effective cofactors can have minimal activity when expressed alone, it can be challenging to predict effective TF cocktails. To address this challenge, we performed pooled screens with pairs of gRNAs to identify novel combinations of regulators that modulate neuronal differentiation of human pluripotent stem cells.

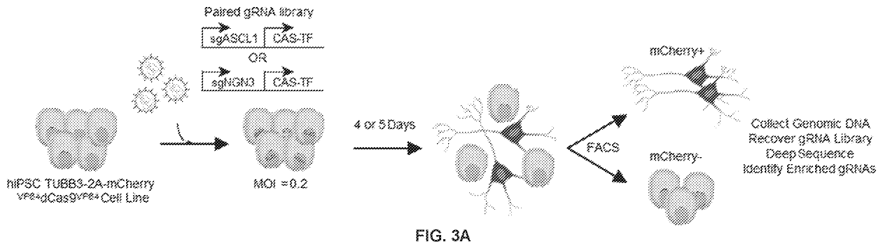

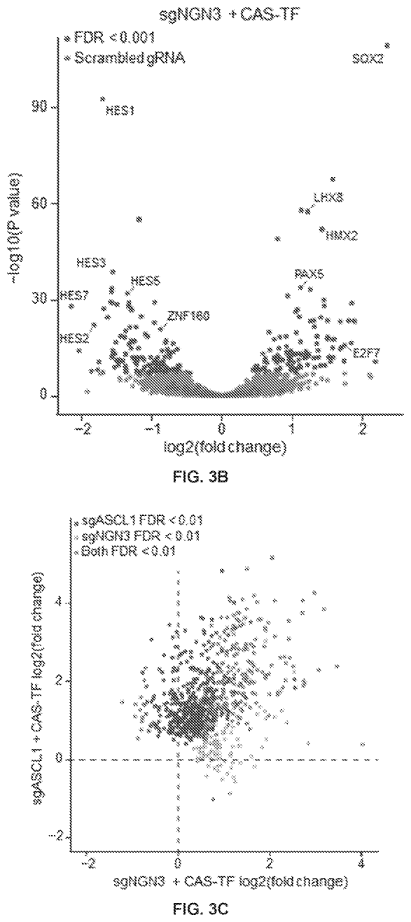

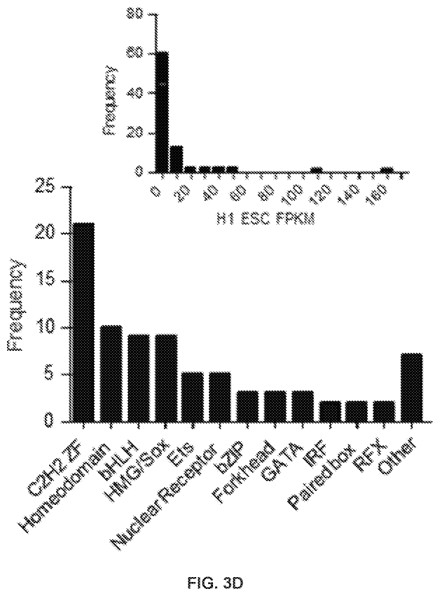

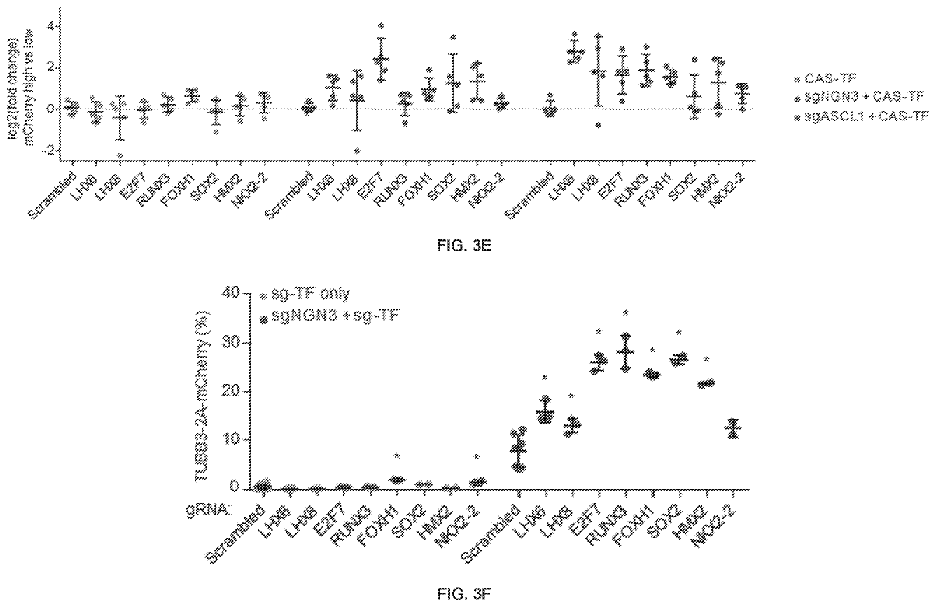

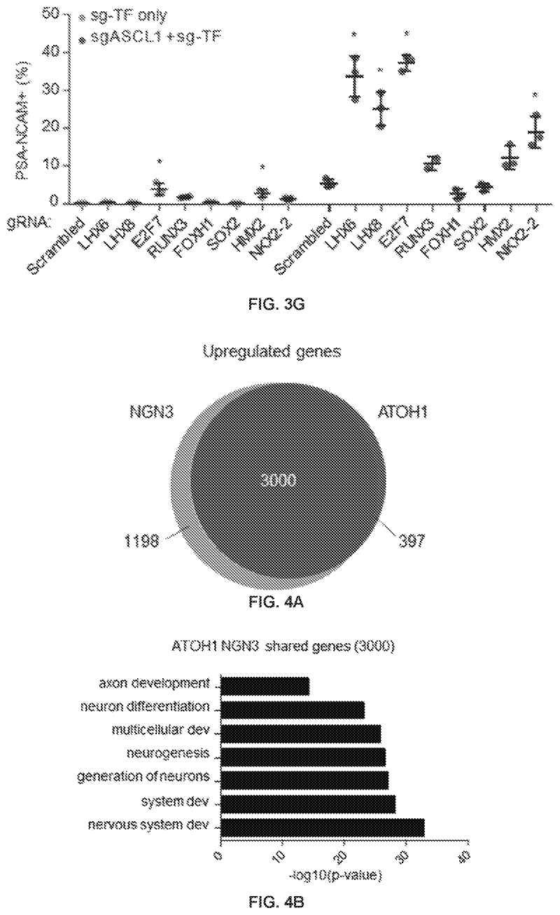

We hypothesized that some co-regulators of neuronal differentiation would lack detectable activity when expressed on their own, and thus would not be identified in our initial single-factor CAS-TF screen. Rather, these cofactors might require pairing with another neurogenic factor to reveal their activity. To enable the identification of such TFs, we opted to perform screens pairing a validated neurogenic TF identified from the single-factor screen with the remaining CAS-TF library (FIG. 3A), Two such independent screens were performed with a single gRNA for either NEUROG3 (sgNGN3) or ASCL1 (sgASCL1) (FIG. 3A). A pair of gRNAs was co-expressed on a single lentiviral vector from two independent RNA polymerase III promoters in a format adapted from previous studies (Adamson et al. Cell 2016, 167, 1867-1882 e1821). NEUROG3 and ASCL1 were chosen due to their strong neurogenic activity but differing kinetics of differentiation (FIG. 2D and FIG. 2E). The paired screens were performed as described for the single-factor screen, with each cell now receiving a single pair of gRNAs.

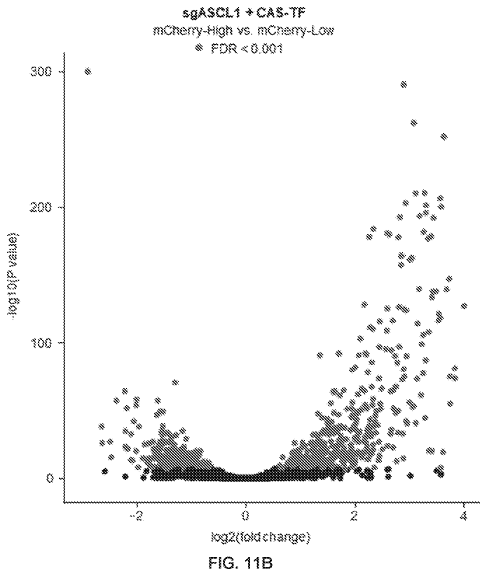

Due to the constitutive presence of a validated neurogenic factor in each cell, a clear mCherry-positive cell population emerged. Because of this basal neurogenic stimulus, in addition to the detection of novel positive cofactors of differentiation, we were also able to readily detect negative regulators in the mCherry-low expressing cells (FIG. 3B and FIG. 11A and FIG. 11B).

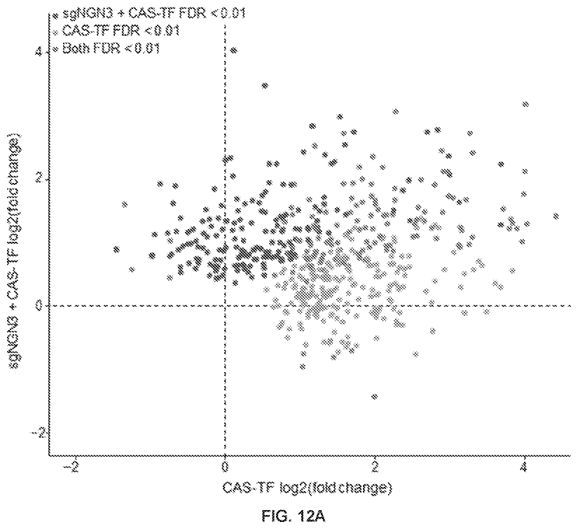

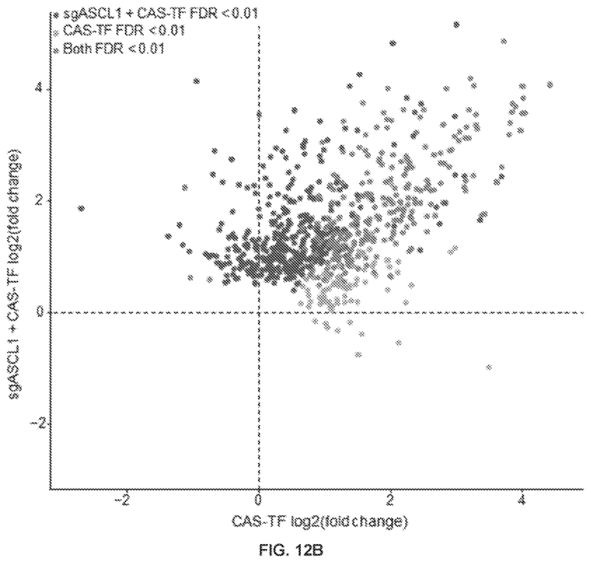

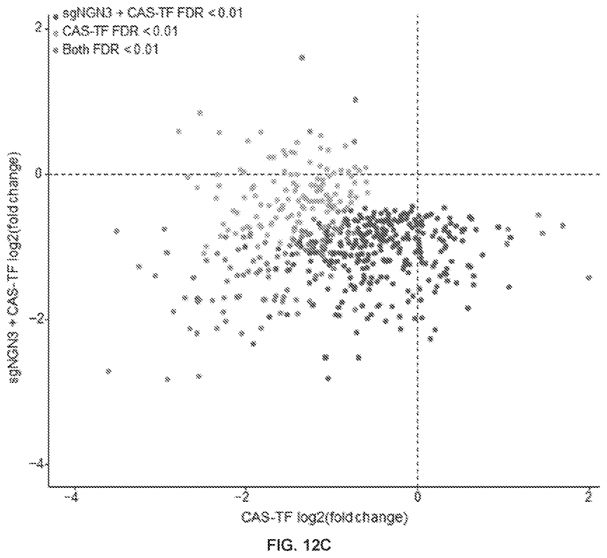

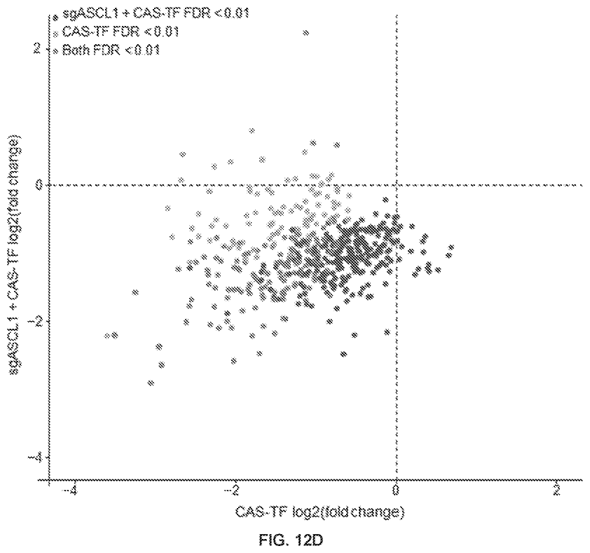

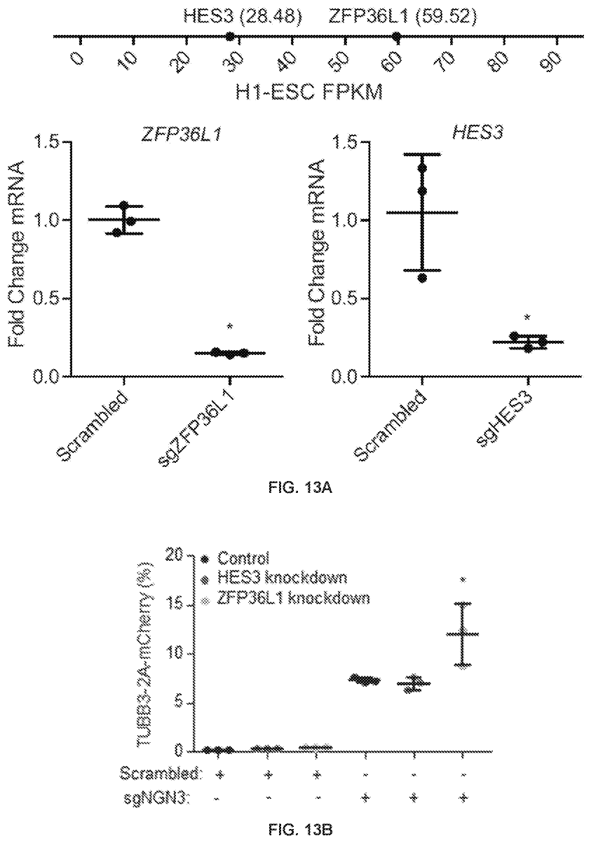

Effective cofactors that enhance conversion efficiency are often shared across different neuronal reprogramming paradigms but can contribute to subtype specification in context-dependent ways. Similarly, we hypothesized that many cofactors would be shared between NEUROG3 and ASCL1. Consistent with this hypothesis, we found that the majority of positive regulators were shared between the two screens (FIG. 3C). However, there were several factors enriched uniquely when combined with either NEUROG3 or ASCL1 (FIG. 3C). For example, FEV was positively enriched with NEUROG3 only, whereas NKX2.2 was positively enriched with ASCL1 only. Importantly, both the sgNGN3 and sgASCL1 screens identified novel TFs that were not observed in the single-factor CAS-TF screen (FIG. 12A-FIG. 12D). Many of these TFs, including LHX6, LHX8 and HMX2 are implicated in neuronal development and subtype specification, but have not been extensively characterized for the in vitro generation of neurons. A list of all candidate neurogenic factors identified across all three screens can be found in TABLE 1.

TABLE 1
All positive hits across the three neuronal differentiation screens.

| | sgNGN3 + | sgASCL1 + |
|---|---|---|
| Single Factor CRa-TF | CRa-TF | CRa-TF |
| NEUROG3 | PRDM1 | RUNX3 |
| SOX4 | LHX6 | PRDM1 |
| SOX9 | NEUROG3 | KLF6 |
| KLF4 | PAX8 | PAX2 |
| NR5A1 | SOX3 | RFX3 |
| NEUROD1 | KLF4 | SOX10 |
| SOX17 | FLI1 | GATA1 |
| SMAD1 | FOXH1 | KLF5 |
| ATOH1 | FEV | KLF1 |
| INSM1 | SOX17 | ERF |
| NEUROG1 | FOS | LHX6 |
| SOX18 | INSM1 | PHOX2B |
| RFX4 | SOX2 | NANOG |
| KLF7 | WT1 | NR5A2 |
| SP8 | SOX18 | ETV3 |
| OVOL1 | ZNF670 | NEUROG3 |
| NEUROG2 | LHX8 | SOX4 |
| ERF (from sublibrary) | OVOL1 | SOX9 |
| PRDM1 (from sublibrary) | E2F7 | PAX8 |
| OLIG3 (from sublibrary) | AFF1 | IRF5 |
| HIC1 (from sublibrary) | HMX2 | CDX4 |
| SOX3 (from sublibrary) | MAZ | RARA |
| FOXJ1 (from sublibrary) | RARA | BHLHE40 |
| SOX10 (from sublibrary) | PROP1 | SOX3 |
| KLF6 (from sublibrary) | FOSL1 | KLF4 |
| ASCL1 (from sublibrary) | PAX5 | NR5A1 |
| PLAGL2 (from sublibrary) | KLF3 | IRF4 |
| | | ASCL1 |
| | | GATA6 |
| | | SPIB |
| | | THRB |
| | | FOXH1 |
| | | NEUROD1 |
| | | SOX17 |
| | | CDX2 |
| | | ZEB2 |
| | | RARG |
| | | INSM1 |
| | | FOSL1 |
| | | NEUROG1 |
| | | SOX1 |
| | | WT1 |
| | | PAX5 |
| | | SOX18 |
| | | POU5F1 |
| | | RFX4 |
| | | KLF7 |
| | | NKX2-2 |
| | | OVOL2 |
| | | FOXJ1 |
| | | PRDM14 |
| | | VENTX |
| | | LHX8 |
| | | GFI1 |
| | | KLF17 |
| | | OVOL1 |
| | | OLIG3 |
| | | HMX3 |
| | | ZNF521 |
| | | ONECUT3 |
| | | OVOL3 |
| | | ZNF362 |
| | | AFF1 |
| | | HMX2 |
| | | ZNF786 |
| | | GATA5 |
| | | TBX3 |
| | | ZNF385A |
| | | ATOH1 |
| | | PROP1 |
| | | SOX11 |
| | | JUN |
| | | FOXE3 |
| | | FERD3L |
| | | E2F7 |

The positive hits from the two paired CAS-TF screens encompassed a diverse set of TF families (FIG. 3D). The majority of these TFs were not expressed or lowly expressed in pluripotent stem cells, however several factors were more highly expressed (Consortium, Nature 2012, 489, 57-74) (FIG. 3D). A set of eight TFs were chosen for further validations. These TFs were predicted to have minimal activity on their own, while enhancing the neurogenic activity when co-expressed with NEUROG3 and/or ASCL1 (FIG. 3E), While this subset of eight TFs was selected for further characterization, there are numerous other candidate factors revealed by the CRISPRa paired screens that could be subject to future studies (TABLE 1),

All of the TFs tested improved the conversion efficiency to mCherry-positive cells up to 3-fold when paired with sgNGN3 compared to sgNGN3 co-expressed with a scrambled gRNA (FIG. 3F). Because sgASCL1 only increased the mCherry reporter to modest levels, we chose to use NCAM staining for the gRNA validations for the pairings with this gRNA. Only E2F7 and HMX2 had modest effects on NCAM expression on their own (FIG. 3G). However, several of the TFs significantly increased the neurogenic activity of ASCL1, including up to 8-fold for E2F7 (FIG. 3G). Consistent with the predicted outcomes from the screens, NKX2.2 only had a significant effect with ASCL1, and not with NEUROG3 (FIG. 3E, FIG. 3F, and FIG. 3G),

### Example 6

### Neurogenic Transcription Factors Modulate Subtype Specificity and Maturation

Neuronal subtype identity and degree of synaptic maturation are important features defining the utility of in vitro-derived neurons for disease modeling and cell therapy applications. Consequently, the development of protocols to improve maturation kinetics and purity of subtype specification has been a primary focus in the field. Given the diversity of neurogenic TFs identified through our CRISPRa screens, and the range of conversion efficiencies observed through validation experiments, we reasoned that many of these TFs likely influence subtype identity and maturation in distinct ways. To begin to address this question, we performed bulk mRNA-sequencing to more globally assess the degree of neuronal conversion and compare the transcriptional diversity in neuronal populations generated with different TFs.

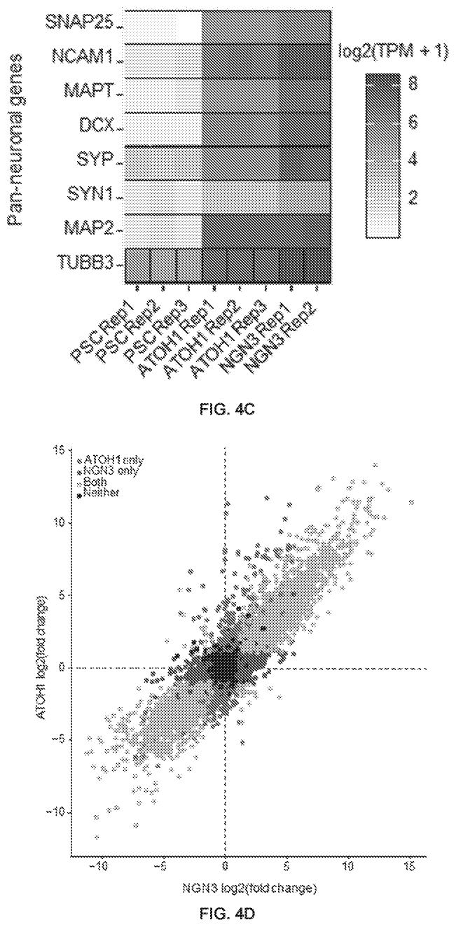

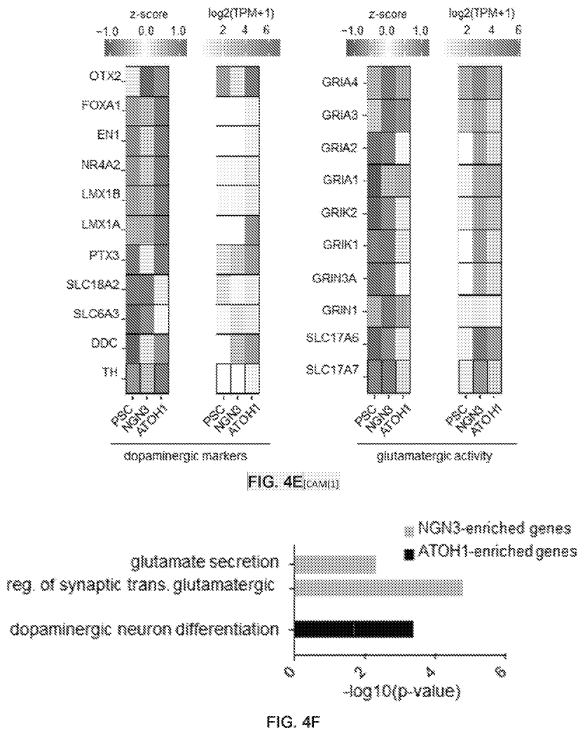

We started by analyzing neurons derived from a single TF. While combinations of TFs often enhance the specificity of subtype generation and improve the conversion efficiency and maturation kinetics, single TFs can be sufficient to generate functional neurons with subtype proclivity. We chose to first perform mRNA-sequencing on neurons derived from either ATOH1 or NEUROG3 overexpression (FIG. 4A-FIG. 4F). These TFs had some of the highest conversion efficiencies determined through validation experiments (FIG. 2A-FIG. 2F), which facilitates the isolation of sufficient material for sequencing. Additionally, while the neurogenic activity of both ATOH1 and NEUROG3 has been confirmed previously, our understanding of the role of ATOH1 and NEUROG3 in in vitro neuronal differentiation remains incomplete.

We overexpressed the cDNAs encoding either ATOH1 or NEUROG3, used FACS to purify TUBB3-mCherry-positive cells and performed mRNA-sequencing after seven days of transgene expression. Both populations of neurons had over 3000 genes up-regulated relative to the starting population of undifferentiated pluripotent stem cells (FIG. 4A). The set of shared genes was enriched in gene ontology (GO) terms associated with neuronal differentiation and development (FIG. 4B). Importantly, a set of pan-neuronal genes was highly enriched across all replicates for ATOH1 (3 replicates) and NEUROG3 (2 replicates) relative to pluripotent stem cells (FIG. 4C).

Surprisingly, we observed a strong correlation across all detectable genes between ATOH1 and NEUROG3-derived neurons, indicating a striking consistency in the induction of the core neuronal program and suppression of the pluripotency network (FIG. 4D). However, a subset of genes was more highly expressed with either ATOH1 or NEUROG3 (FIG. 4D). These genes were enriched in GO terms related to glutamatergic activity for NEUROG3 and dopaminergic activity for ATOH1 (FIG. 4E). Indeed, when we examined a set of markers expected of the two neuronal subtypes, we found clear enrichment in dopaminergic markers for ATOH1 and glutamatergic markers for NEUROG3 (FIG. 4F). While certain canonical markers of dopaminergic neurons, such as tyrosine hydroxylase (TH), remained lowly expressed, many TFs associated with dopaminergic specification, such as LMX1A, were more highly expressed in ATOH1-derived neurons (FIG. 4F).

In many cases, combinations of TFs can aid in the precision of neuronal subtype specification or enhance conversion efficiency and maturation. We reasoned that the cofactors identified in our paired gRNA screens would serve as prime candidates for modulating subtype identity and maturation when combined with neurogenic factors identified in the single-factor screen. Consequently, we chose to perform mRNA-sequencing on neurons derived from NEUROG3 alone or in combination with either E2F7, RUNX3, or LHX8. These three cofactors were preferentially selected due to their substantial influence on differentiation efficiency assessed through gRNA validations (FIG. 3A-FIG. 3G). We chose NEUROG3 due to its defined preference for generating glutamatergic neurons, often considered a default subtype. We overexpressed the cDNAs encoding NEUROG3 alone or in combination with E2F7, RUNX3, or LHX8 and performed mRNA-sequencing after six days of transgene expression.

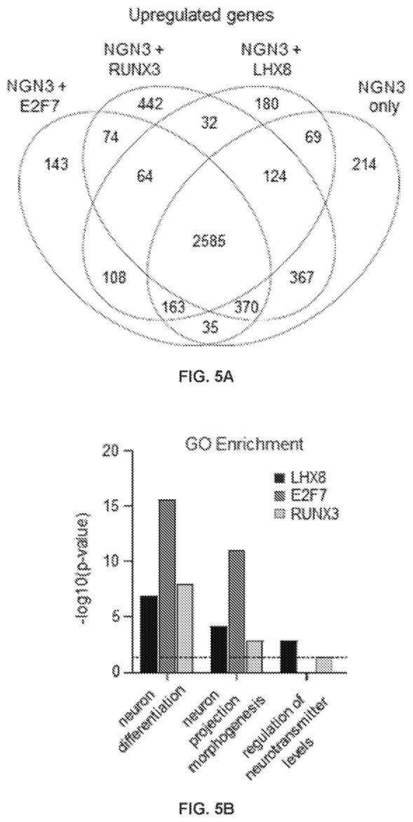

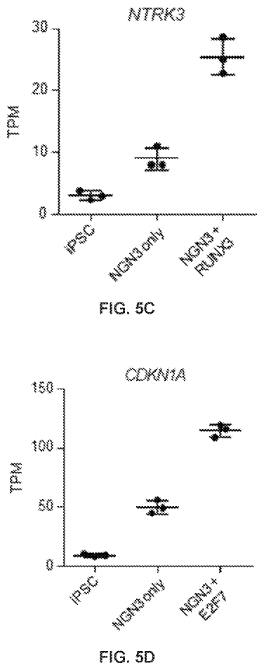

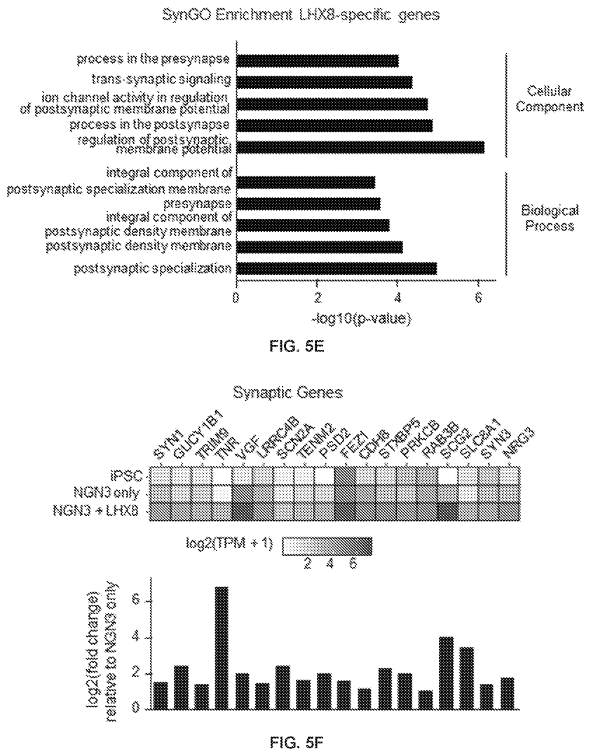

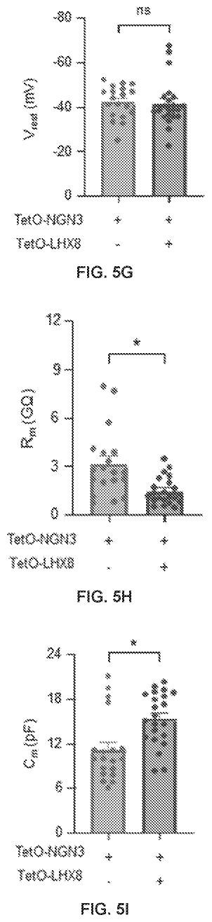

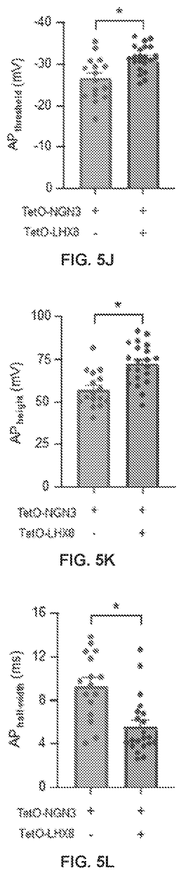

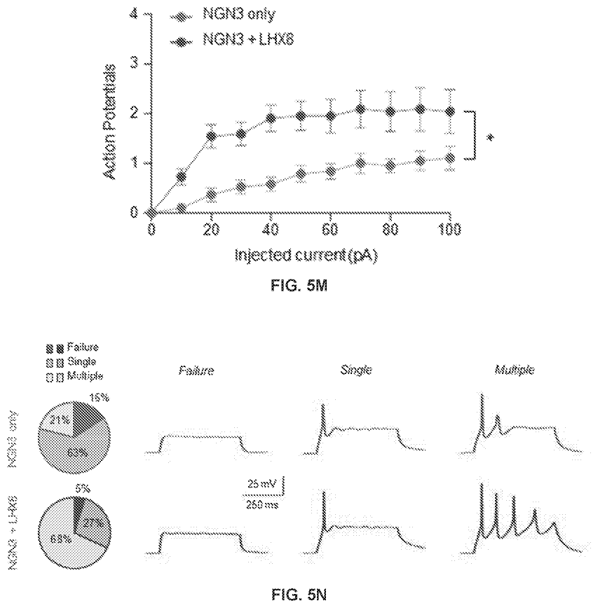

Similar to the ATOH1 and NEUROG3 comparison, all TF pairs shared a core set of up-regulated genes (FIG. 5A). However, genes uniquely up-regulated with each TF pair relative to NEUROG3 alone were enriched in GO terms related to neuronal differentiation and development, consistent with the previously measured increase in TUBB3 expression and improvements in conversion efficiency with expression of these neuronal cofactors (FIG. 5B).

Importantly, each TF pair uniquely up-regulated genes related to specification and maturation of particular neuronal subtypes. For example, the addition of RUNX3 led to an increase in expression of NTRK3, encoding the TrkC neutrophin-3 receptor linked to the development of proprioceptive dorsal root ganglion neurons (FIG. 5C). The addition of E2F7 led to an increase in CDKN1A, encoding the p21 cell cycle regulator involved in neuronal late commitment and morphogenesis (FIG. 5D). A subset of genes more highly expressed with the addition of LHX8 were enriched in synaptic gene ontology (SynGO) terms associated with synaptic development, a hallmark of neuronal maturation (FIG. 5E). In agreement with the GO term analysis, a set of genes related to synapse development, regulation and function were clearly up-regulated with the addition of LHX8 (FIG. 5F).

To evaluate if the addition of LHX8 influenced the electrophysiological maturation of NEUROG3-derived neurons, we performed patch-clamp recordings of TUBB3-2A-mCherry-positive cells seven days after transgene induction, While we did not observe a difference in the resting membrane potential (FIG. 5G), we did observe a decrease in membrane resistance (FIG. 5H) and an increase in membrane capacitance (FIG. 5I) with the addition of LHX8 relative to NEUROG3 alone. Several metrics of action potential maturation were improved with LHX8, including a decrease in firing threshold (FIG. 5J), an increase in action potential height (FIG. 5K) and a decrease in action potential half-width (FIG. 5L). Additionally, neurons with LHX8 fired action potentials at higher frequency for a given step depolarization with current injection (FIG. 5M) and had a higher proportion of recorded cells that fired multiple actions potentials (FIG. 5N). Cells generated with NEUROG3 alone more frequently failed to fire or only fired a single low-amplitude action potential (FIG. 5N).

### Example 7

### Combinatorial gRNA Screens Identify Negative Regulators of Neuronal Fate

The conversion efficiencies achieved with cell reprogramming and differentiation protocols often vary depending on the starting and ending cell types. Generally, more distantly related cell types, or more aged cell lines, are less amenable to conversion. For instance, the reprogramming of astrocytes to neurons is often more efficient than that of fibroblasts to neurons, with efficiencies further reduced in adult fibroblasts relative to embryonic fibroblasts. These discrepancies in reprogramming outcomes can in part be explained by variation in gene expression profiles and epigenetic landscapes of cells of different type or developmental age. Consequently, this cellular context can create a barrier preventing proper TF activity, reducing conversion efficiency and fidelity.

High-throughput loss-of-function RNAi screens have been instrumental in the identification of molecular barriers preventing cell type reprogramming and influencing conversion efficiencies. Importantly, ablation of such barriers often results in significant improvements in reprogramming outcomes. Through our paired CRISPRa screens, we identified TFs whose activation impeded neuronal differentiation (FIG. 3B and FIG. 11A and FIG. 11B). These candidate negative regulators included several members of the HES gene family of canonical neuronal repressors downstream of Notch signaling, in addition to many other uncharacterized TFs. A list of all candidate negative regulators identified across all three screens can be found in TABLE 2.

TABLE 2
Al! negative hits across the three neuronal differentiation screens.

| | sgNGN3 + | sgASCL1 + |
|---|---|---|
| Single Factor CRa-TF | CRa-TF | CRa-TF |
| ZIC2 | HES2 | ETV1 |
| SPI1 | SREBF1 | ZIC2 |
| GRHL2 | CIC | GSC2 |
| TFAP2C | WHSC1 | CIC |
| KLF8 | VDR | GRHL2 |
| MYB | HES1 | REST |
| TCF21 | ID2 | TFAP2C |
| KLF12 | TCF21 | SALL1 |
| TWIST1 | SNAI1 | NFKB1 |
| SNAI1 | RREB1 | ELF2 |
| RREB1 | GCM2 | HES1 |
| GCM2 | IRF3 | MYB |
| GRHL1 | FOXA1 | KLF12 |
| ETS1 | GATA5 | VSX2 |
| BARHL2 | GRHL1 | NFE2 |
| GRHL3 | SOX5 | SNAI1 |
| ELF3 | DMRT1 | TRERF1 |
| PTF1A | GCM1 | RREB1 |
| GSX1 | BARHL2 | IRF1 |
| PBX2 | SOX13 | IRF3 |
| NOTO | ZEB1 | KLF2 |
| KLF3 | PITX2 | MYOD1 |
| ZNF311 | PTF1A | SOX15 |
| ELMSAN1 | ZNF282 | BARX1 |
| ZNF296 | NPAS2 | GRHL1 |
| PLEK | ZNF160 | SOX5 |
| KMT2A | HES7 | ETS1 |
| HES3 | ZBED4 | SKIL |
| | SALL4 | BARHL2 |
| | GLIS3 | SOX13 |
| | TBX22 | ERG |
| | ZNF331 | GRHL3 |
| | EGR4 | ZNF281 |
| | ZIC5 | ELF3 |
| | ZNF710 | HESX1 |
| | ZNF697 | KLF15 |
| | ZFP36L2 | PITX2 |
| | ELMSAN1 | PTF1A |
| | ZNF296 | GSX1 |
| | ZNF318 | ZNF160 |
| | ZNF570 | ETV5 |
| | ZNF683 | MYBL1 |
| | ZFP36L1 | NOTO |
| | HES4 | DPF1 |
| | ZNF777 | MECOM |
| | HESS | GLIS3 |
| | ZIM2 | KLF3 |
| | ZNF579 | TBX22 |
| | BMP2 | ESX1 |
| | CRAMP1L | ZNF337 |
| | TOX3 | ZFP36L2 |
| | FEZF2 | ELMSAN1 |
| | HES3 | ZNF618 |
| | ZNF791 | ZNF296 |
| | | ZNF318 |
| | | ZNF570 |
| | | ZNF497 |
| | | ZFP36L1 |
| | | HES5 |
| | | BMP2 |
| | | CRAMP1L |
| | | ZNF821 |
| | | KMT2A |
| | | HES3 |
| | | BSX |

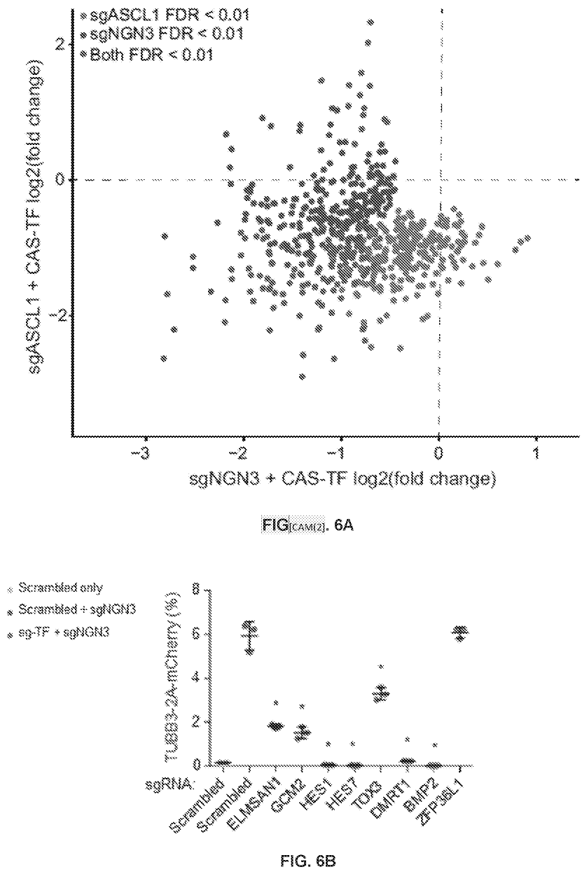

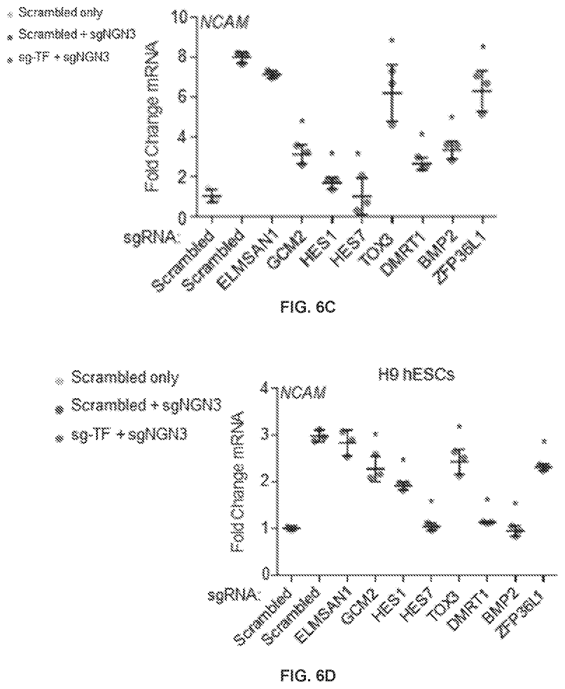

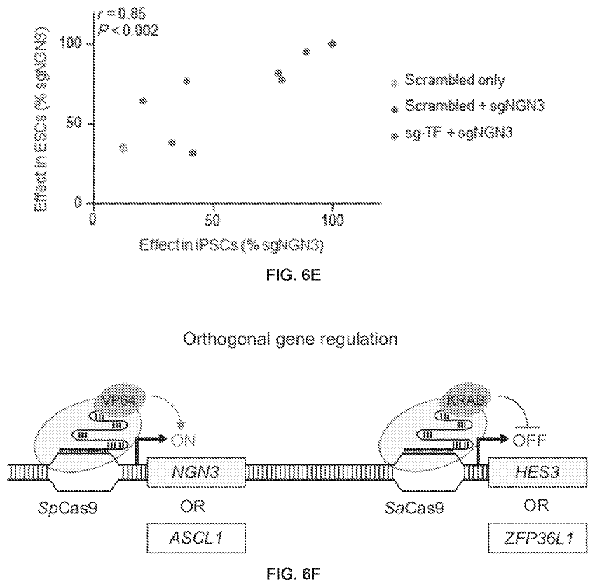

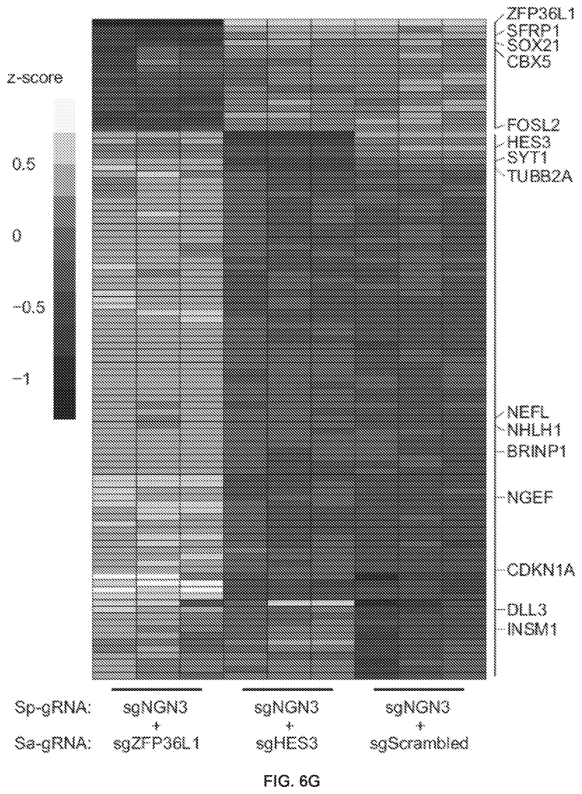

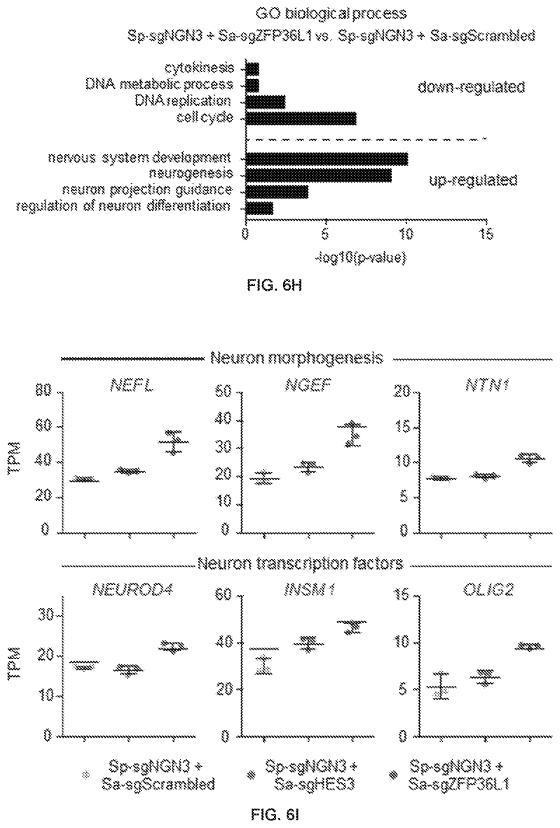

Interestingly, the majority of the negative regulators were shared across the sgNGN3 and sgASCL1 screens (FIG. 6A). They consisted of a diverse set of TFs across many TF families with a wide range of basal expression in embryonic stem cells, When tested individually with single gRNAs co-expressed with a NEUROG3 gRNA, several of the TFs, including HES1 and DMRT1, reduced the percent of rnCherry-positive cells back to basal levels (FIG. 6B), To prove that this repression was not confined to only the reporter gene, we also demonstrated reductions in NCAM expression up to 8-fold with seven of the eight repressive factors tested (FIG. 6C). We similarly observed repression of neuronal differentiation when these factors were tested in H9 human embryonic stem cells (FIG. 6D). In fact, there was a striking correlation between the relative influence of these negative regulators in iPSCs versus ESCs (FIG. 6E), underscoring the robustness of these effects across multiple pluripotent stern cell lines.

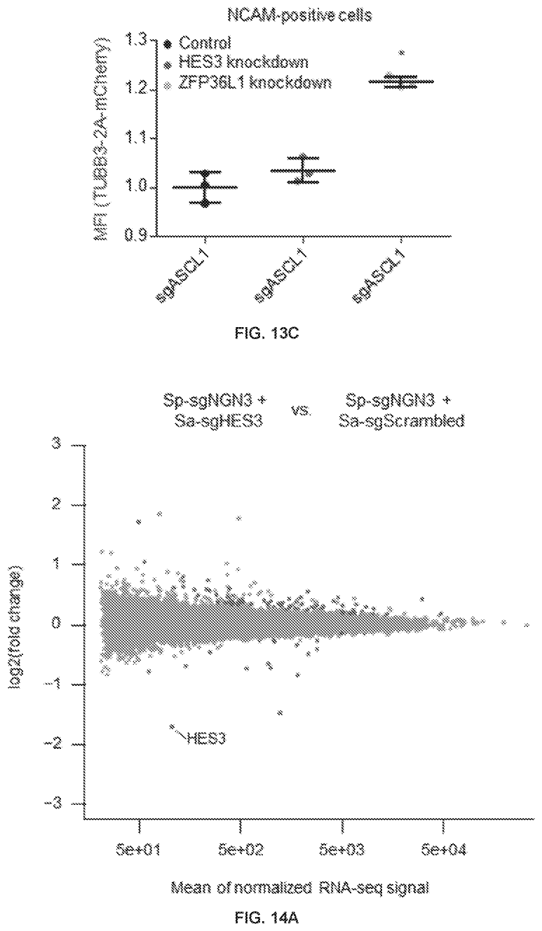

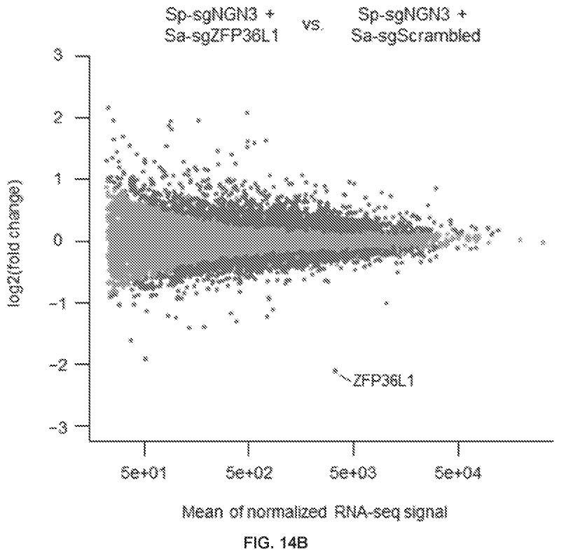

We reasoned that some of these identified negative regulators that were expressed basally in pluripotent stern cells may serve as barriers to neuronal conversion, and that their inhibition could improve differentiation efficiency. Cas9 proteins from different bacterial species can be programmed for orthogonal gene regulation and epigenetic modification. Therefore, we chose to use the orthogonal dSaCas9^{KRAB} (Thakore et al, Nat. Commun. 2018, 9, 1674), based on the Cas9 protein from S. aureus, to target the promoters of two negative regulators expressed basally in pluripotent stem cells, ZFP36L1 and HES3 (FIG. 6F). Targeting the promoters of these genes with dSaCas9^{KRAB} led to transcriptional repression of 10-fold and 4-fold for ZFP36L1 and HES3, respectively (FIG. 13A).

The use of dSaCas9^{KRAB} for targeted gene repression enables the co-expression of the orthogonal ^{VP64}dSpCas9^{VP64} for concurrent activation of a neurogenic factor (FIG. 6F). TUBB3-2A-mCherry ^{VP64}dSpCas9^{VP64}iPSCs were first transduced with a dSaCas9^{KRAB} lentivirus that co-expresses a ZFP36L1, HES3, or scrambled S. aureus gRNA. After nine days post-transduction of the S. aureus gRNAs, cells were transduced with a lentivirus encoding either sgNGN3 or sgASCL1 from S. pyogenes and analyzed four days after this final transduction. Knockdown of ZFP36L1 increased the percent mCherry-positive cells obtained with sgNGN3 2-fold relative to a control cell line expressing a scrambled S. aureus gRNA (FIG. 13B). Similarly, ZFP36L1 knockdown increased the mCherry reporter gene expression level 1.2-fold in the NCAM-positive population of differentiating cells obtained with sgASCL1 (FIG. 13C).

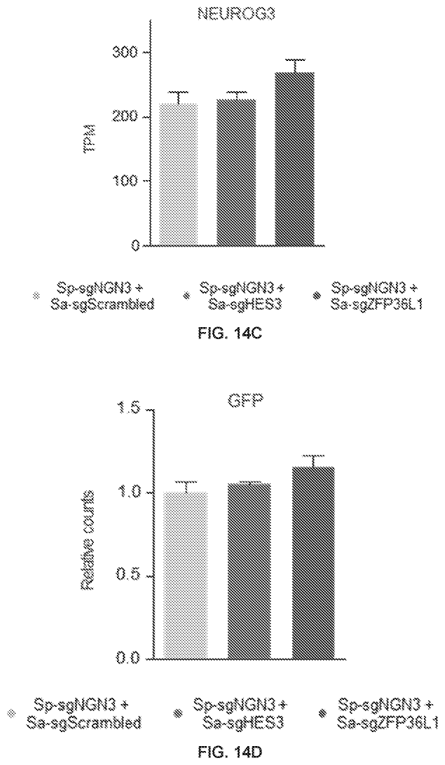

To identify the genome-wide effects of this orthogonal CRISPR-based regulation, we performed mRNA-sequencing on neurons derived from NGN3 activation concurrent with repression of ZFP36L1 or HES3. While knockdown of HES3 resulted in only a few subtle changes in gene expression relative to cells that received a scrambled S. aureus gRNA (FIG. 14A), knockdown of ZFP36L1 led to a significant change in the global gene expression profile (FIG. 6G and FIG. 14B) relative to activation of NGN3 alone. We did also observe a subtle increase in expression of NEUROG3 and of the S. pyogenes gRNA, quantified by expression of a GFP transgene on the gRNA vector, in ZFP36L1 knockdown cells (FIG. 14C and FIG. 14D). Genes up-regulated in neuronal cells with ZFP36L1 knockdown were enriched in GO terms related to neuronal differentiation and morphological development (FIG. 6H). In contrast, genes down-regulated with ZFP36L1 knockdown were enriched in GO terms related to cell cycle development and progression (FIG. 6H), Examples of genes up-regulated with ZFP36L1 knockdown include the neuronal transcription factors NEUROD4, INSM1, and OLIG2, as well as genes involved in neuronal morphogenesis, including NEFL, NGEF, and NTN1 (FIG. 6I).

### Example 8

### Discussion

As detailed herein, we systematically profiled 1,496 putative human transcription factors for their role in regulating neuronal differentiation of pluripotent stem cells through single and combinatorial CRISPRa screens. This work underscores the utility of CRISPR-based technologies for perturbing gene expression in a high-throughput manner and highlights the robust nature of dCas9-based gene activation for studying the causal role of gene expression in complex cellular phenotypes.

The use of an early pan-neuronal marker like TUBB3 as a proxy for a neuronal phenotype enabled the identification of a broad set of TFs with varying neurogenic activity. For instance, while NEUROG3 was sufficient to rapidly generate neuronal cells within four days of expression, ATOH7 and ASCL1 required more extended time in culture to achieve a similar phenotype (FIG. 2D and FIG. 2E), It is likely that the addition of cofactors, like those identified in our combinatorial gRNA screens, could improve the efficiency and kinetics of differentiation as seen with other cell reprogramming studies (Pang et al. Nature 2011, 476, 220-223). Additionally, several TFs, including KLF7, NR5A1 and OVOL1, induced the expression of TUBB3 but failed to generate neuronal cells (FIG. 2D), These TFs might serve as cofactors or downstream regulators that require the co-expression of other neurogenic factors to obtain a more complete differentiation. Indeed, many of the TFs identified in the single-factor screen were also hits in the paired gRNA screens (TABLE 1).

We found that several IFs with clear neurogenic activity, including ASCL1 and ATOH7, had only a single gRNA enriched in the CAS-TF screen (FIG. 8). Because a single enriched gRNA could be the result of off-target activity or noise, it may be challenging to accurately classify these gRNAs. The use of more gRNAs per gene or next-generation dCas9-based activator platforms might help to more accurately define true positive effects. Indeed, our sub-library screen with a greater number of gRNAs per gene revealed several additional candidate hits (FIG. 10). Further improvements in gRNA design and screen analysis may continue to make CRISPR-based screens more robust and extensible to more complex phenotypes.

Through the use of paired gRNA screens, we identified a set of TFs that improved neuronal differentiation efficiency, maturation, and subtype specification. Interestingly, the majority of these TFs did not possess neurogenic activity on their own, as assessed in our single-factor CAS-TF screen. This observation underscores the importance of synergistic TF interactions that govern cell differentiation and supports the use of unbiased methods to identify these TFs. We identified E2F7 as improving neuronal conversion efficiency (FIG. 3F and FIG. 3G), possibly due to its known role in inhibiting cell proliferation, an important switch in the conversion from proliferative pluripotent stern cell to post-mitotic neuron. Additionally, we found that RUNX3 uniquely induced subtype-specific receptor gene expression (FIG. 5C), and thus could be a useful addition to differentiation protocols to more precisely guide neuronal subtype identity. The neuronal cofactor LHX8 had a profound influence on markers of neuronal maturation, as seen with enrichments of many synapse-related genes and clear improvements in electrophysiological maturation (FIG. 5). Functional synapse formation is an essential phenotype for in vitro-derived neurons, and it is often the rate-limiting step. Improving synaptic maturation through TF programming could serve to expedite the development of useful neuronal models for disease modeling and drug screening.

Future studies may take advantage of advanced screening platforms to further characterize cell lineage specifying factors. A more comprehensive list of neuronal TFs may have been identified by performing screens that relied on multiple neuronal markers, or that used markers of maturation or subtype identity. Alternatively, rather than assaying for a few discrete markers, these screens could be performed with a single-cell RNA-sequencing (scRNA-seq) output to more accurately define the diversity of neuronal phenotypes obtained with different TF combinations and benchmark these results against the growing atlas of scRNA-seq data from human brain samples. The TFs identified from the screens detailed herein may serve as prime candidates lor sub-libraries to test in these alternative approaches that may be more limited in the scale of library size.

The paired gRNA screens also identified negative regulators of neuronal differentiation. Knockdown of one of those TFs, ZFP36L1, was sufficient to improve differentiation, leading to global changes in gene expression towards a more differentiated neuronal phenotype (FIG. 6G, FIG. 6H, FIG. 6I). While the effects on differentiation were somewhat modest in this example, more dramatic improvements might be seen in cell types that are less amenable to conversion, such as adult aged fibroblasts. Importantly, many of the negative regulators identified in our screens are expressed in other cell types used for reprogramming studies, such as fibroblasts and astrocytes.

Additional CRISPRa screens targeting epigenetic modifiers or other gene subsets besides TFs may help further elucidate the extent to which gene activation can modulate neuronal cell fate. The continued development of synthetic systems for programmable regulation of endogenous gene expression and chromatin state, and the application of these systems to more complex in vitro and in vivo models, may enable studies to more comprehensively define the gene networks and epigenetic mechanisms that govern cell fate decisions,

Overall, as detailed herein, we have identified a broad set of transcription factors that control neuronal fate specification in human cells. This catalog of factors may serve as a basis for the development of protocols for the generation of diverse neuronal cell types at high efficiency and fidelity for applications in regenerative medicine and disease modeling. Ultimately, the CRISPRa screening platform detailed herein may be extended to other cell reprogramming paradigms and facilitate the in vitro production of many clinically relevant cell types.

### Example 9

### High-Throughput CRISPR Activation Screen to Identify Novel Drivers of Myogenic Progenitor Cell Fate

Skeletal muscle regeneration is a complex process mediated by the muscle satellite cells. The cascade of events that drive proper myogenic differentiation from muscle satellite cells is well characterized; however, the upstream events that specify satellite cell fate during embryonic development are not as thoroughly understood. The transcription factor, PAX7 plays a pertinent role in specification and maintenance of satellite cells and its overexpression can specify rnyogenic progenitor cell fate in human pluripotent stem cells. To investigate novel drivers of satellite cell fate, we generated a PAX7-2a-GFP cell line in human H9 embryonic stem cells. We applied a gRNA library targeted at the promoter of all human transcription factors and co-delivered a CRISPR/Cas9-based transcriptional activator to systematically identify independent drivers of PAX7 expression. We then performed a second screen to investigate co-factors of PAX7 by applying the gRNA library along with a PAX7 promoter-targeting gRNA. This second screen identified a separate set of transcription factors, and together, a total of 21 transcription factors were identified. Individual validations demonstrated induction of PAX7 expression and adoption of a myogenic cell fate for some of the hits. The data generated from this study can be used for potential therapeutic targets for skeletal muscle regeneration in the context of cell and gene therapies.

Generation of a PAX7-2a-GFP Cell Line. Human H9 ESCs (obtained from the WCell Stem Cell Bank) were used for these studies and were maintained in mTeSR (Stem Cell Technologies) and plated on tissue culture treated plates coated with ES-qualified Matrigel (Corning). H9 ESCs were co-transfected with a Cas9-gRNA plasmid targeting the PAX7 isoform A stop codon and a donor plasmid with homology arms complementary to exon 8 and the 3′UTR of PAX7 isoform A. Transfections were performed with a GenePulser Xcell (Bio-Rad) at 250 V, 750 pF, and infinite resistance in a 4mm cuvette. The donor plasmid also contained a PGK-PuroR cassette surrounded by IoxP sites to allow for selective expansion of cells with donor plasmid integration. After two weeks of puromycin selection (1 μg/mL), clones were picked and screened by PCR for integration of the donor cassette at the correct genomic locus. Select positive clones were transfected with a Cre recombinase plasmid to remove the large PGK-PuroR cassette. Cells were plated sparsely and clones were picked and screened for correct integration using primers outside the donor template. Resulting PCR bands were confirmed by Sanger sequencing.

Generation of CRISPR Activation-Transcription Factor (CRa-TF) gRNA Library. Putative human transcription factors were selected based off of a previously curated list. The corresponding gRNAs available for the list of genes were extracted from the human subpooled CRISPRa library. The 100 scrambled non-targeting gRNAs were also extracted from this library. Our custom library consists of 5 gRNAs targeted per transcriptional start site for 1496 unique genes and the 100 scrambled non-targeting gRNAs for a total library size of 8,505 gRNAs. The oligonucleotide pool (Custom Array) was PCR amplified and cloned using Gibson assembly into the single gRNA expression plasmid for the single CRa-TF screen or the dual gRNA expression plasmid for the paired CRa-TF screens with a PAX7 promoter targeting gRNA.

Lentivirus Production. HEK293T cells were obtained from the American Tissue Collection Center (ATCC) and purchased through the Duke University Cancer Center Facilities and were cultured in Dulbecco's Modified Eagle's Medium (Invitrogen) supplemented with 10% FBS (Sigma) and 1% penicillin/streptomycin (Invitrogen) at 37° C. with 5% CO2. Approximately 3.5 million cells were plated per 10 cm TCPS dish. Twenty-four hours later, the cells were transfected using the calcium phosphate precipitation method with the expression plasmid, pMD2.G enveloping plasmid (Addgene #12259), and psPAX2 second-generation packaging plasmid (Addgene #12260). The medium was exchanged 12 hours post-transfection, and the viral supernatant was harvested 24 and 48 hours after this medium change. The viral supernatant was pooled and centrifuged at 500 g for 5 minutes, passed through a 0.45 μm filter, and concentrated to 20× using Lenti-X Concentrator (Clontech) in accordance with the manufacturer's protocol. Lentiviral gRNA libraries were titered by flow cytometry.

High-Throughput CRa-TF Screen for Upstream Regulators of PAX7. Undifferentiated H9 PAX7-2a-GFP cells stably expressing ^{VP64}dCas9^{VP64} were dissociated and 22.5×10⁶ cells were transduced (3.1×10⁴ cells/cm²) with the CRa-TF lentiviral library at an MOI of 0.2 per replicate. We aimed to achieve 500-fold coverage of the library per replicate. Cells were selected with 1 μg/mL of puromycin for 6 days. For differentiation, the hESCs were dissociated into single cells with Accutase (Stem Cell Technologies) and plated on Matrigel-coated plates (3.6×10⁴ cells/cm²) in in mTeSR medium supplemented with 10 μM Y27632 (Stem Cell Technologies). The following day, mTeSR medium was replaced with E6 media supplemented with 10 μM CHIR99021 (Sigma) to initiate mesoderm differentiation. After 2 days, CHIR99021 was removed and cells were maintained in E6 media with 10 ng/mL FGF2 (Sigma) supplemented daily. Cells were unpassaged during the duration of differentiated for 2 weeks in version 1 of the screen and for 1 week in version 2 of the screen before analysis.

At 1 or 2 weeks after induction of differentiation, cells were dissociated with 0.2% Collagenase II (ThermoFisher) and washed with neutralizing media (10% FBS in DMEM/F12). Cells were pelleted by centrifugation and resuspended in flow media (5% FBS in PBS). Cells were gated for positive mCherry expression and the top 10% and bottom 10% of GFP expressing cells were sorted on the SONY SH800 flow cytometer into separate tubes. Sorted cells were pelleted and genomic DNA was extracted using the Qiagen DNeasy kit. Unsorted cells were also set aside for genomic DNA isolation to serve as an input control.

The gRNA sequences were recovered from the genomic DNA by PCR. Sequencing was performed on an Illumina Miseq with 21bp paired-end sequencing using custom read and index primers.

Data Processing and Enrichment Analysis. FASTQ files were aligned to custom indexes (generated from the bowtie2-build function) using Bowtie with the options -p 32—end-to-end—very-sensitive −3 1-I 0-X 200. Counts for each gRNA were extracted and used for further analysis. All enrichment analysis was performed using R. For individual gRNA enrichment analysis, the DESeq2 package was used to compare between high and low, unsorted and low, or unsorted and high conditions for each screen.

Individual gRNA Validations. The protospacers from the top enriched gRNAs found in each screen were order as oligonucleotides from IDT and cloned into a lentiviral gRNA expression vector as described earlier. The same H9 PAX7-2a-GFP cell line used in the pooled CRa-TF screen were used for the individual gRNA validations. The cells were transduced with individual gRNAs and underwent the same purornycin selection and differentiation protocol as in the original screens, but in a smaller scale.

RNA was isolated using the RNeasy Plus RNA isolation kit (Qiagen). cDNA was synthesized with the SuperScript VILO cDNA Synthesis Kit (Invitrogen). Real-time PCR using PerfeCTa SYBR Green FastMix (Quanta Biosciences) was performed with the CFX96 Real-Time PCR Detection System (Bio-Rad). The results are expressed as fold-increase expression of the gene of interest normalized to GAPDH expression using the ΔΔCₜ method.

Immunofluorescence Staining of Cultured Cells. For differentiation, cells were grown to confluency and differentiated on 24 well tissue culture plates coated with Matrigel, and immunofluorescence staining was performed directly in the well. Cells were fixed with 4% PFA for 15 min and permeabilized in blocking buffer (PBS supplemented with 3% BSA and 0.2% Triton X-100) for 1 hr at room temperature. Samples were incubated overnight at 4° C. with PAX7 (1:20, Developmental Studies Hybridoma Bank) and Myosin Heavy Chain MF20 (1:200, Developmental Studies Hybridoma Bank). Samples were washed with PBS for 15 min and incubated with compatible secondary antibodies diluted 1:500 from Invitrogen and DAPI for 1 hr at room temperature. Samples were washed for three times for 5 min with PBS and wells were kept in PBS and imaged using conventional fluorescence microscopy.

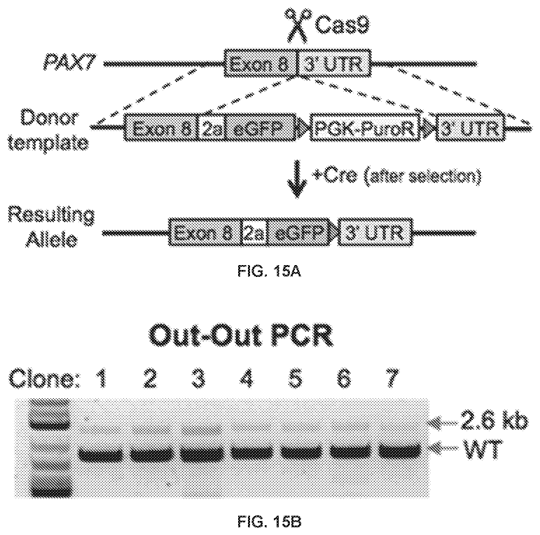

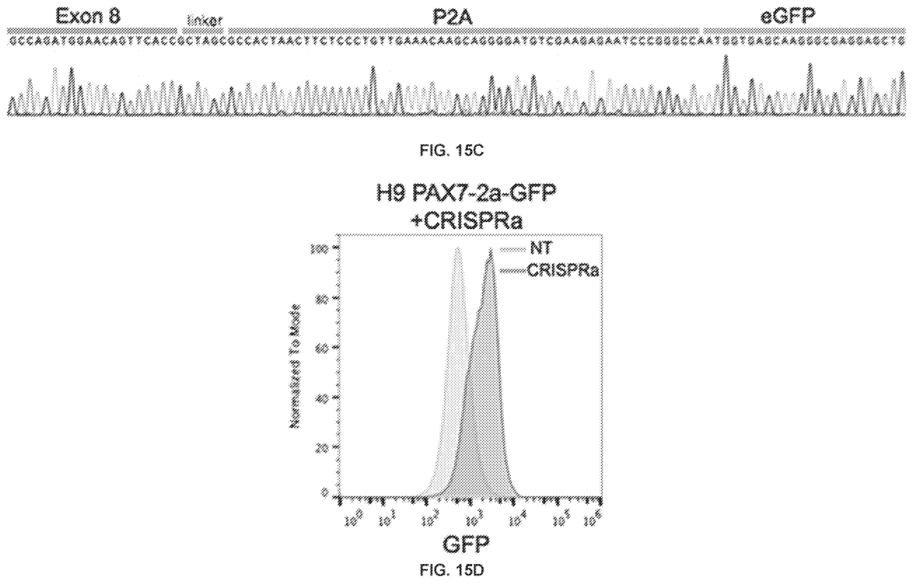

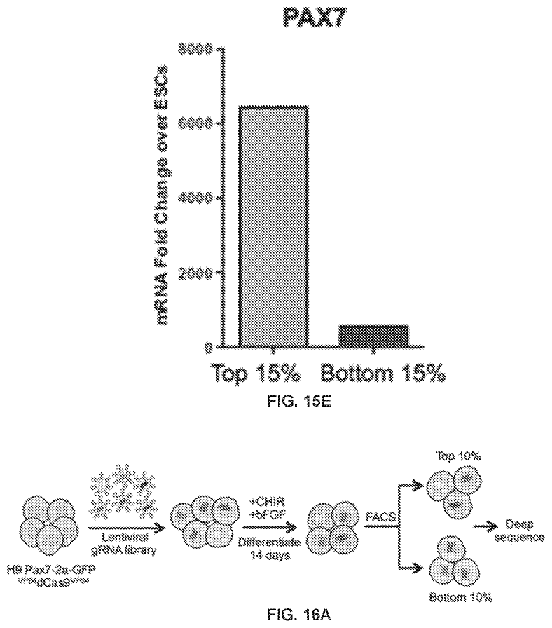

Results: Generation of PAX7 Reporter Line in Human ESCs. PAX7 may be critical for satellite cell specification, function, and maintenance. Because adult satellite cells are also identified by their unique expression of PAX7, we decided to use this gene to generate a satellite cell reporter line. We tested three gRNAs designed to cut near the stop codon of PAX7 in H9 ESCs and found highest cutting activity with gRNA 1 by SURVEYOR analysis. We designed a donor template that contained homology arms and a P2A-eGFP sequence to be inserted downstream of the last exon of PAX7 (FIG. 15A), H9 ESCs were co-transfected with CRISPR/Cas9 plasmids and the donor vector, which contains a loxP-flanked PGK-PuroR cassette to allow for selection of recombination events. Resistant clones were molecularly validated and the selection cassette was excised by Cre recombination. Resulting clones were further validated by PCR with primers designed to pan outside the homology arms (FIG. 15B). Larger integration bands of multiple clones were validated by Sanger sequencing to ensure in-frame positioning of the reporter cassette (FIG. 15C). The smaller wild-type band was also sequenced to ensure no indels were generated on the non-reporter allele. One clone was selected and used for subsequent studies.

Reporter activity was validated by transducing cells with a lentiviral vector encoding ^{VP64}dCas9^{VP64} and a gRNA targeted at the PAX7 promoter to activate endogenous gene expression. Flow cytometry analysis showed a clear shift in GFP expression in the clonal population compared to non-transduced cells (FIG. 15D). The top 15% and bottom 15% of GFP expressing cells were sorted, and RNA was extracted for qRT-PCR, which demonstrated positive correlation of GFP to PAX7 expression (FIG. 15E).

CRa-TF Screen to Identify Novel Regulators of PAX7 Expression. To systematically identify TFs that act upstream of PAX7, we generated a gRNA library targeting the promoter of all putative TFs, based off of a previously curated list. The corresponding gRNAs available for the list of genes were extracted from the human subpooled CRISPRa library previously generated. The custom CRISPRa-TF (CRa-TF) library generated for our studies included 5 gRNAs targeted per transcriptional start site for 1496 unique genes and 100 scrambled non-targeting gRNAs for a total library size of 8,505 gRNAs.

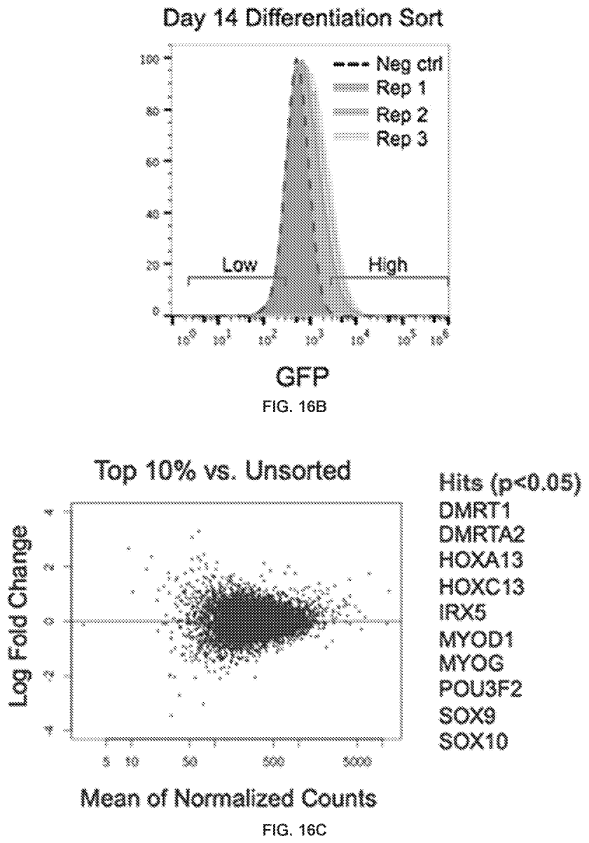

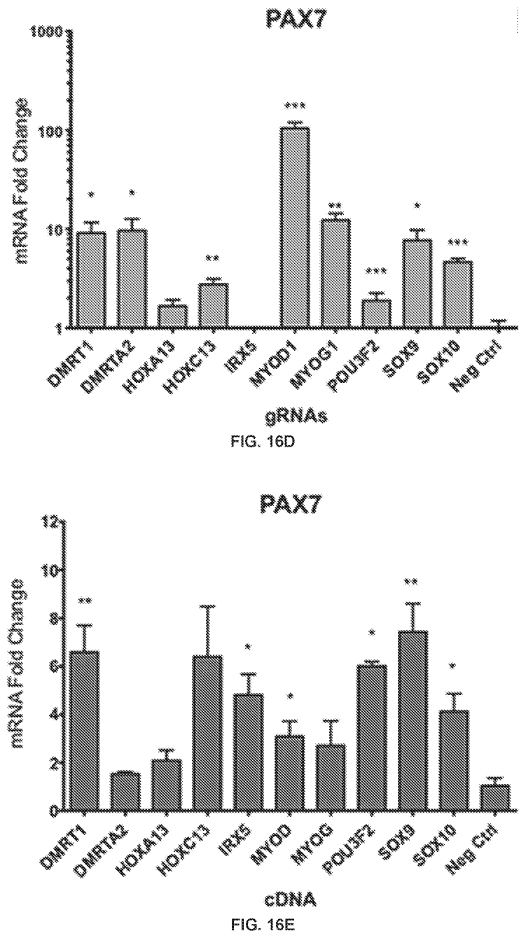

Because PAX7 is expressed in the ectoderm-derived neural crest during embryogenesis, we paired our screen with a mesoderm differentiation protocol to promote myogenic lineage specification. Differentiation of hPSCs into mesoderm cells can be initiated by addition of the small molecule CHIR99021, a GSK3 inhibitor. Prior to differentiation, we transduced our cell line to stably express ^{VP64}dCas9^{VP64}. We next transduced our CRa-TF library at an MOI of 0.2, applied selection, and allowed cells to differentiate for 2 weeks in the presence of FGF2 in serum-free media conditions (FIG. 16A). We had previously determined that 2 weeks of mesodermal differentiation alone is not sufficient to induce GFP expression.

With the CRa-TF library and differentiation, a discernable population of GFP+ cells emerged and we sorted the top 10% and bottom 10% of GFP-expressing cells by FACS (FIG. 16B). We performed next-generation sequencing (NGS) to identify gRNAs enriched in either group. When we compared the low GFP expressing cells to unsorted cells, no hits emerged, indicating this population of cells lacked PAX7 expression altogether. When we compared high GFP expressing cells to unsorted cells, 10 unique genes (not including PAX7 gRNAs) emerged as significant (FIG. 16C). These gRNAs were individually cloned into lentiviral vectors and validated in the same cell line with the 2 week differentiation protocol (FIG. 16D). We also cloned the equivalent cDNA into lentiviral constructs and determined that protein delivery could also result in activation of PAX7, albeit to varying degrees (FIG. 16E).

Combinatorial CRa-TF Screen to Identify TFs Synergistic with PAX7. Although mesodermal differentiation with small molecules has been shown to generate myogenic cells, it also leads to differentiation of heterogenous cell types including neurons. Mesodermal differentiation with CHIR99021 is also used for differentiation of pluripotent cells into cardiac and kidney lineages as well. It has previously been demonstrated that PAX7 cDNA expression during the differentiation time-course can influence cells to adopt a myogenic cell fate over alternative lineages.

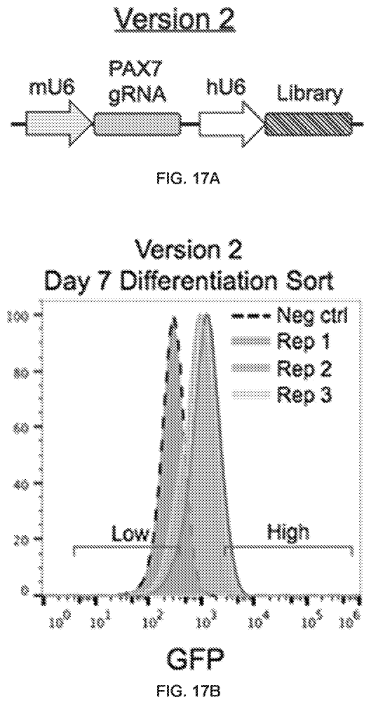

We performed a second screen with the addition of a mU6-PAX7 promoter-targeting gRNA cassette in the lentiviral CRa-TF library (FIG. 17A). This screen also has the potential to identify TFs that work synergistically with PAX7 to enhance myogenic progenitor cell specification. We performed the screen as described earlier, except we reduced the differentiation to 1 week rather than 2 weeks since we anticipated rapid upregulation of PAX7. After 1 week of differentiation we saw a clear shift in the GFP population and sorted the top 10% and bottom 10% of GFP expressing cells (FIG. 17B). This second screen uncovered 13 IFs that when co-expressed with PAX7, creates an additive effect on PAX7 expression. In total, both screens yielded a list of 21 IFs that upregulate PAX7 in the context of mesoderm differentiation (FIG. 17C).

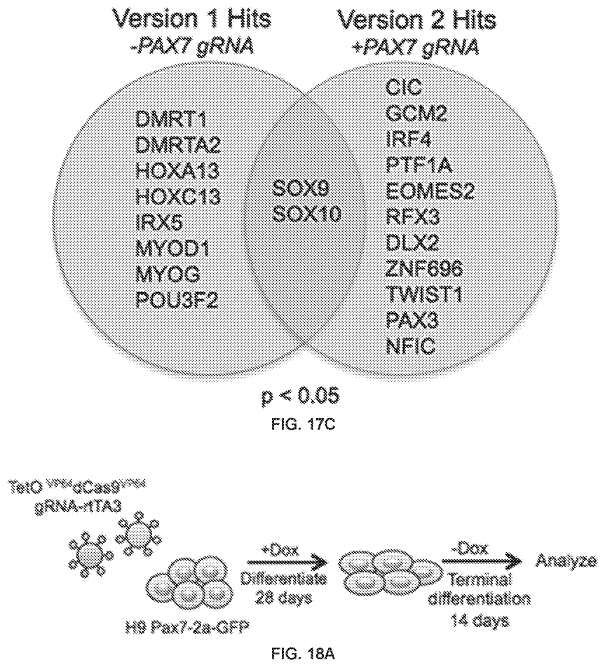

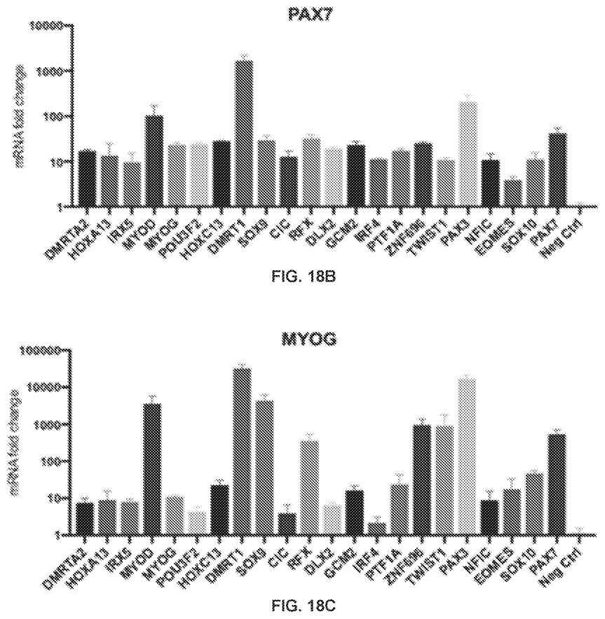

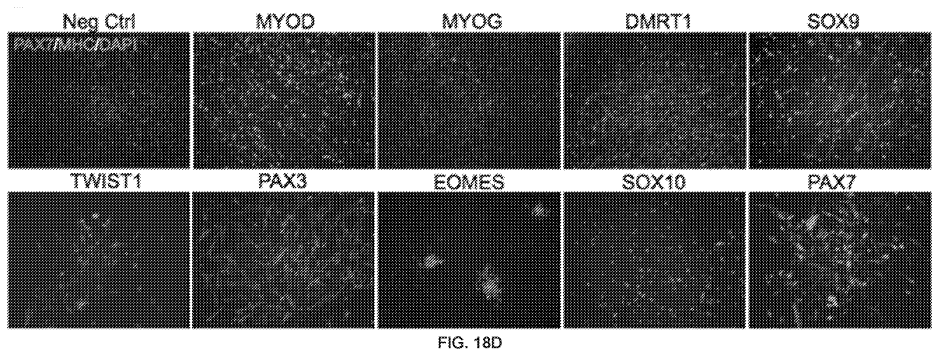

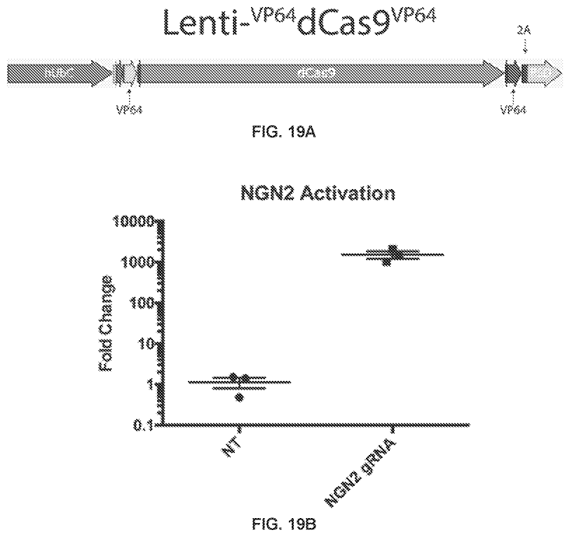

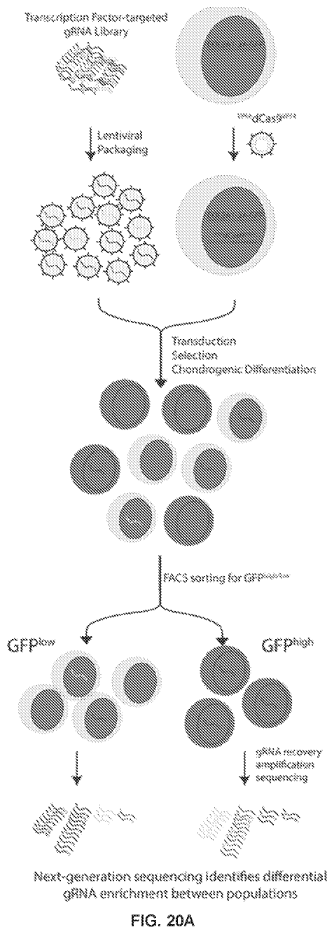

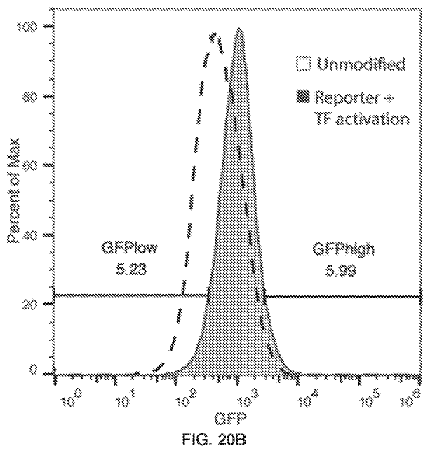

Validation of Hit TFs that Promote Myogenic Differentiation. Next, we wanted to determine if the TFs could not only upregulate PAX7 expression, but also yield myogenic cells. We cloned each of the 21 TF gRNA hits into a lentiviral vector expressing rtTA3 and used a tetracycline-inducible promoter to drive expression of ^{VP64}dCas9^{VP54}. We transduced both constructs into the H9 PAX7-2a-GFP cell line and differentiated the cells in the presence of doxycycline (dox) for 28 days with a passaging step at day 14. We withdrew dox after 28 days to allow for downregulation of PAX7, which allows downstream myogenic genes to become upregulated to induce terminal differentiation of myogenic progenitors into myocytes (FIG. 18A). qRT-PCR analysis showed slightly upregulated PAX7 expression in many of the conditions after 2 weeks of terminal differentiation compared to a scramble gRNA control. Surprisingly, three TFs, MYOD, DMRT1, and PAX3, demonstrated higher expression of PAX7 when compared to the PAX7 gRNA-expressing control (FIG. 18B). We also examined expression of the downstream myogenic marker, MYOG, and found it was highly expressed in 8 of the 21 novel TF gRNA hits (FIG. 18C). Lastly, we performed immunofluorescence staining of fixed differentiated cells for presence of myosin-heavy chain (MHC) positive myofibers (FIG. 18D). We also stained for PAX7 to determine if any of the novel hits could generate a cell type that could sustain a PAX7+ satellite cell phenotype. Many of the putative hits expressing MYOG also displayed presence of MHC+ myofibers, DMRT1 displayed the highest number of PAX7+ nuclei and generated myofibers most robustly.

Discussion. In this study, we use an unbiased systematic approach to screen all human TFs for myogenic progenitor cell fate specification. Using PAX7 expression as a proxy for satellite cell specification, we generated a PAX7-2a-GFP human embryonic stem cell line to uncover novel upstream regulators of PAX7 during the course of myogenic differentiation. Using individual and combinatorial CRISPRa screens, we generated a list of 21 putative TFs that demonstrated activation of PAX7. A subset of these TFs also demonstrated the ability to differentiate ESCs into myofibers. Hits such as TWISTI and PAX3 were unsurprising due to their previously characterized importance for paraxial mesoderm development. PAX3 in particular is the paralogue of PAX7 they have overlapping functions as upstream regulators of myogenesis. MYOD and MYOG were interesting hits because they are understood to lie downstream of PAX7 expression during myogenesis. A likely explanation is that overexpression of these myogenic factors pushes embryonic stem cells toward the myogenic program to generate primary myofibers of the myotome, which may then form a positive feedback loop to generate more PAX7-derived embryonic myoblasts. In the two versions of the CRISPRa screens conducted in this study, SOX9 and SOX10 were the only TFs to emerge as hits in both. SOX9 and SOX10 are both important TFs during development and SOX factors in general are involved in cell fate determination. SOX9′s implications span from chondrogenesis to central nervous system development and it has also been shown to enhance differentiation of ESCs into progenitors of all 3 germ layers. Like SOX9 and PAX7. SOX10 also plays an important role in neural crest development. Unlike PAX7, SOX10 is not expressed in mesoderm; however, SOX10-deficient embryos exhibit a significant reduction in PAX7+ muscle progenitor cells and a reduced myotome formation. The combination of prior studies linking SOX9 and SOX10 to differentiation and proper myogenesis and the emergence of these TFs in our CRa-TF screen solidifies their importance in myogenic progenitor cell specification.

Of all the hits analyzed one TF in particular, DMRT1, showed the exciting ability to generate a multitude of PAX7+ cells among abundant myofibers in vitro. DMRT1 is a particularly unexpected hit because it is mainly recognized as a sex determination gene. This gene is predominantly expressed in Sertoli cells and is necessary for testicular maturation. Interestingly, PAX7 was recently identified as a marker for a rare subpopulation of spermatogonia in mice that have stem cell-like properties. Although there is no defined link between DMRT1 and PAX7 in the context of either spermatogenesis or myogenesis, our results would suggest that DMRT1 has the ability to act upstream of PAX7 and activate its expression to give cells a stem-cell phenotype. In the context of the mesodermal differentiation used in our screen, this gives rise to myogenic progenitor cells and myofiber generation. While this process may not be a naturally occurring phenomenon, DMRT1 overexpression may be harnessed for generating robust myogenic progenitors for cell therapies.

In conclusion, we performed a powerful CRISPRa screen of all human IFs, which revealed hits that were a combination of expected, intriguing, and surprising. These results shed light on our understanding of satellite cell development and the upstream regulators of PAX7 and can be useful for engineering myogenic progenitor cells. The approach developed in this study has broad utility for discovering novel TFs to enhance engineering of other cell lineages.

### Example 10

### Identification of Transcription Factors that Regulate Chondrogenesis

A high-throughput CRISPR activation screen similar to that detailed in Example 9 was used to identify novel drivers of chondrocyte-specific gene expression. A gene specifically expressed in collagen was used as the chondrocyte-specific marker. Chondrocyte-specific transcription factors were identified.

Generation of TF-targeted CRISPR Activation Library. gRNAs targeting annotated TFs as described in the previous Examples were extracted from the library, resulting in a library comprised of 8,435 gRNAs (roughly 5 gRNAs per TF). The library was amplified and cloned into a modified lenti-CRISPR construct containing an mCherry-2A-Pure expression cassette using Gibson Assembly.

Lentiviral Production and Titration. Lentiviral packaging of gRNA library and VP64-dCas9-VP64 expression vector was performed by transfecting pooled gRNA library plasmids or VP64-dCas9-VP64 plasmid (20 μg), pMD2.G (Addgene, 12259, 6 μg), and psPAX2 (Addgene, 12260, 15 μg) into 3E6 HEK 293Ts using calcium phosphate precipitation. After 16 hours, media was replaced. Viral supernatant was collected 24 and 48 hours later and concentrated using Lenti-X concentration system (Clonetech) according to the manufacturer's instructions.

Titration of lentivirus containing gRNA library was performed by transduction of COL2A1-2A-GFP; VP64-dCas9-VP64 hiPSCs in a 24-well plate at 60K cells/cm² eight hours after plating. 10-fold serial dilutions of concentrated lentivirus, ranging from 5E-5 to 5 μL were added, were added to the media. Media was changed 16 hours after transduction and mCherry fluorescence was measured using BD Accuri C6 cytometer to determine transduction efficiency at D3.

Generation Validation of CRISPR activator hiPSC line. COL2A1-2A-GFP reporter hiPSCs were transduced with lentivirus carrying an expression cassette of dCas9 fused to VP64 transactivation domains at N- and C-termini as described above. Cells were selected with 100 μg/mL blasticidin for 5 days. The resulting polyclonal line was validated by transduction of NGN2-targeting gRNA. After 3 days, cells were lysed and NGN2 expression was assessed by qRT-PCR.

Gene expression. Cells in monolayer and pellets were rinsed with DPBS. Monolayer cells were lysed in 350 μl of Buffer RL (Norgen Biotek, Thorold Canada). The RNA was isolated using the Total RNA Purification Kit according to the manufacture's recommendations (Norgen Biotek). Reverse transcription was performed using SuperScript™ VILO™ Master Mix (Thermo Fisher) per the manufacturer's instructions. Quantitative RT-PCR was performed on the QuantStudio 3 (Thermo Fisher) and CFX96 Real Time System (Biorad, Hercules Calif.) using Fast SYBR™ Green Master Mix (Thermo Fisher) according to the manufacturer's protocol. Fold changes were calculated using the ΔΔC_{T} method relative to hiPSCs as the reference time point and TATA-box-binding protein (TBP) as the reference gene. Gene expression of NGN2 was assessed using the primer pair:

F:
(SEQ ID NO: 151)
5′-CAGGCCAAAGTCACAGCAAC-3′
R:
(SEQ ID NO: 152)
5′-CGATCCGAGCAGCACTAACA-3′

Lentiviral gRNA screening of TF-targeted library. To maintain >500-fold library coverage, 5 15-cm matrigel coated dishes containing 4.5×10⁶ million cells each were transduced with lentiviral gRNA library in 25 mL of complete mTeSR at an MOI of 0.2 to ensure that most cells contained 0 or 1 gRNA. Transduced cells were selected with 0.5 pg/mL Puromycin for 3 days and passage at density of 10K/cm² in 4 15-cm matrigel coated dishes. At this time point a sample of 5×10⁶ cells were sampled to serve an input control for each replicate. 24 hours after seeding cells were selected with puromycin for another 2 days to ensure complete selection. Cells were differentiated to chondroprogenitors as described in 2.4.3 for 21 days. At this timepoint, the top/bottom 5^{th} percentiles were collected in addition to an unsorted population. After sorting, input, unsorted, GFP^{high}, and GFP^{low} populations were harvested for genomic DNA purification (Qiagen).

gRNA library sequencing. gRNA libraries were amplified from each population by amplifying from 12 μg of gDNA split into twelve 100 μL FOR reactions using Q5 Hot-Start Polymerase (NEB, M0493L). We used the following PCR conditions: 60 degree annealing temperature, 20″ extension time, for 25 cycles. The following primers were used:

F:
(SEQ ID NO: 153)
5′ AATGATACGGCGACCACCGAGATCTACACAATTTCTTGGGTAGTTT
GCAGTT-3′
R:
(SEQ ID NO: 154)
5′-CAAGCAGAAGACGGCATACGAGAT(NNNNNN)GACTCGGTGCCACT
TTTTCAA-3′
where NNNNNN denotes 6-bp barcode sequence.

PCR-amplified libraries were purified using Agencourt AMPure XP beads (Beckman Coulter) using double selection to remove large fragments and primer dimers by first adding a bead volume of 0.65× PCR volume and then 1× original FOR volume. After resuspension in water, library concentrations in each sample was determined using the Qubit dsDNA High Sensitivity kit (ThermoFisher). Samples were pooled and 21-bp paired end sequencing was performed on Illumina Miseq using the following read and index primers:

Read 1:
(SEQ ID NO: 155)
5′-GATTTCTTGGCTTTATATATCTTGTGGAAAGGACGAAACACCG-3′
Read 2:
(SEQ ID NO: 156)
5′-GTTGATAACGGACTAGCCTTATTTTAACTTGCTATTTCTAGCTCTA
AAAC-3′
Index:
(SEQ ID NO: 157)
5′-GCTAGTCCGTTATCAACTTGAAAAAGTGGCACCGAGTC-3′

Analysis of differential gRNA enrichment. FASTQ files generated by MiSeq sequencing were aligned to custom indexes using Bowtie 2 with the options -p 32—end-to-end—very-sensitive −3 2-I 0-X 200. We then created a counts table for the number of reads of each gRNA in each sequenced population. Significant enrichment of each gRNA was assessed using the DESeq2 package in R. We compared unsorted to GFP^{high}, unsorted to GFP^{low}, and GFP^{high} to GFP^{low}; here we only show data for the GFP^{high} to GFP^{low} comparison.

Validation of candidate TFs. Reporter hiPSCs were transduced with lentivirus containing SOX9 cDNA as described in 4.4.3 alongside non-transduced controls. After two days of recovery, cells were differentiated according to the chondrogenic protocol described in 2.4.2 but harvested at the sclerotome stage (D6). At this time point, chondrogenic differentiation was evaluated using flow cytometry with Accuri C6 cytometer.

Identification of Candidate Regulators of hiPSC Chondrogenesis. To evaluate the effect of activated TFs on chondrogenic differentiation, we generated, in the COL2A1-2A-GFP background, a line stably expressing dCas9 fused to VP64 transactivation domains at both N- and C-terminals (VP64-dCas9-VP64) (FIG. 19A). Transduced cells were selected to generate a polyclonal activator line. This polyclonal line robustly activated endogenous Neurogenin 2 (NGN2) after transduction of gRNA targeting its promoter (FIG. 19B).

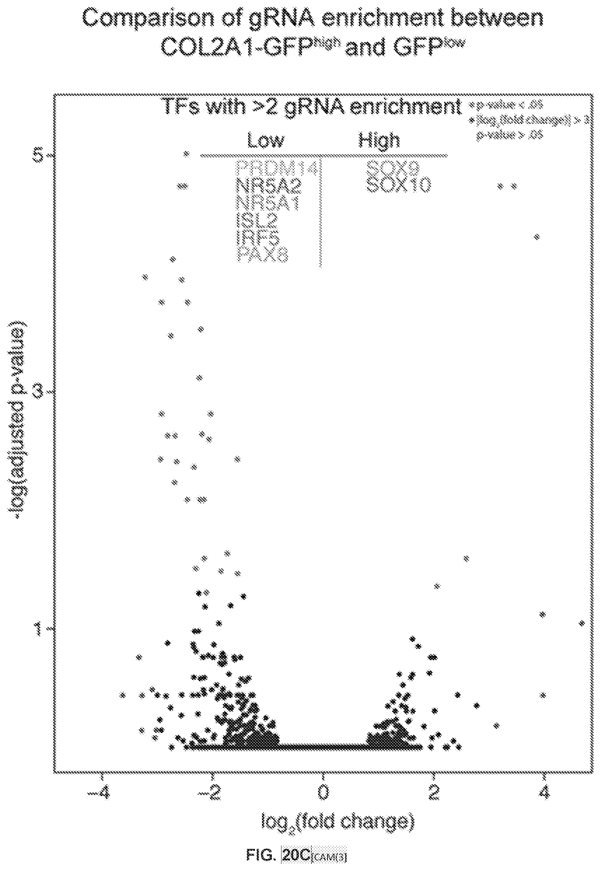

To generate a TF-targeted CRISPR activation library, we extracted TF-targeting gRNAs from a previously described, publicly available, genome-scale activation library as similarly detailed in Example 9. The gRNA library was cloned into a Lenti-CRISPR construct harboring an mCherry-2a-Pure^{R} expression cassette to allow selection of transduced lines (FIG. 20A). Transduction of Lenti-CRISPR library at low multiplicity of infection (MOD into our activator reporter line ensured one gRNA per cell, and adequate coverage (>500×) of the library was maintained. Transduced cells were then differentiated (FIG. 20A). Transduction of the gRNA library seemed to eliminate the bimodal distribution of GFP at day 21; nevertheless, GFP^{high/low} populations were sorted (FIG. 20B). We observed significant (adjusted p-value<0.05) differential enrichment of 36 gRNAs (FIG. 20C).

Notably two gRNAs targeting SOX9 were significantly enriched in the GFP^{high} population. We also observed strong enrichment for two gRNAs targeting SOX10, another transcription factor known to be involved in limb bud chondrogenesis. The roles of SOX15 and TBR1, remain to be validated and defined. Interestingly, several more gRNAs were enriched in the GFP^{low} population. As expected, gRNAs targeting TFs strongly expressed in the pluripotent state, such as PRDM14 and NR5A2, were enriched in this population. However, other commonly cited pluripotency TFs such as NANOG and OCT4 were not enriched in this population. Surprisingly, gRNAs targeting TFs that are induced during chondrogenesis, such as P17X1, HES1, 1D4, SP9, and SIX6, were also enriched in the GFP^{low} population. gRNAs enriched over 3-fold in either population, but not meeting significance criteria, are colored in blue (FIG. 20C).

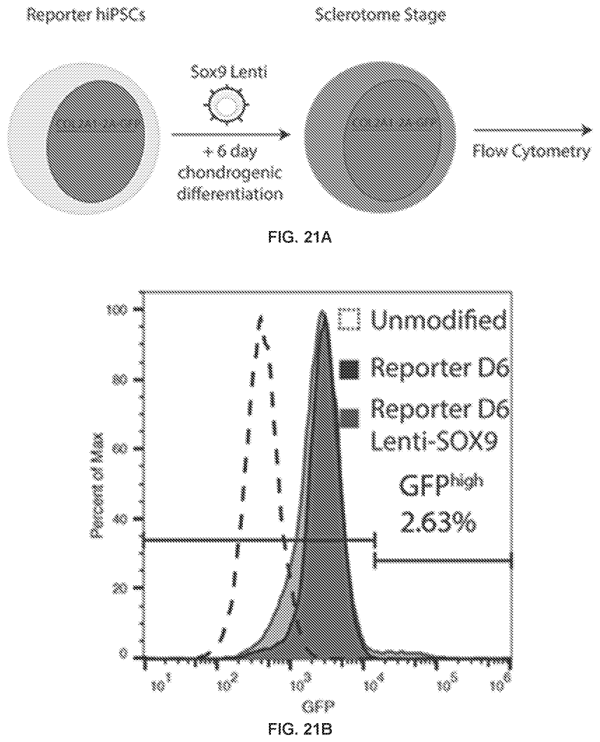

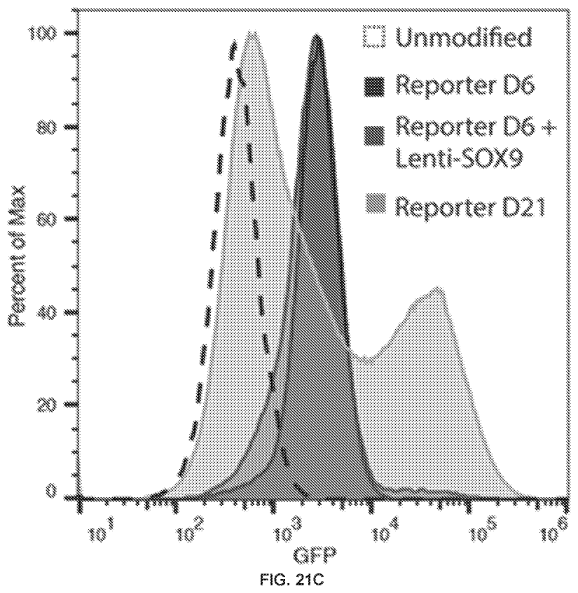

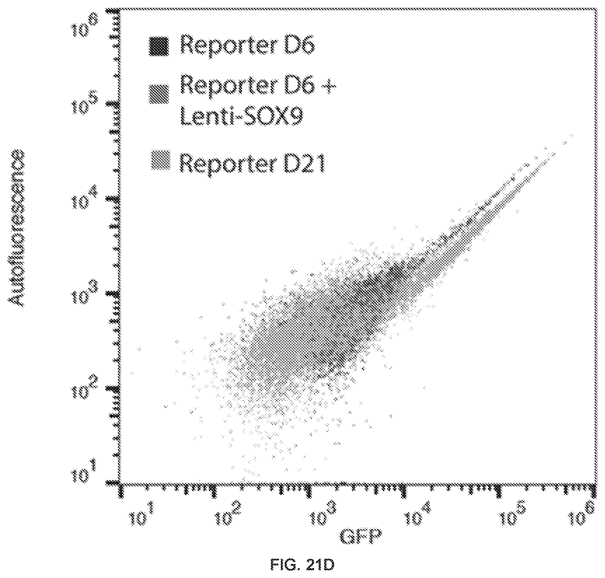

Preliminary Validation of Screening Results by SOX9 Overexpression. While SOX9 is a known chondrogenic transcription factor that binds directly to promoter and enhancer elements of genes encoding cartilage matrix proteins, it was unclear what effect SOX9 activation would have in the context of our staged differentiation. Gene expression data from time course experiments suggested that SOX9 activation occurs at D12 of this differentiation protocol. To determine the effect of SOX9 overexpression on chondrogenesis in the context of our differentiation scheme, we transduced lentivirus encoding SOX9 cDNA to reporter hiPSCs and assessed reporter fluorescence after 6 days of differentiation (FIG. 21A). At this stage, cells have not yet been exposed to chondrogenic growth factor BMP-4, and the establishment of protocol that bypasses the need for the lengthy (6-15 day) pre-chondrogenic differentiation in monolayer would be valuable. Indeed, much of the variability that we observed in our chondrogenic differentiation protocol occurs at this stage of differentiation.

After 6 days of differentiation with SOX9 overexpression and prior to any BMP-4 treatment, we observed a GFP^{high} population of roughly 2-3% of the total population (FIG. 21B). SOX9 transduction also seemed to broaden the distribution of reporter fluorescence to the left. Fluorescence intensity of this population generated by SOX9 overexpression was comparable to that of reporter cells at day 21 of differentiation, though the proportion of these cells was considerably lower (FIG. 21C).

Discussion. Here, we show a high-throughput screen of all TFs for their ability to regulate chondrogenesis. SOX9, which we expected to be enriched in the GFP^{high}, population served as an internal control. Other factors known to be involved in chondrogenesis such as SOX10 were also enriched in the GFP^{high} population. SOX10 has been shown to be involved in limb bud chondrogenesis and coordinates the chondrogenic program along with SOX9 and SOX8, and may be involved promoting hypertrophic differentiation of chondrocytes. A potential role of TBR1 and SOX15 for chondrogenesis may be less clear; SOX15 has been implicated in muscle regeneration, and TBR1 is known to be expressed glutamatergic neurons.

Our screen generated far more hits that were enriched in the GFP^{low} population, Strong activation of most TFs might impede chondrogenic, specification at various stages of differentiation. The most significantly enriched gRNAs in this population target PRDM14, a regulator of naïve pluripotency. gRNAs targeting NR5A2, also highly expressed in pluripotency, are also enriched in this population. Notably gRNAs targeting TFs that are involved in and activated during chondrogenesis, such as PITX1, are also enriched in the GFP^{low}.

In our validation experiment to test SOX9 overexpression in the context of differentiation, we observed, after 6 days of differentiation, the emergence of a GFP^{high} population prior to the addition of BMP-4, suggesting that exogenous delivery of TFs may bypass the pre-chondrogenic phase of differentiation. It appears that hiPSC-derived sclerotome was appropriately poised to activate COL2A1 in response to SOX9. Close analysis of the histogram shown in FIG. 21B reveals that overexpression of SOX9, in addition to generating a GFP^{high}, seems to increase the height of the left tail of histogram, which suggests overexpression of SOX9 may also be inhibiting chondrogenic differentiation in a subset of cells.

In summary, we demonstrate the utility of a high-throughput hiPSC chondrogenesis platform using a COL2A1 knock-in reporter to screen pro-chondrogenic TFs. The screen successfully enriched gRNAs targeting the known chondrogenic TF SOX9 and produced several other interesting hits. The TFs discovered herein may improve techniques to generate hiPSC-derived cartilage or to specific various chondrocyte subtypes (such as articular versus growth plate).

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A polynucleotide encoding: (1) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLlG3, HIC1, SOX3, FOXJ1, SOX10, KLF6, ASCL1, and PLAGL2: or (2) a first neuronal-specific transcription factor selected from NGN3 and ASCL1, or a combination thereof; and a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLlG3, HIC1, SOX3, FOXJ1 SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3; (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7; (iv) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (v) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791; (vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1,1RF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX.

Clause 2. A system for increasing expression of a neuronal-specific gene, the system comprising: (a) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; or (b) a first gRNA targeting a first neuronal-specific transcription factor selected from NGN3 and ASCL1, or a combination thereof; and a second gRNA targeting a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3; (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAXS, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7; (iv) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (v) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791; (vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX; and a Cas protein or a fusion protein, wherein the fusion protein comprises two heterologous polypeptide domains, wherein the first polypeptide domain comprises a Cas protein, a zinc finger protein, or a TALE protein, and the second polypeptide domain has an activity selected from transcription activation activity, transcription repression activity, transcription release factor activity, histone modification activity, nuclease activity, nucleic acid association activity, methylase activity, and demethylase activity.

Clause 3. The polynucleotide of clause 1 or the system of clause 2, wherein the second neuronal-specific transcription factor is selected from LHX8, LHX6, E2F7, RUNX3, FOXH1, SOX2, HMX2, NKX2-2, HES3, and ZFP36L1.

Clause 4. The polynucleotide or system of clause 3, wherein the second neuronal-specific transcription factor is selected from LHX8, LHX6, E2F7, RUNX3, FOXH1, SOX2, HMX2, and NKX2-2.

Clause 5. The polynucleotide or system of clause 3, wherein the second neuronal-specific transcription factor is selected from HES3 and ZFP36L1.

Clause 6. The system of clause 2, wherein the second neuronal-specific transcription factor is selected from: (i) NEUROG3, SOX4, SOX9, KLF4, NR5A1, NEURODI, SOX17, SMAD1, ATOH1, INSM1, NEUROG1, SOX18, RFX4, KLF7, SF8, OVOL1, NEUROG2, ERF, PRDM1, OLIG3, HIC1, SOX3, FOXJ1, SOXIO, KLF6, ASCL1, and PLAGL2, (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3; (iii) RUNX3, PRDM1, KLF6, PAX2, RFX3, SOX10, GATA1, KLFS, KLF1, ERF, LHX6, PHOX2B, NANOG, NR5A2, ETV3, NEUROG3, SOX4, SOX9, PAX8, !RFS, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1 OLIG3, HMX3, ZNF521, ONECUT3, OVOL3, ZNF362, AFF1. HMX2, ZNF786, GATA5, TBX3, ZNF385A, ATOH1, PROP1. SOX11, JUN, FOXE3, FERD3L, and E2F7, and wherein the second polypeptide domain has transcription activation activity.

Clause 7. The system of clause 6 erein the fusion protein comprises ^{VP64}dCas9^{VP64} or dCas9-p300.

Clause 8. The system of clause 2, wherein the second neuronal-specific transcription factor is selected from: (i) ZIC2, SP11, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VDR, HES1, ID2, TCF21, SNAI1, RREB1, GCM2, 1RF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMPIL. TOX3, FEZF2, HES3, ZNF791: (iii) ETV1, Z1C2, GSC2, CIC, GRHL2. REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETVS, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1 L, ZNF821, KMT2A, HESS, and BSX, and wherein the second polypeptide domain has transcription repression activity,

Clause 9. The system of clause 8, wherein the fusion protein comprises dCas9-KRAB.

Clause 10, The system of any one of clauses 2-9, wherein the first gRNA and the second gRNA each individually comprise a 12-22 base pair complementary polynucleotide sequence of the target DNA sequence followed by a protospacer-adjacent motif, and optionally wherein the gRNA binds and targets and/or comprises a polynucleotide comprising a sequence selected from SEQ ID NOs: 38-87, and optionally wherein the first and/or second gRNA comprises a crRNA, a tracrRNA, or a combination thereof.

Clause 11. An isolated polynucleotide encoding the system of any one of clauses 2-10.

Clause 12. A vector comprising the isolated polynucleotide of clause 11.

Clause 13. A cell comprising the isolated polynucleotide of clause 11 or the vector of clause 12.

Clause 14. A method of increasing maturation of a stem cell-derived neuron, the method comprising: (a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2, or (b) increasing in the stern cell the level of a first neuronal-specific transcription factor selected from NGN3 and ASCL1, or a combination thereof; and increasing in the stern cell the level of a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3, (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFl1, KLF17, OVOL1, OLIG3, HMX3, ZNF521, ONECUT3, OVOL3, ZNF362, AFF1, HMX2, ZNF786, GATAS, TBX3, ZNF385A, ATOH1, PROP1, SOX11, JUN, FOXE3, FERD3L, and E2F7.

Clause 15. A method of increasing maturation of a stem cell-derived neuron, the method comprising: increasing in the stem cell the level of a first neuronal-specific transcription factor selected from NGN3 and ASCL1, or a combination thereof; and decreasing in the stem cell the level of a second neuronal-specific transcription factor selected from: (i) ZIC2, SPI1, GRHL2, TFAP2C, KLFB, MYB, TCF21, KLF12, TWIST1, SNAIL RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VIER, HES1, ID2, TCF21, SNAI1, RREB1, GCM2, IRF3, FOXA1, GATAS, GRHL1, SOXS, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF71O, ZNF697, ZFP36L2, ELMSAN1, ZNF296, ZNF318, ZNF570, ZNF683, ZFP36L1, HES4, ZNF777, HESS, ZIM2, ZNF579, BMP2, CRAMP1L, TOX3, FEZF2, HES3, ZNF791; (iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOXS, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L, ZNF821, KMT2A, HES3, and BSX.

Clause 16. A method of increasing the conversion of a stem cell to a neuron, the method comprising: (a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SPS, OVOL1, NEUROG2, ERF, PRDM1, OLIG3, HIC1, SOX3, FOXJ1, SOX10, KLF6, ASCL1, and PLAGL2, or (b) increasing in the stem cell the level of a first neuronal-specific transcription factor selected from NGN3 and ASCL1, or a combination thereof; and increasing in the stem cell the level of a second neuronal-specific transcription factor selected from: (i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAXS, KLF3; (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SP1B, THRB, FOXH1, NEUROD1, SOX17, CDX2, ZEB2, RARG, INSM1 , FOSL1, NEUROG1, SOX1, WT1, PAX5, SOX18, POU5F1, RFX4, KLF7, NKX2-2, OVOL2, FOXJ1, PRDM14, VENTX. LHX8, GFI1, KLF17. OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

Clause 17. A method of increasing the conversion of a stem cell to a neuron, the method comprising: increasing in the stem cell the level of a first neuronal-specific transcription factor selected from NGN3 and ASCL1, or a combination thereof; and decreasing in the stern cell the level of a second neuronal-specific transcription factor selected from: (i) ZIC2, SPI1, GRHL2, TFAP2C, KLF8, MYB, TCF21, KLF12, TWIST1, SNAI1, RREB1, GCM2, GRHL1. ETS1, BARHL2, GRHL3, ELF3, PTF1A, GSX1, PBX2, NOTO, KLF3, ZNF311, ELMSAN1, ZNF296, PLEK. KMT2A, HES3; (ii) HES2, SREBF1, CIC, WHSC1. VDR, HES1, ID2, TCF21, SNAI1, RREB1. GCM2. IRF3, FOXA1, GATA5, GRHL1, SOXS, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791; (iii) ETV1, ZIC2, GSC2, CIC, GRHL2, REST, TFAP2C, SALL1, NFKB1, ELF2, HES1, MYB, KLF12, VSX2, NFE2, SNA11, TRERF1, RREB1,1RF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, P1TX2, PTF1A, GSX1, ZNF160, ETV5, MYBLI, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1. HESS, BMP2, CRAMP1L, ZNF821, KMT2A, HES3, and BSX.

Clause 18. A method of treating a subject in need thereof, the method comprising: (a) increasing in a stem cell in the subject the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, H1C1, SOX3, FOXJ1, SOX10, KLF6, ASCL1, and PLAGL2, or (b) increasing in a stern cell in the subject the level of a first neuronal-specific transcription factor selected from NGN3 and ASCL1, or a combination thereof; and increasing in a stem cell in the subject the level of a second neuronal-specific transcription factor selected from: (i) NEUROG3, SOX4, SOX9, KLF4, NR5A1, NEUROD1, SOX17, SMAD1, ATOH1, INSM1, NEUROGI SOX18, RFX4, KLF7, SP8, OVOL1, NEUROG2, ERF, PRDM1, OLIG3, HIC1, SOX3, FOXJ1, SOX10, KLF6, ASCL1, and PLAGL2; (ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3; (iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8,1RF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

Clause 19. A method of treating a subject in need thereof, the method comprising: increasing in a stem cell in the subject the level of a first neuronal-specific transcription factor selected from NGN3 and ASCL1, or a combination thereof; and decreasing in a stem cell in the subject the level of a second neuronal-specific transcription factor selected from: (i) ZIC2, SPII, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3; (ii) HES2, SREBF1, CIC, WHSC1, VCR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791; (iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L, ZNF821, KMT2A, HES3, and BSX.

Clause 20, The method of any one of clauses 14-19, wherein increasing the level of the first neuronal-specific transcription factor comprises at least one of: (a) administering to the stem cell a polynucleotide encoding the first neuronal-specific transcription factor; (b) administering to the stem cell a polypeptide comprising the first neuronal-specific transcription factor; and (c) administering to the stem cell a fusion protein, wherein the fusion protein comprises two heterologous polypeptide domains, wherein the first polypeptide domain comprises a Cas protein, a zinc finger protein targeting the first neuronal-specific transcription factor, or a TALE protein targeting the first neuronal-specific transcription factor, and the second polypeptide domain has transcription activation activity, and wherein a gRNA targeting the first neuronal-specific transcription factor is additionally administered to the stem cell when the first polypeptide domain comprises a Cas protein.

Clause 21. The method of any one of clauses 14, 16, and 18, wherein increasing the level of the second neuronal-specific transcription factor comprises at least one of: (a) administering to the stern cell a polynucleotide encoding the second neuronal-specific transcription factor; (b) administering to the stem cell a polypeptide comprising the second neuronal-specific transcription factor; and (c) administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription activation activity, and wherein a gRNA targeting the second neuronal-specific transcription factor is additionally administered to the stem cell when the first polypeptide domain comprises a Cas protein.

Clause 22. The method of any one of clauses 15, 17, and 19, wherein decreasing the level of the second neuronal-specific transcription factor comprises administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription repression activity, and wherein a gRNA targeting the second neuronal-specific transcription factor is additionally administered to the stem cell when the first polypeptide domain comprises a Cas protein.

Clause 23. The method of any one of clauses 14-22, wherein the stem cell is directly converted to a neuron without a pluripotent stage.

Clause 24. The cell of clause 13 or the method of any one of clauses 14-23, wherein the stem cell is a pluripotent stern cell, an induced pluripotent stem cell, or an embryonic stern cell.

Clause 25. A system for selecting a polynucleotide for activity as a cell type-specific transcription factor, the system comprising: a polynucleotide encoding a reporter protein and a cell type marker; a fusion protein, wherein the fusion protein comprises two heterologous polypeptide domains, wherein the first polypeptide domain comprises a Cas protein, and the second polypeptide domain has transcription activation activity; and a library of guide RNAs (gRNAs), each gRNA targeting a different putative cell type-specific transcription factor.

Clause 26. The system of clause 25, wherein the cell-type specific transcription factor is a neuronal-specific transcription factor, wherein the cell type marker is a neuronal marker, and wherein the neuronal marker comprises TUBB3.

Clause 27. The system of clause 25, wherein the cell-type specific transcription factor is a muscle-specific transcription factor, wherein the cell type marker is a myogenic marker, and wherein the myogenic marker comprises PAX7.

Clause 28. The system of clause 25, wherein the cell-type specific transcription factor is a chondrocyte-specific transcription factor, wherein the cell type marker is a collagen marker, and wherein the collagen marker comprises COL2A1,

Clause 29. The system of any one of clauses 25-28, wherein the reporter protein comprises mCherry.

Clause 30. An isolated polynucleotide sequence encoding the system of any one of clauses 25-29.

Clause 31. A vector comprising the isolated polynucleotide sequence of clause 30.

Clause 32. A cell comprising the system of any one of clauses 25-29, the isolated polynucleotide sequence of clause 30, or the vector of clause 31, or a combination thereof.

Clause 33. A method of screening for a cell type-specific transcription factor, the method comprising; transducing a population of cells with the system of any one of clauses 25-29 at a multiplicity of infection (MOD) of about 0.2, such that a majority of the cells each independently includes one gRNA and targets one putative transcription factor; determining a level of expression of the reporter protein in each cell; determining a level of the gRNA in each cell having a high expression of the reporter protein, wherein high expression of the reporter protein is defined as being in the top 5% among the population of cells; and selecting the putative transcription factor as a cell-type-specific transcription factor when the putative transcription factor corresponds to at least two gRNAs enriched in the cell having a high expression of the reporter protein.

Clause 34. A method of screening for a pair of cell-type-specific transcription factors, the method comprising: transducing a population of cells with the system of any one of clauses 25-29 at a multiplicity of infection (MOI) of about 0.2, such that a majority of the cells each independently includes two gRNAs and targets two putative transcription factors; determining a level of expression of the reporter protein in each cell; determining a level of the two gRNAs in each cell having a high expression of the reporter protein, wherein high expression of the reporter protein is defined as being in the top 5% among the population of cells; and selecting the two putative transcription factors as a pair of cell type-specific transcription factors when the putative transcription factors correspond to at least two gRNAs enriched in the cell having a high expression of the reporter protein.

Clause 35. The method of clause 33 or 34, wherein the level of expression of the reporter protein in each cell is determined after about four days from transduction.

Clause 36. The method of any one of clauses 33-35, wherein the level of expression of the reporter protein in each cell is determined by flow cytometry.

Clause 37, The method of any one of clauses 33-36, wherein the level of the gRNA in each cell having a high expression of the reporter protein is determined by deep sequencing.

Clause 38. The method of any one of clauses 33-37, wherein the gRNA increases the expression of the reporter protein in the cell by about 2-50% relative to a non-targeting gRNA.

Clause 39. A polynucleotide encoding a muscle-specific transcription factor selected from TWIST1, PAX3, MYOD, MYOG, SOX9, SOX10, and DMRT1.

Clause 40. A system for increasing expression of a muscle-specific gene, the system comprising: (a) a muscle-specific transcription factor selected from TWIST1, PAX3, MYOD, MYOG, SOX9, SOX10, and DMRT1; or (b) a fusion protein, wherein the fusion protein comprises two heterologous polypeptide domains, wherein the first polypeptide domain comprises a Cas protein, a zinc finger protein targeting a muscle-specific transcription factor selected from TWIST1, PAX3, MYOD, MYOG, SOX9, SOX10, and DMRT1, or a TALE protein targeting a muscle-specific transcription factor selected from TWIST1, PAX3, MYOD, MYOG, SOX9, SOX10, and DMRT1, wherein the second polypeptide domain has an activity selected from transcription activation activity, transcription release factor activity, histone modification activity, nucleic acid association activity, methylase activity, and demethylase activity, and wherein the system further includes a gRNA targeting a muscle-specific transcription factor selected from TWIST1, PAX3, MYOD, MYOG, SOX9, SOX10, and DMRT1 when the first polypeptide domain comprises a Cas protein.

Clause 41. The system of clause 40, wherein the fusion protein comprises ^{VP64}dCas9^{VP64} or dCas9-p300,

Clause 42. An isolated polynucleotide encoding the system of any one of clauses 40-41.

Clause 43. A vector comprising the isolated polynucleotide of clause 42.

Clause 44. A cell comprising the isolated polynucleotide of clause 42 or the vector of clause 43.

Clause 45. A method of increasing differentiation of a stem cell into a myoblast, the method comprising: increasing in the stem cell the level of a muscle-specific transcription factor selected from TWIST1, PAX3, MYOD, MYOG, SOX9, SOX10, and DMRT1.

Clause 46. A method of treating a subject in need thereof, the method comprising: increasing in a stem cell from the subject the level of a muscle-specific transcription factor selected from TWIST1, PAX3, MYOD, MYOG, SOX9, SOX10, and DMRT1.

Clause 47. The method of clause 45 or 46, wherein increasing the level of the muscle-specific transcription factor comprises at least one of: (a) administering to the stern cell a polynucleotide encoding the muscle-specific transcription factor; (b) administering to the stem cell a polypeptide comprising the muscle-specific transcription factor; and (c) administering to the stern cell a fusion protein, wherein the fusion protein comprises two heterologous polypeptide domains, wherein the first polypeptide domain comprises a Cas protein, a zinc finger protein targeting the muscle-specific transcription factor, or a TALE protein targeting the muscle-specific transcription factor, wherein the second polypeptide domain has transcription activation activity, and wherein a gRNA targeting the muscle-specific transcription factor is additionally administered when the first polypeptide domain comprises a Cas protein,

SEQUENCES

SEQ ID NO: 1
NGG (N can be any nucleotide residue, e.g., any of A, G, C, or T)
SEQ ID NO: 2
NGA (N can be any nucleotide residue, e.g., any of A, G, C, or T)
SEQID NO: 3
NGAN (N can be any nucleotide residue, e.g., any of A, G, C, or T)
SEQ ID NO: 4
NGNG (N can be any nucleotide residue, e.g., any of A, G, C, or T)
SEQ ID NO: 5
NGGNG (N can be any nucleotide residue, e.g., any of A, G, C, or T)
SEQ ID NO: 6
NNAGAAW (W = A or T; N can be any nucleotide residue, e.g., any of A, G, C, or T)
SEQ ID NO: 7
NAAR (R = A or G; N can be any nucleotide residue, e.g., any of A, G, C, or T )
SEQ ID NO: 8
NNGRR (R = A or G; N can be any nucleotide residue, e.g., any of A, G, C, or T)
SEQ ID NO: 9
NNGRRN (R = A or G; N can be any nucleotide residue, e.g., any of A, G, C, or T)
SEQ ID NO: 10
NNGRRT (R = A or G: N can be any nucleotide residue, e.g., any of A, G, C, or T)
SEQ ID NO: 11
NNGRRV (R = A or G; N can be any nucleotide residue, e.g., any of A, G, C, or T)
SEQ ID NO: 12
NNNNGATT (N can be any nucleotide residue, e.g., any of A, G, C, or T)
SEQ ID NO: 13
NNNNGNNN (N can be any nucleotide residue, e.g., any of A, G, C, or T)
SEQ ID NO: 14
codon optimized polynucleotide encoding S. pyogenes Cas9
atggataaaa agtacaqcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atrgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aarrgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga
aacacggatcgacctctctc aactgggcgg cgactag
SEQ ID NO: 15
Amino acid sequence of codon optimized poiynucleotide encoding S. pyogenes Cas9
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKEHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIAISLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DELKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 16
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tcccccctgg aggacctgct gaacaatcca ttcaactacg aggtcgatca tattatcccc
accagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatLLgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 17
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccac ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccad gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 18
codon optimized nucleic acid sequences encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctccd tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 19
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaddaaagaddaag
SEQ ID NO: 20
codon optimized nucleic acid sequences encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgdactac cgcmaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 21
Amino acid sequence of codon optimized nucleic acid sequence encoding S. aureus Cas9
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWDKIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNYLNGLYDKDNDKL
KKLINKSPEKLLMYHHDPQTYQKLKLMIEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 22
Polynucleotide sequence of D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagad gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct daaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 23
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 24
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtqaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
aggacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 25
codon optimized nucleic acid sequences encoding S. aureus Cas9
aagcggaactacatcctgggcctggacatcggcatcaccagcgtgggctacggcateatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaageaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacaq
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaacagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 26
Amino acid sequence of codon optimized nucleic acid sequences encoding S. aureus Cas9
KRNYILGLDIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQ
ISRNSKALEEKYVAELQLERLKKDGEVRGSINRFKTSDYVKEAKQLLKVQKAYHQLDQSFIDTYIDLL
ETRPTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPIIKKIKYYGN
KLNAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYLTVKSKKHPQIIKKG
SEQ ID NO: 27
Vector (pD0242) encoding codon optimized nucleic add sequences encoding S. aureus
Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggegtgtaeggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTAGGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGGATCAAAGTGATCAACGCCATCATCAAGAAGTACGGCCTGCCCAATGATATCATTATC
GAGCTGGCTAGGGAGAAGAACAGCAAGGACGGACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTAGCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaaqatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatctcttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 28
MCherry polypeptide
MVSKGEEDNMAIIKEFMRFKVHMEGSVNGHEFEIEGEGEGRPYEGTQTAKLKVTKGGPLPFAWDILSP
QFMYGSGAYVKHPADIPDYLKLSFPEGFKWERVMNFEDGGVVTVTQDSSLQDGEFIYKVKLRGTNFPS
DGPVMQKKTMGWEASSERMYPEDGALKGEIKQRLKLKDGGHYDAEVKTTYKAKKPVQLPGAYNVNIKL
DITSHNEDYTIVEQYERAEGRHSTGGMDELYKPKKKRKVGGPKKKRKV
SEQ ID NO: 29
mCherry polynucleotide
atggtgagcaagggcgaggaggataacatggccatcatcaaggagttcatgcgcttcaaggtgcacat
ggagggctccgtgaacggccacgagttcgagatcgagggcgagggcgagggccgcccctacgagggca
cccagaccgccaagctgaaggtgaccaagggcggccccctgcccttcgcctgggacatcctgtcccct
cagttcatgtacggctccaaggcctacgtgaagcaccccgccgacatccccgactacttgaagctgtc
cttccccgagggcttcaagtgggagcgcgtgatgaacttcgaggacggcggcgtggtgaccgtgaccc
aggactcctccctgcaggacggcgagttcatctacaaggtgaagctgcgcggcaccaacttcccctcc
gacggccccgtaatgcagaagaagaccatgggctgggaggcctcctccgagcggatgtaccccgagga
cggcgccctgaagggcgagatcaagcagaggctgaagctgaaggacggcggccactacgacgctgagg
tcaagaccacctacaaggccaagaagcccgtgcagctgcccggcgcctacaacgtcaacatcaagttg
gacatcacctcccacaacgaggactacaccatcgtggaacagtacgaacgcgccgagggccgccactc
caccggcggcatggacgagctgtacaagcccaagaagaagaggaaggtgggtggccctaagaaaaaga
gaaaggtgtga
SEQ ID NO: 30
Fwd: 5′-AATGATACGGCGACCACCGAGATCTACACAATTTCTTGGGTAGTTTGCAGTT
SEQ ID NO: 31
Rev: 5′-CAAGCAGAAGACGGCATACGAGAT-(6-bp index sequence)-
GACTCGGTGCCACTTTTTCAA
SEQ ID NO: 32
Read1: 5′-GATTTCTTGGCTTTATATATCTTGTGGAAAGGACGAAACACCG
SEQ ID NO: 33
Index: 5′-GCTAGTCCGTTATCAACTTGAAAAAGTGGCACCGAGTC
SEQ ID NO: 34
Read2:
5′-GTTGATAACGGACTAGCCTTATTTAAACTTGCTATGCTGTTTCCAGCATAGCTCTTAAAC
SEQ ID NO: 35
tttn (N can be any nucleotide residue, e.g., any of A, G, C, or T)
SEQ ID NO: 36
VP64-dCas9-VP64 protein
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYKEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
SEQ ID NO: 37
VP64-dCas9-VP64 DNA
cgggctgacgcattggacgactttgatctggatatgctgggaagtgacgccctcgatgattttgacct
tgacatgcttggttcggatgcccttgatgactttgacctcgacatgctcggcagcgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggaqatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
atccacctgggcgaacCgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgcactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 159
Human p300 (with L553M mutation) protein
MAENVVEPGPPSAKRPKLSSPALSASASDGTDFGSLFDLEHDLPDELINSTELGLTNGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPEAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIKPAGFVCDGCLKKSARTRKENKFSAKR
LPSTRLGTFLENRVNDFLRRQNHPESGEVTYRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEBRLT
SAKELPYFEGDFWPNVLEESIKELEQEEEERKREENTSNESTBVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTKCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 160
Human p300 Core Effector protein (aa 1048-1664 of SEQ ID NO: 134)
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDOPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 158
Polynucleotide sequence of a gRNA scaffold
gttttagagctagaaatagcaagttaaaataaggctagtccgttatcaacttgaaaaagtgg
caccgagtcggtgcttttttt

1. A polynucleotide encoding:
(1) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; or
(2) a first neuronal-specific transcription factor selected from NGN3 and ASCL1, or a combination thereof; and a second neuronal-specific transcription factor selected from:
(i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2;
(ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3;
(iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHXB, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7;
(iv) ZIC2, SP11, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(v) HES2, SREBF1, GIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3. TBX22, ZNF331, EGR4, ZIC5, ZNF710. ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791;
(vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBLI, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L ZNF821, KMT2A, HES3, and BSX.

2. A system for increasing expression of a neuronal-specific gene, the system comprising:
(a) a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2; or
(b) a first gRNA targeting a first neuronal-specific transcription factor selected from NGN3 and ASCL1, or a combination thereof; and a second gRNA targeting a second neuronal-specific transcription factor selected from:
(i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HICI SOX3, FOXJ1, SOX10, KLF6, ASCL1, and PLAGL2;
(ii) PRDM1, LHX6, NEUROG3, PAX8, SOX3, KLF4, FLII, FOXH1, FEV, SOX17, FOS, INSM1, SOX2, WT1, SOX18, ZNF670, LHX8, OVOL1, E2F7, AFFI HMX2, MAZ, RARA, PROP1, FOSL1, PAX5, KLF3;
(iii) RUNX3, PRDM1, KLF6, PAX2, RFX3, SOX10, GATA1, KLFS, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIE, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, E2F7;
(iv) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(v) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HEST, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HESS, ZIM2, ZNF579, BMP2, CRAMP1L, TOX3, FEZF2, HES3, ZNF791;
(vi) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HESS, BMP2, CRAMP1L, ZNF821, KMT2A, HES3, and BSX; and
a Cas protein or a fusion protein,
wherein the fusion protein comprises two heterologous polypeptide domains, wherein the first polypeptide domain comprises a Cas protein, a zinc finger protein, or a TALE protein, and the second polypeptide domain has an activity selected from transcription activation activity, transcription repression activity, transcription release factor activity, histone modification activity, nuclease activity, nucleic acid association activity, methylase activity, and demethylase activity.

3. The polynucleotide of claim 1 or the system of claim 2, wherein the second neuronal-specific transcription factor is selected from LHX8, LHX6, E2F7, RUNX3, FOXH1, SOX2, HMX2, NKX2-2, HES3, and ZFP36L1.

4. The polynucleotide or system of claim 3, wherein the second neuronal-specific transcription factor is selected from LHX8, LHX6, E2F7, RUNX3, FOXH1, SOX2, HMX2, and NKX2-2.

5. The polynucleotide or system of claim 3, wherein the second neuronal-specific transcription factor is selected from HES3 and ZFP36L1.

6. The system of claim 2, wherein the second neuronal-specific transcription factor is selected from:
(i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2;
(ii) PRDM1, LHX6, NEUROG3, PAX8, SOX3, KLF4, FLI1 , FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3;
(iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, 0¹101_2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7, and
wherein the second polypeptide domain has transcription activation activity.

7. The system of claim 6, wherein the fusion protein comprises ^{VP64}dCas9^{VP64} or dCas9-p300.

8. The system of claim 2, wherein the second neuronal-specific transcription factor is selected from:
(i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(ii) HES2, SREBF1, CIC, WHSC1, UDR, HES1, ID2, TCF21, SNAI1, RREB1, GCM2, IRF3, FOXA1. GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791;
(iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMPIL, ZNF821, KMT2A, HES3, and BSX, and
wherein the second polypeptide domain has transcription repression activity.

9. The system of claim 8, wherein the fusion protein comprises dCas9-KRAB.

10. The system of any one of claims 2-9, wherein the first gRNA and the second gRNA each individually comprise a 12-22 base pair complementary polynucleotide sequence of the target DNA sequence followed by a protospacer-adjacent motif, and optionally wherein the gRNA binds and targets and/or comprises a polynucleotide comprising a sequence selected from SEQ ID NOs: 38-97, and optionally wherein the first and/or second gRNA comprises a crRNA, a tracrRNA, or a combination thereof.

11. An isolated polynucleotide encoding the system of any one of claims 2-10.

12. A vector comprising the isolated polynucleotide of claim 11.

13. A cell comprising the isolated polynucleotide of claim 11 or the vector of claim 12.

14. A method of increasing maturation of a stem cell-derived neuron, the method comprising:
(a) increasing in the stern cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2, or
(b) increasing in the stem cell the level of a first neuronal-specific transcription factor selected from NGN3 and ASCL1, or a combination thereof; and
increasing in the stem cell the level of a second neuronal-specific transcription factor selected from:
(i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2;
(ii) PRDM1, LHX6, NEUROG3, PAX8, SOX3, KLF4, FLI1 , FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3;
(iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, IRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

15. A method of increasing maturation of a stern cell-derived neuron, the method comprising:
increasing in the stem cell the level of a first neuronal-specific transcription factor selected from NGN3 and ASCL1, or a combination thereof; and
decreasing in the stem cell the level of a second neuronal-specific transcription factor selected from:
(i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(ii) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF33 EGR4, ZIC5, ZNF710, ZNF697, ZFP36L2, ELMSANI, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791;
(iii) ETV1. ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX.

16. A method of increasing the conversion of a stem cell to a neuron, the method comprising:
(a) increasing in the stem cell the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HIC1, SOX3, FOXJ1, SOX10, KLF6, ASCL1, and PLAGL2, or
(b) increasing in the stem cell the level of a first neuronal-specific transcription factor selected from NGN3 and ASCL1, or a combination thereof; and
increasing in the stem cell the level of a second neuronal-specific transcription factor selected from:
(i) NEUROG3, SOX4, SOX9, KLF4, NR5A1, NEUROD1, SOX17, SMAD1, ATOH1, INSM1, NEUROG1, SOX18, RFX4, KLF7, SPS, OVOL1, NEUROG2, ERF, PRDM1, OLIG3, HIC1, SOX3, FOXJ1, SOX10, KLF6, ASCL1, and PLAGL2;
(ii) PRDM1, LHX6, NEUROG3, PAX8, SOX3, KLF4, FLI1, FOXH1, FEV, SOX17, FOS, INSM1, SOX2, VVT1, SOX18, ZNF670, LHX8, OVOL1, E2F7, AFF1, HMX2, MAZ, RARA, PROP1, FOSL1, PAX5, KLF3;
(iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, !RFS, CDX4, RARA, BHLHE40, SOX3, KLF4, NR5A1, IRF4, ASCLI, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHXB, GFl1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

17. A method of increasing the conversion of a stem cell to a neuron, the method comprising:
increasing in the stem cell the level of a first neuronal-specific transcription factor selected from NGN3 and ASCL1, or a combination thereof; and
decreasing in the stem cell the level of a second neuronal-specific transcription factor selected from:
(i) ZIC2, SPl1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(ii) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296, ZNF318, ZNF570, ZNF683, ZFP36L1, HES4, ZNF777, HES5, ZIM2, ZNF579, BMP2, CRAMP1L, TOX3, FEZF2, HES3, ZNF791:
(iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, lRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1 ZNF160, ETV5, MYBL1 NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX.

18. A method of treating a subject in need thereof, the method comprising:
(a) increasing in a stem cell in the subject the level of a first neuronal-specific transcription factor selected from NEUROG3, SOX4, SOX9, KLF4, NR5A1, NEUROD1, SOX17, SMAD1, ATOH1, INSM1, NEUROG1, SOX18, RFX4, KLF7, SPS, OVOL1, NEUROG2, ERF, PRDM1, OLIG3, HIC1, SOX3, FOXJ1, SOX10, KLF6, ASCL1, and PLAGL2, or
(b) increasing in a stem cell in the subject the level of a first neuronal-specific transcription factor selected from NGN3 and ASCL1, or a combination thereof; and
increasing in a stem cell in the subject the level of a second neuronal-specific transcription factor selected from:
(i) NEUROG3, SOX4, SOX9, KLF4, NR5A1, NEUROD1, SOX17, SMAD1, ATOH1, INSM1, NEUROG1, SOX18, RFX4, KLF7, SP8, OVOL1, NEUROG2, ERF, PRDM1, OLIG3, HlC1, SOX3, FOXJ1, SOX10, KLF6, ASCL1, and PLAGL2;
(ii) PRDM1, LHX6, NEUROG3, PAX8, SOX3, KLF4, FLI1, FOXH1, FEV, SOX17, FOS, INSM1, SOX2, WT1, SOX18, ZNF670, LHX8, OVOL1, E2F7, AFF1, HMX2, MAZ, RARA, PROP1, FOSL1, PAX5, KLF3:
(iii) RUNX3, PRDM1, KLF6, PAX2, RFX3, SOX10, GATA1, KLF5, KLF1, ERF, LHX6, PHOX2B, NANOG, NR5A2, ETV3, NEUROG3, SOX4, SOX9, PAX8, lRF5, CDX4, RARA, BHLHE40, SOX3, KLF4, NR5A1, IRF4, ASCL1, GATA6, SPIB, THRB, FOXH1, NEUROD1, SOX17, CDX2, ZEB2, RARG, INSM1, FOSL1, NEUROG1, SOX1, WT1, PAX5, SOX18, POU5F1, RFX4, KLF7, NKX2-2, OVOL2, FOXJ1, PRDM14, VENTX, LHX8, GFI1, KLF17, OVOL1, OLIG3, HMX3, ZNF521, ONECUT3, OVOL3, ZNF362, AFF1, HMX2, ZNF786, GATA5, TBX3, ZNF385A, ATOH1, PROP1, SOX11, JUN, FOXE3, FERD3L, and E2F7.

19. A method of treating a subject in need thereof, the method comprising:
increasing in a stem cell in the subject the level of a first neuronal-specific transcription factor selected from NGN3 and ASCL1, or a combination thereof; and
decreasing in a stem cell in the subject the level of a second neuronal-specific transcription factor selected from:
(i) ZIC2, SPI1, GRHL2, TFAP2C, KLF8, MYB, TCF21, KLF12, TWIST1, SNAI1, RREB1, GCM2, GRHL1, ETS1, BARHL2, GRHL3, ELF3, PTF1A, GSX1, PBX2, NOTO, KLF3, ZNF311, ELMSAN1, ZNF296, PLEK, KMT2A, HES3;
(ii) HES2, SREBF1, CIC, WHSC1, VDR, HES1, ID2, TCF21, SNAI1, RREB1, GCM2, IRF3, FOXA1, GATA5, GRHL1, SOX5, DMRT1, GCM1, BARHL2, SOX13, ZEB1, PITX2, PTF1A, ZNF282, NPAS2, ZNF160, HES7, ZBED4, SALL4, GLIS3, TBX22, ZNF331, EGR4, ZIC5, ZNF710, ZNF697, ZFP36L2, ELMSAN1, ZNF296. ZNF318, ZNF570, ZNF683, ZFP36L1, HES4, ZNF777, HES5, ZIM2, ZNF579, BMP2, CRAMP1L, TOX3, FEZF2, HES3, ZNF791:
(iii) ETV1, ZIC2, GSC2, CIC, GRHL2, REST, TFAP2C, SALL1, NFKB1, ELF2, HES1, MYB, KLF12, VSX2, NFE2, SNAI1, TRERF1, RREB1, IRF1, IRF3, KLF2, MYOD1, SOX15, BARX1, GRHL1, SOX5, ETS1, SKIL, BARHL2, SOX13, ERG, GRHL3, ZNF281, ELF3, HESX1, KLF15, PITX2, PTF1A, GSX1, ZNF160, ETV5, MYBL1, NOTO, DPF1, MECOM, GLIS3, KLF3, TBX22, ESX1, ZNF337, ZFP36L2, ELMSAN1, ZNF618, ZNF296, ZNF318, ZNF570, ZNF497, ZFP36L1, HES5, BMP2, CRAMP1L, ZNF821, KMT2A, HES3, and BSX.

20. The method of any one of claims 14-19, wherein increasing the level of the first neuronal-specific transcription factor comprises at least one of:
(a) administering to the stem cell a polynucleotide encoding the first neuronal-specific transcription factor;
(b) administering to the stem cell a polypeptide comprising the first neuronal-specific transcription factor; and
(c) administering to the stern cell a fusion protein, wherein the fusion protein comprises two heterologous polypeptide domains, wherein the first polypeptide domain comprises a Cas protein, a zinc finger protein targeting the first neuronal-specific transcription factor, or a TALE protein targeting the first neuronal-specific transcription factor, and the second polypeptide domain has transcription activation activity, and wherein a gRNA targeting the first neuronal-specific transcription factor is additionally administered to the stem cell when the first polypeptide domain comprises a Cas protein.

21. The method of any one of claims 14, 16, and 18, wherein increasing the level of the second neuronal-specific transcription factor comprises at least one of:
(a) administering to the stem cell a polynucleotide encoding the second neuronal-specific transcription factor;
(b) administering to the stem cell a polypeptide comprising the second neuronal-specific transcription factor; and
(c) administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription activation activity, and wherein a gRNA targeting the second neuronal-specific transcription factor is additionally administered to the stem cell when the first polypeptide domain comprises a Cas protein.

22. The method of any one of claims 15, 17, and 19, wherein decreasing the level of the second neuronal-specific transcription factor comprises administering to the stem cell a fusion protein, wherein the fusion protein comprises two heterologous polypeptide domains, wherein the first polypeptide domain comprises a Cas protein, a zinc finger protein targeting the second neuronal-specific transcription factor, or a TALE protein targeting the second neuronal-specific transcription factor, and the second polypeptide domain has transcription repression activity, and wherein a gRNA targeting the second neuronal-specific transcription factor is additionally administered to the stem cell when the first polypeptide domain comprises a Cas protein.

23. The method of any one of claims 14-22, wherein the stem cell is directly converted to a neuron without a pluripotent stage.

24. The cell of claim 13 or the method of any one of claims 14-23, wherein the stem cell is a pluripotent stem cell, an induced pluripotent stem cell, or an embryonic stem cell.

25. A system for selecting a polynucleotide for activity as a cell type-specific transcription factor, the system comprising:
a polynucleotide encoding a reporter protein and a cell type marker;
a fusion protein, wherein the fusion protein comprises two heterologous polypeptide domains, wherein the first polypeptide domain comprises a Cas protein, and the second polypeptide domain has transcription activation activity; and
a library of guide RNAs (gRNAs), each gRNA targeting a different putative cell type-specific transcription factor.

26. The system of claim 25, wherein the cell-type specific transcription factor is a neuronal-specific transcription factor, wherein the cell type marker is a neuronal marker, and wherein the neuronal marker comprises TUBB3.

27. The system of claim 25, wherein the cell-type specific transcription factor is a muscle-specific transcription factor, wherein the cell type marker is a myogenic marker, and wherein the myogenic marker comprises PAX7.

28. The system of claim 25, wherein the cell-type specific transcription factor is a chondrocyte-specific transcription factor, wherein the cell type marker is a collagen marker, and wherein the collagen marker comprises COL2A1.

29. The system of any one of claims 25-28, wherein the reporter protein comprises mCherry.

30. An isolated polynucleotide sequence encoding the system of any one of claims 25-29.

31. A vector comprising the isolated polynucleotide sequence of claim 30.

32. A cell comprising the system of any one of claims 25-29, the isolated polynucleotide sequence of claim 30, or the vector of claim 31, or a combination thereof.

33. A method of screening for a cell type-specific transcription factor, the method comprising:
transducing a population of cells with the system of any one of claims 25-29 at a multiplicity of infection (MOD of about 0.2, such that a majority of the cells each independently includes one gRNA and targets one putative transcription factor;
determining a level of expression of the reporter protein in each cell;
determining a level of the gRNA in each cell having a high expression of the reporter protein, wherein high expression of the reporter protein is defined as being in the top 5% among the population of cells; and
selecting the putative transcription factor as a cell-type-specific transcription factor when the putative transcription factor corresponds to at least two gRNAs enriched in the cell having a high expression of the reporter protein.

34. A method of screening for a pair of cell-type-specific transcription factors, the method comprising:
transducing a population of cells with the system of any one of claims 25-29 at a multiplicity of infection (MOI) of about 0.2, such that a majority of the cells each independently includes two gRNAs and targets two putative transcription factors;
determining a level of expression of the reporter protein in each cell;
determining a level of the two gRNAs in each cell having a high expression of the reporter protein, wherein high expression of the reporter protein is defined as being in the top 5% among the population of cells; and
selecting the two putative transcription factors as a pair of cell type-specific transcription factors when the putative transcription factors correspond to at least two gRNAs enriched in the cell having a high expression of the reporter protein.

35. The method of claim 33 or 34, wherein the level of expression of the reporter protein in each cell is determined after about four days from transduction.

36. The method of any one of claims 33-35, wherein the level of expression of the reporter protein in each cell is determined by flow cytometry.

37. The method of any one of claims 33-36, wherein the level of the gRNA in each cell having a high expression of the reporter protein is determined by deep sequencing.

38. The method of any one of claims 33-37, wherein the gRNA increases the expression of the reporter protein in the cell by about 2-50% relative to a non-targeting gRNA.

39. A polynucleotide encoding a muscle-specific transcription factor selected from TWIST1, PAX3, MYOD, MYOG, SOX9, SOX10, and DMRT1.

40. A system for increasing expression of a muscle-specific gene, the system comprising:
(a) a muscle-specific transcription factor selected from TWIST1, PAX3, MYOD, MYOG, SOX9, SOX10, and DMRT1; or
(b) a fusion protein, wherein the fusion protein comprises two heterologous polypeptide domains, wherein the first polypeptide domain comprises a Cas protein, a zinc finger protein targeting a muscle-specific transcription factor selected from TWIST1, PAX3, MYOD, MYOG, SOX9, SOX10, and DMRT1, or a TALE protein targeting a muscle-specific transcription factor selected from TWIST1, PAX3, MYOD, MYOG, SOX9, SOX10, and DMRT1, wherein the second polypeptide domain has an activity selected from transcription activation activity, transcription release factor activity, histone modification activity, nucleic acid association activity, methylase activity, and demethylase activity, and wherein the system further includes a gRNA targeting a muscle-specific transcription factor selected from TWIST1, PAX3, MYOD, MYOG, SOX9, SOX10, and DMRT1 when the first polypeptide domain comprises a Cas protein.

41. The system of claim 40, wherein the fusion protein comprises ^{VP64}dCas9^{VP54} or dCas9-p300.

42. An isolated polynucleotide encoding the system of any one of claims 40-41.

43. A vector comprising the isolated polynucleotide of claim 42.

44. A cell comprising the isolated polynucleotide of claim 42 or the vector of claim 43.

45. A method of increasing differentiation of a stem cell into a myoblast, the method comprising:
increasing in the stem cell the level of a muscle-specific transcription factor selected from TWIST1, PAX3, MYOD, MYOG, SOX9, SOX10, and DMRT1.

46. A method of treating a subject in need thereof, the method comprising:
increasing in a stem cell from the subject the level of a muscle-specific transcription factor selected from TWISTI, PAX3, MYOD, MYOG, SOX9, SOX10, and DMRT1.

47. The method of claim 45 or 46, wherein increasing the level of the muscle-specific transcription factor comprises at least one of;
(a) administering to the stem cell a polynucleotide encoding the muscle-specific transcription factor;
(b) administering to he stem cell a polypeptide comprising the muscle-specific transcription factor; and
(c) administering to the stem cell a fusion protein, wherein the fusion protein comprises two heterologous polypeptide domains, wherein the first polypeptide domain comprises a Cas protein, a zinc finger protein targeting the muscle-specific transcription factor, or a TALE protein targeting the muscle-specific transcription factor, wherein the second polypeptide domain has transcription activation activity, and wherein a gRNA targeting the muscle-specific transcription factor is additionally administered when the first polypeptide domain comprises a Cas protein.
